(19) United States
(12) Patent Application Publication
(10) Pub. No.: US 2025/0250309 A1
(43) Pub. Date: Aug. 7, 2025

(54) CHIMERIC EFFEROCYTIC RECEPTORS IMPROVE APOPTOTIC CELL CLEARANCE AND ALLEVIATE INFLAMMATION

(71) Applicant: University of Virginia Patent Foundation, Charlottesville, VA (US)

(72) Inventors: Kodimangalam S. Ravichandran, St. Louis, MO (US); Sho Morioka, Charlottesville, VA (US)

(21) Appl. No.: 18/856,812

(22) PCT Filed: Apr. 20, 2023
(86) PCT No.: PCT/US2023/066021

§ 371 Date: Oct. 14, 2024

Related U.S. Application Data
(60) Provisional application No. 63/363,295, filed on Apr. 20, 2022.

(51) Int. Cl. C07K 14/47; A01K 67/0278; A61K 38/00; A61P 13/12; C07K 14/705; C12N 5/0786

(52) U.S. Cl. CPC C07K 14/47; A01K 67/0278; A61P 13/12; C07K 14/705; C12N 5/0645; A01K 2217/052; A01K 2227/105; A01K 2227/40; A61K 38/00; C07K 2319/00; C12N 2510/00

(57) ABSTRACT

The present invention provides compositions and methods to increase efferocytosis and treat disease, including treating inflammation.

### PRIORITY APPLICATION

This application claims the benefit of priority to U.S. Provisional Patent Application Ser. No. 63/363,295, filed Apr. 20, 2022, the content of which is incorporated herein by reference in its entirety.

### GOVERNMENT GRANT SUPPORT

This invention was made with government support under GM122542 awarded by the National Institutes of Health. The government has certain rights in the invention.

### INCORPORATION BY REFERENCE OF SEQUENCE LISTING

A Sequence Listing is provided herewith as an xml file, “2327851.xml” created on Apr. 20, 2023 and having a size of 51,324 bytes. The content of the xml file is incorporated by reference herein in its entirety.

### BACKGROUND OF THE INVENTION

We turnover billions of cells daily through apoptotic cell death that are cleared by phagocytes via ‘efferocytosis’. Defects in efferocytosis are now linked to many inflammatory diseases.

### SUMMARY

In the healthy state, our bodies turnover billions of cells daily through apoptotic cell death that is coupled to clearance of these dying cells by phagocytes via the process of ‘efferocytosis’. Defects in efferocytosis have been linked to many inflammatory diseases. Herein is provide a new strategy to boost efferocytosis in vitro and in vivo, denoted ‘chimeric receptor for efferocytosis’ (CHEF). The engineering of CHEF involves fusing a particular signaling domain within the cytoplasmic adapter ELMO1 to the extracellular phosphatidylserine recognition domains of the efferocytic receptors BAI1 or TIM4, generating BELMO and TELMO, respectively. BELMO and TELMO expressing phagocytes displayed a striking increase in efferocytosis of apoptotic cells. Transgenic mice and transgenic zebrafish with tissue or cell type specific expression of BELMO, targeting different professional and non-professional phagocytes, potently boosted efferocytosis in vivo. In three different mouse models of inflammation, BELMO expression attenuated colitis in the gut, hepatotoxicity in the liver, and nephrotoxicity in the kidney. Mechanistic studies on the beneficial effects of BELMO expression identified a need for improving protein-folding/protein-overload-associated toxicity during efferocytosis. Specifically, BELMO increased ER resident enzymes and chaperones to overcome proteotoxicity, which was validated in ER-stress induced renal ischemia-reperfusion injury in vivo. Finally, introduction of TELMO after onset of kidney injury significantly reduced fibrosis. Collectively, these data advance a concept of chimeric efferocytic receptors to boost efferocytosis and dampen inflammation

Provided herein are composition, such as those comprising chimeric receptors for efferocytosis (CHEF) that enhance apoptotic cell clearance, and methods of their use, including methods to boost efferocytosis via CHEF which attenuates multiple inflammatory insults in vivo, and improving the outcome in ongoing disease in using CHEF/CHEF expression.

One embodiment provides a fusion protein comprising an intracellular domain of engulfment protein ELMO1 and an extracellular phosphatidylserine recognition domain of an efferocytic receptor. In one embodiment, the intracellular domain of the engulfment protein ELMO1 is human or 90% identity thereto. In one embodiment, the efferocytic receptor is BAI1 or TIM4. In one embodiment, the BAI1 extracellular phosphatidylserine recognition domain is human or 90% identity thereto. In one embodiment, the TIM4 extracellular phosphatidylserine recognition domain is human or 90% identity thereto. In one embodiment, amino terminal portion of the amino acid sequence of BAI1 (e.g., 1-1180 of BAI1) is directly fused to amino acid sequence of ELMO1 (e.g., 533-727 of ELMO1). In another embodiment, amino terminal portion of the amino acid sequence of TIM4 (e.g., 1-301 of TIM4) is directly fused to amino acid sequence ELMO1 (e.g., 533-727 of ELMO1). One embodiment provides an RNA or DNA sequence coding for the fusion protein(s) described herein.

One embodiment provides for a transgenic cell or transgenic non-human animal comprising a transgene which codes for and expresses a fusion protein described herein. In one embodiment, the transgene is stably or transiently expressed. In one embodiment, the cell is a phagocyte. In one embodiment, the phagocyte is a professional phagocyte. In one embodiment, the professional phagocyte is a macrophage, neutrophil, monocytes, dendritic cell or osteoclast. In one embodiment, the phagocyte is a non-professional phagocyte. In one embodiment, the non-professional phagocyte is an epithelial cell, fibroblast, glial cell or endothelial cell. In one embodiment, the animal is a zebrafish or a mouse.

One embodiment provides a method to increase efferocytosis/phagocytosis of apoptotic cells in vitro and/or in vivo comprising contacting a cell or administering to a subject in need thereof any one of the fusion proteins disclosed herein or an RNA or DNA coding for the fusion protein.

Another embodiment provides a method to decrease inflammation in a subject in need thereof comprising administering any one of the fusion proteins disclosed herein or an RNA or DNA coding for the fusion protein. In one embodiment, the inflammation is caused by colitis in the gut, hepatotoxicity in the liver or nephrotoxicity in the kidney.

One embodiment provides a method to treat colitis in the gut, hepatotoxicity in the liver, and/or nephrotoxicity in the kidney in a subject in need thereof comprising administering any one of the fusion proteins disclosed herein or an RNA or DNA coding for the fusion protein.

Another embodiment provides a method to reduce fibrosis during kidney injury comprising administering any one of the fusion proteins disclosed herein or an RNA or DNA coding for the fusion protein.

One embodiment provides a method to increase expression of an ER-resident enzyme and/or chaperone in a cell comprising contacting said cell or administering to a subject in need thereof any one of the fusion proteins disclosed herein or an RNA or DNA coding for the fusion proteins. In one embodiment, the cell is a macrophage. In one embodiment, the ER-resident enzyme and/or chaperone is one or more of Atp2a3, Hsp70-type chaperone BiP, DNAJC3 or Vimp.

In one embodiment, the contacting or administering results in increase of protein quality/improved folding of protein and/or increased degradation of misfolded protein. In embodiment, the contacting or administering results in a decrease in proteotoxicity.

In one embodiment, the subject has a pathological condition. In one embodiment, the pathological condition is selected from the group consisting of including tissue injury, infection, neurodegenerative diseases, and autoimmune diseases. In one embodiment, the pathological condition is atherosclerosis, systemic lupus erythematosus and atherosclerosis or ulcerative colitis.

One embodiment provides a method to treat kidney disease comprising administering to a subject in need thereof any one of the fusion proteins disclosed herein or a RNA or DNA coding for the fusion protein. In one embodiment, after administration plasma creatinine, blood urea nitrogen (BUN) or combination thereof is decreased compared to prior to administration.

In one embodiment, administration is local, such as directly to a tissue or organ. In another embodiment, the administration is systemic. In one embodiment, RNA is administered. In another embodiment, DNA is administered. In one embodiment the DNA is in an expression vector, including a viral vector.

In one embodiment, a cell expressing a fusion protein described herein is administered to a subject in thereof. For example, cells can be removed from a patient, those cells can be then transfected, such as viral mediated transfection, with an RNA or DNA molecule that codes for the fusion protein. Monocytes and/or macrophages expressing the fusion protein can be isolated from the transfected cells and then those isolated cells can be administered to the subject. In one embodiment, the cell is autologous. In another embodiment, the cell is allogeneic.

In one embodiment, the fusion protein is coded for on one or more nucleic acid sequences.

### BRIEF DESCRIPTION OF THE DRAWINGS

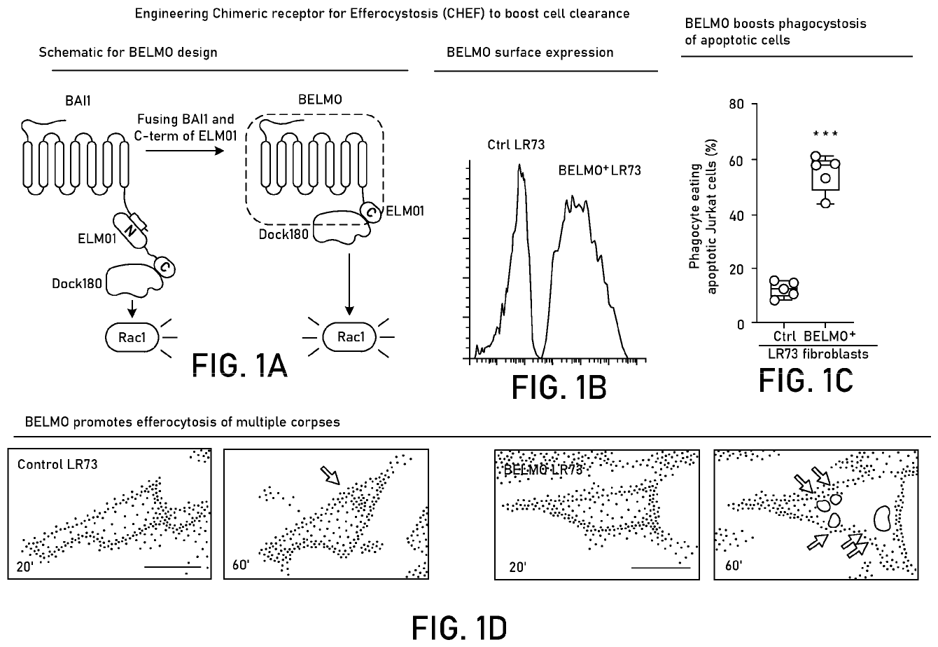

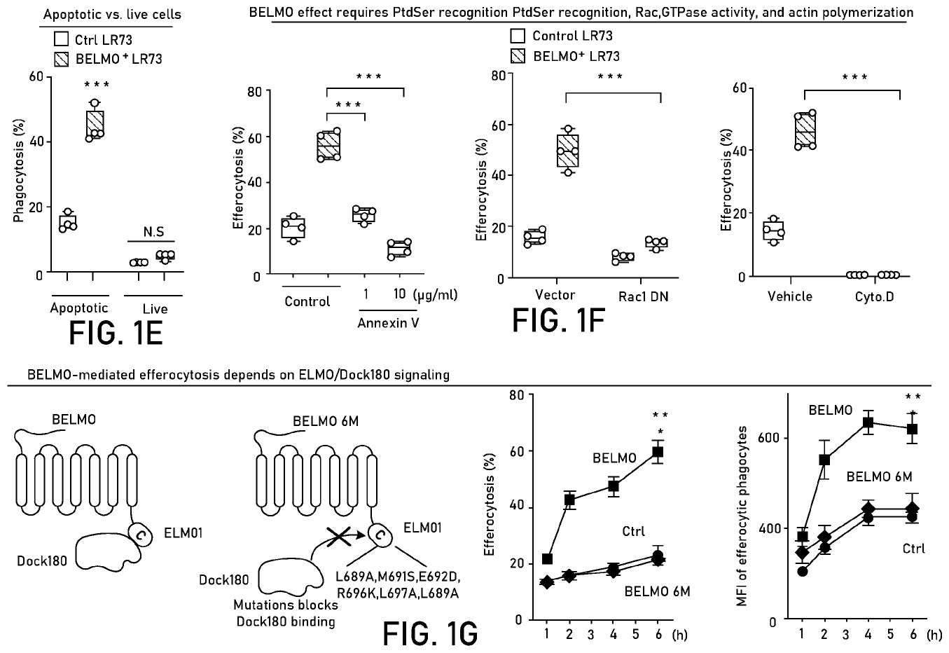

FIGS. 1A-1G. Chimeric efferocytosis receptor boosts phagocytosis.
(A) Schematic for the design of BELMO. The cytoplasmic tail of BAI1 was truncated to delete the native ELMO binding helix region and was directly fused to the C-term domain of ELMO, which is sufficient to interact with Dock180 and activate Rac1 and intracellular signaling.
(B) Cell surface expression of BELMO as confirmed using BAI1 antibody.
(C) BELMO boosts apoptotic cell uptake. LR73 cells were co-cultured with CypHer5E-labeled apoptotic Jurkat cells for 2 h and phagocytosis was measured by flow cytometry. ***p<0.001. Data are representative of five independent experiments with three replicates per condition.
(D) Efferocytosis of multiple corpses by BELMO⁺ cells. LR73 cells were co-cultured with CypHer5E-labeled apoptotic thymocytes followed by time-lapse imaging. Scale Bar=20 μm.
(E) Specificity of BELMO for apoptotic cells. Control or BELMO⁺ LR73 cells were mixed with CypHer5E-labeled live or apoptotic Jurkat cells for 2 h and their efferocytosis analyzed. ***p<0.001. N.S., Not significant. Representative of 4 independent experiments (in triplicates).
(F) BELMO requires phosphatidylserine on apoptotic cells for efferocytosis. Apoptotic Jurkat cells were incubated with Annexin V for 30 min after CypHer5E staining, mixed with Control or BELMO⁺ LR73 cells, and their phagocytosis was assessed after 2 h. ***p<0.001. Data are representative of four independent experiments with three replicates per condition.
(G) Control or BELMO⁺ LR73 cells expressing a dominant-negative form of Rac1 (N17) were tested for efferocytosis with CypHer5E-labeled apoptotic Jurkat cells for 2 h. ***p<0.001. Data are representative of four independent experiments with three replicates per condition.
   Blocking actin polymerization impairs BELMO mediated efferocytosis. Control or BELMO⁺ LR73 cells were treated with 1 M of Cytochalasin D for 1 h, then cocultured with CypHer5E-labeled apoptotic Jurkat cells for 2 h. Phagocytosis was measured by flow cytometry. ***p<0.001. Data are representative of four independent experiments with three replicates per condition.
   BELMO requires DOCK180 binding for efferocytosis. Schematic of BELMO 6M mutation within a region of ELMO that disrupts Dock180 binding. BELMO LR73 cells or 6M cells were co-cultured with CypHer5E-labeled apoptotic Jurkat cells for the indicated times. Data are shown as % engulfment and mean fluorescence intensity (MFI), an indirect measure of corpse burden indicated by apoptotic cell-derived fluorescence per phagocyte. ***p<0.001. Data are representative of three independent experiments with three replicates per condition.

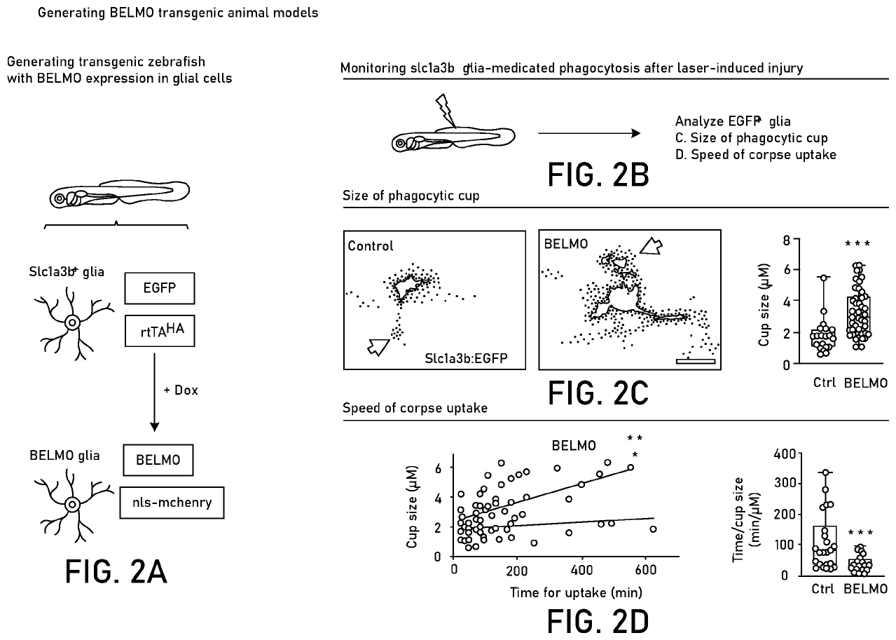

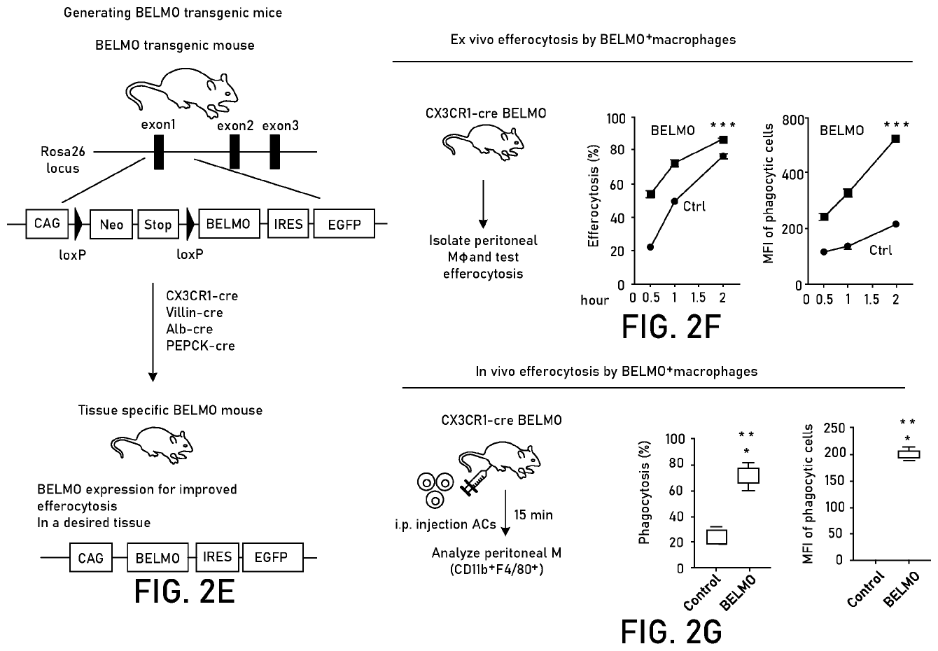

FIGS. 2A-2G. Tissue-specific BELMO expression promotes efferocytosis in vivo
(A, B) Schematic for BELMO expression in zebrafish and laser-induced injury. Illustration of DNA constructs that were injected into one-cell stage zebrafish embryos. At 3 days post-fertilization, slc1a3b-eGFP reporter zebrafish larvae were treated with 10 μM doxycycline to induce BELMO and mCherry in slc1a3b-positive glia for 24 hours. Injury was created using a 435-nm pulsed nitrogen dye laser and the glia were imaged every 12 minutes for 10 hours. BELMO⁺ glia were identified by the expression of nuclear mCherry.
(C) BELMO⁺ glia have larger phagocytic cups. Glia were identified by eGFP expression (along with their morphology). Cells were pseudo-colored to better visualize fine processes. Arrows indicate phagocytic cup. Quantification of the size of phagocytic cups is shown on the right. ***p<0.001. BELMO-negative n=8 cells with n=22 phagocytic cups from n=8 larvae, and for BELMO-positive n=12 cells with n=45 phagocytic cups from n=8 larvae. Scale bar=10 μm.
(D) Speed of corpse uptake. Via live imaging, the duration of the phagocytic cups to be taken up into the cells was quantified from initiation of phagosome formation to corpse internalization. Duration and size of the phagocytic cup (left) and duration normalized for size of the phagocytic cup (right). ***p<0.001. BELMO-negative n=8 cells with n=22 phagocytic cups from n=8 larvae, and for BELMO-positive n=12 cells with n=45 phagocytic cups from n=8 larvae.
(E) Schematic for the generation of BELMO^{Tg} mice.
(F-G) BELMO expression boosts efferocytosis by macrophages. BELMO^{Tg} mice were crossed with Cx3cr1-cre mice. For ex vivo efferocytosis, peritoneal macrophages were isolated and incubated with CypHer5E-labeled apoptotic Jurkat cells for the indicated times, and efferocytosis assessed by flow cytometry (F). CypHer5E-labeled apoptotic Jurkat cells were injected intraperitoneally, and 15 min later, and the engulfment by CD11b⁺ F4/80hi macrophages was assessed (G). ***p<0.001. Data are representative of four independent experiments with 2 replicates per condition.

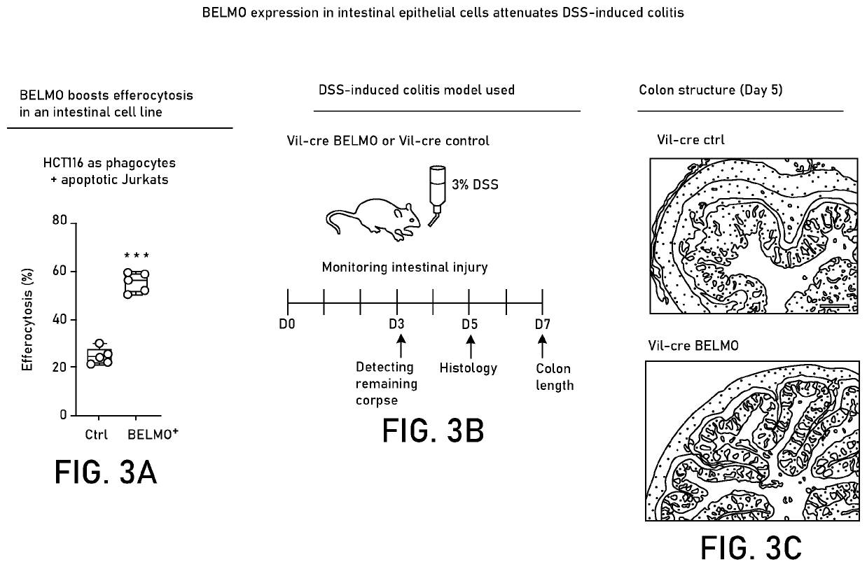

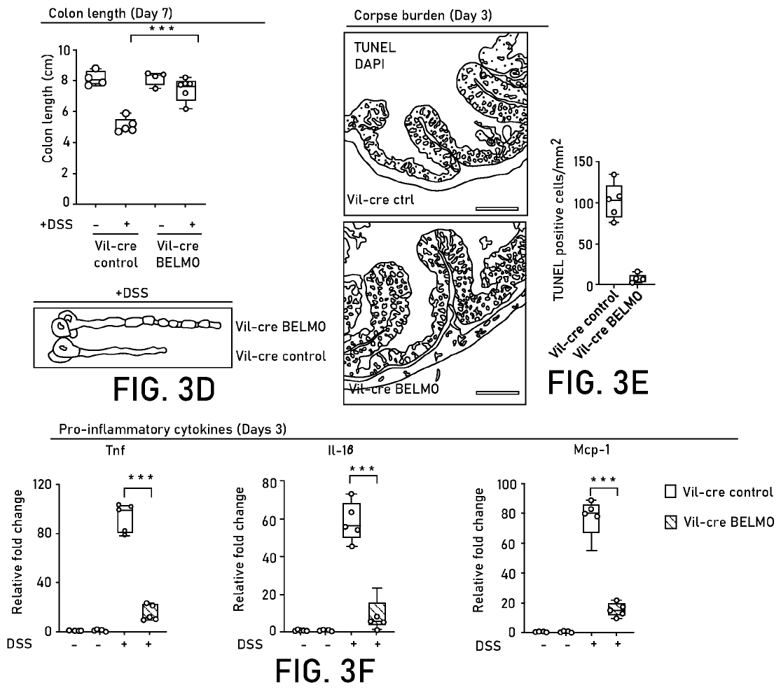

FIGS. 3A-3F. BELMO attenuates DSS-induced colitis.
(A) BELMO boosts apoptotic cell uptake in a colonic epithelial cell line. BELMO expressing HCT116 cells were co-cultured with CypHer5E-labeled apoptotic Jurkat cells for 2 h. Phagocytosis was measured by flow cytometry. ***p<0.001. Data are representative of five independent experiments with three replicates per condition.
(B-F) BELMO expression in intestinal epithelial cells (IEC) dampens DSS-induced colitis. BELMO^{Tg} mice were crossed with Villin-cre mice for IEC expression of BELMO. Mice were treated with 3% DSS and monitored for 7 days and analyzed for the indicated parameters (B). On day 5, colon samples were collected, and analyzed by H&E staining (C). On day 7, the length of the colon was better retained in BELMO expression conditions (D). On day 3, colon samples were collected and cell death was analyzed by TUNEL staining. 10 sections at 200 μm intervals per colon were counted for TUNEL positive cells. Data are representative of 5 mice in each group (E). mRNA was isolated from the colon and the expression of proinflammatory cytokines was analyzed by qPCR. Data are representative of 5 mice in each group (F). Data are representative of 5 mice in each group. ***p<0.001. Scale bars 200 μm.

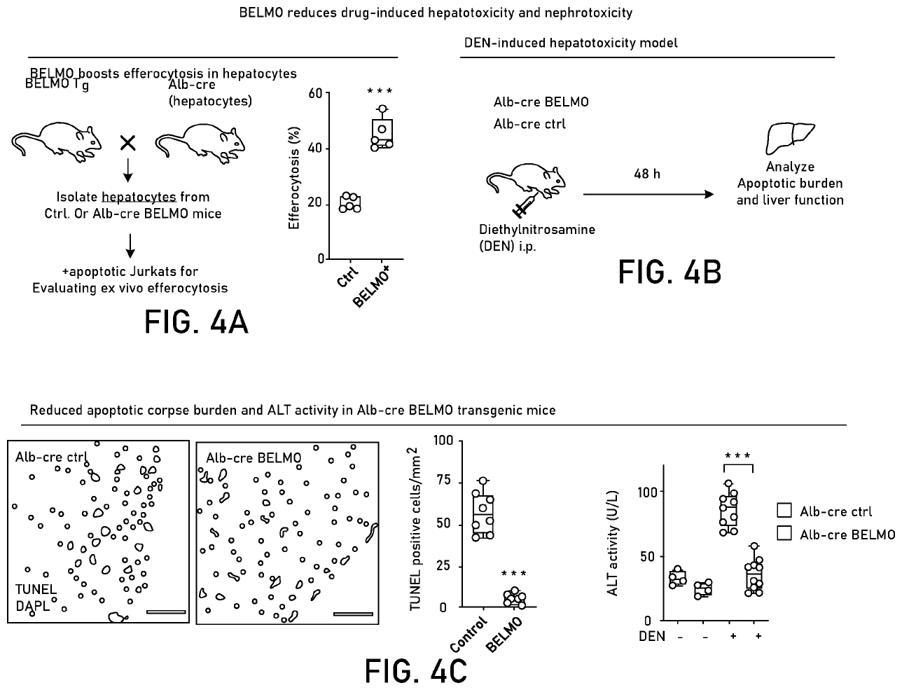

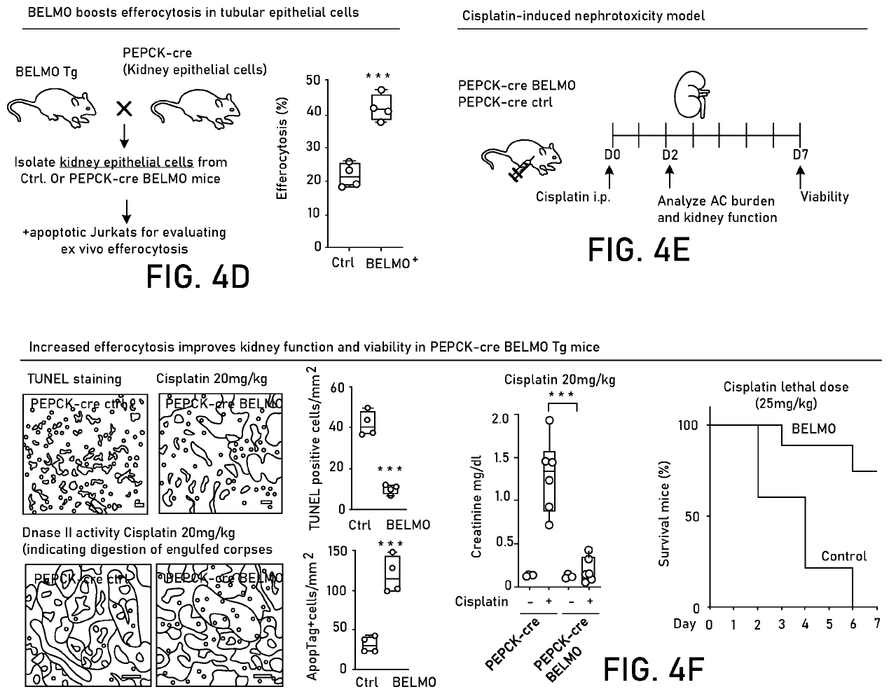

FIGS. 4A-4F. BELMO ameliorates drug-induced hepatotoxicity and nephrotoxicity.
(A) BELMO boosts apoptotic cell uptake in primary hepatocytes. BELMO^{Tg} mice were crossed with Alb-cre mice to induce BELMO expression in hepatocytes. Primary hepatocytes were isolated and incubated with CypHer5E-labeled apoptotic Jurkat cells for 2 h. Phagocytosis was measured by flow cytometry. ***p<0.001. Data are representative of five independent experiments with three replicates per condition.
(B, C) BELMO dampens diethylnitrosamine (DEN)-induced hepatotoxicity. Mice were treated with intraperitoneal injection of 100 mg/kg of DEN (B). 48 h later, liver samples were collected and analyzed by TUNEL staining. Liver damage was analyzed by ALT activity assay (C). ***p<0.001. Data are representative of three independent experiments with 4-10 mice in each group per experiment. ***p<0.001
(D) BELMO boosts apoptotic cell uptake in primary tubular epithelial cells (TEC) of the kidney. BELMO^{Tg} mice were crossed with PEPCK-cre mice for inducing BELMO expression in TEC. Primary TECs were isolated and incubated with CypHer5E-labeled apoptotic Jurkat cells for 2 h and phagocytosis assessed. Data are shown as % engulfment. ***p<0.001. Data are representative of four independent experiments with two replicates per condition.
(E, F) BELMO alleviates cisplatin-induced nephrotoxicity. Schematic of the nephrotoxicity model (E). 48 h after cisplatin treatment (20 mg/kg body weight), kidney samples were collected and analyzed by TUNEL staining (cell death) and DNase II-mediated DNA cleavage in phagolysosomes (efferocytosis). Plasma samples were collected and assessed for creatinine in circulation. After cisplatin treatment (25 mg/kg body weight), the viability over 7 days was monitored (F). Data are representative of three independent experiments with 3-10 mice in each group. ***p<0.001, **p<0.005.

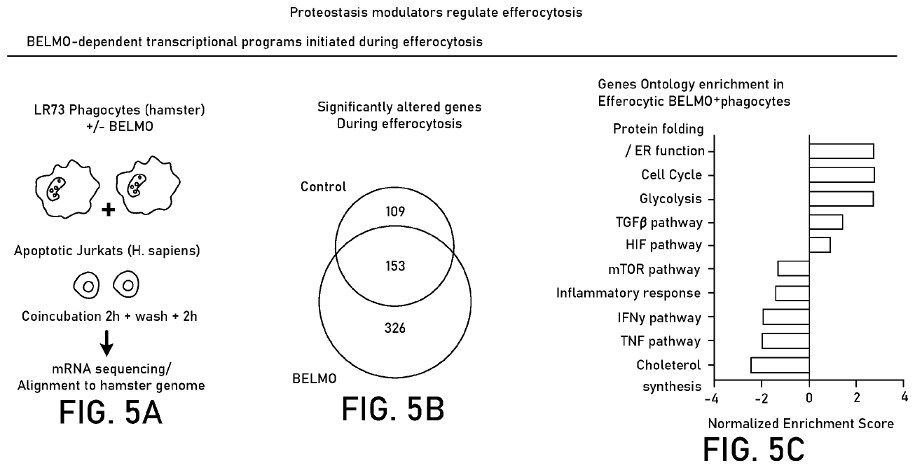

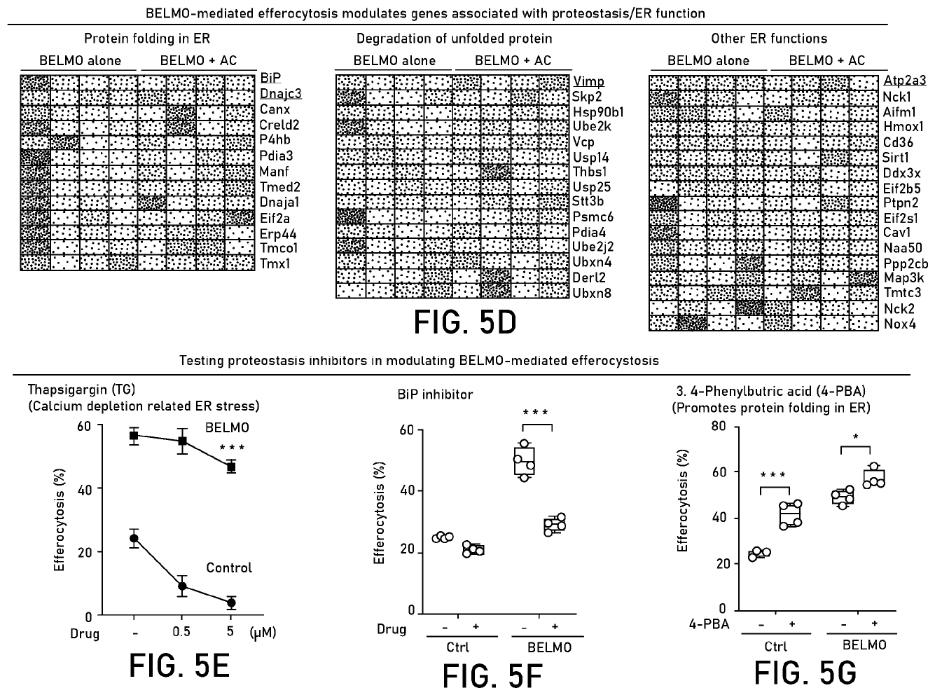

FIGS. 5A-5G. Protein folding modulators regulate efferocytosis.
(A) Transcriptomics analysis of BELMO expressing phagocytes during efferocytosis. Control or BELMO⁺ LR73 cells were incubated with apoptotic human Jurkat cells for 2 h, the unbound/free apoptotic cells were removed by washing, and LR73 cells were cultured for an additional 2 h. The mRNA from LR73 cells was then isolated and RNAseq was performed.
(B) Venn diagram illustrating the shared and distinct genes induced during efferocytosis between control and BELMO⁺ LR73 cells. Adjustedp value <0.05, and Log 2Fold change >0.3 or <−0.3.
(C) Gene ontology analysis of upregulated (red) and downregulated (blue) genes in BELMO⁺ phagocytes. The bidirectional plots represent the normalized enrichment score. Protein folding and ER function gene sets were manually curated from public gene clusters (GO:0034976).
(D) BELMO⁺ phagocytes upregulate genes involved in protein folding and ER function after efferocytosis. Adjustedp <0.05.
(E-G) Control and BELMO⁺ LR73 cells were pretreated with thapsigargin (E), the BiP inhibitor epigallocatechin gallate (F), or 4-PBA (G). Cells were co-cultured with CypHer5E-labeled apoptotic Jurkat cells for 2 h. Phagocytosis was measured by flow cytometry. ***p<0.001.*p<0.05. Data represent at least four independent experiments with two replicates per condition.

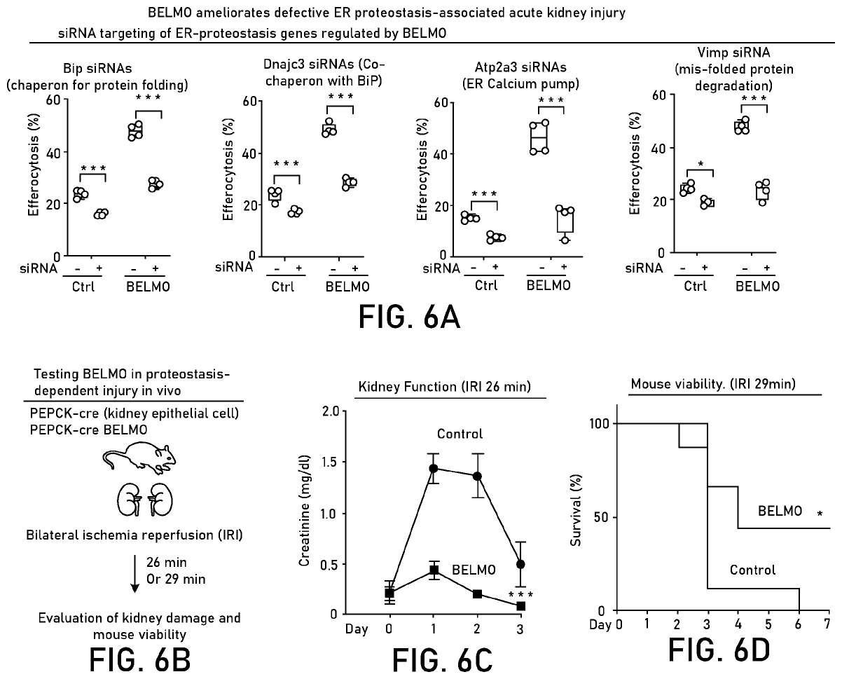

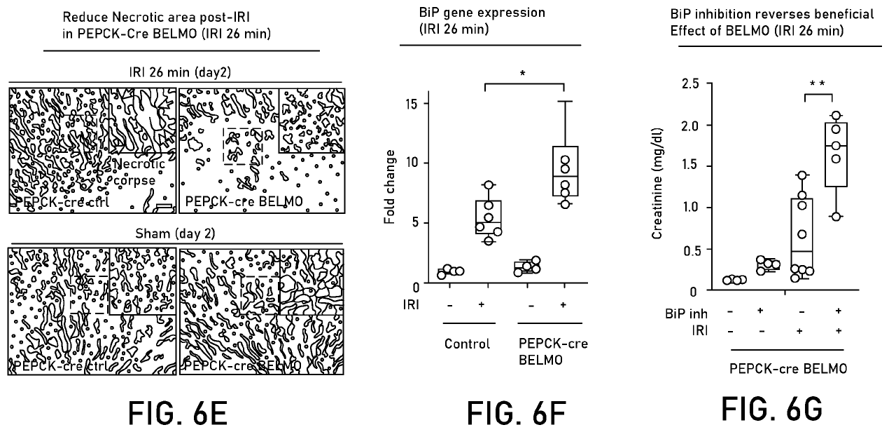

FIGS. 6A-6G. BELMO ameliorates defective ER proteostasis-associated acute kidney injury.
(A) siRNA-targeting of BiP, Dnajc3, Atp2a3 and Vimp suppress efferocytosis. LR73s (scramble or gene targeting siRNAs) were co-cultured with CypHer5E-labeled apoptotic Jurkat cells for 2 h and efferocytosis measured. ***p<0.001. ***p<0.05. Data are representative of four independent experiments with 2-3 replicates per condition.
(B) BELMO reduces acute kidney injury after ischemia-reperfusion injury (IRI). BELMO^{Tg} mice were crossed with PEPCK-cre mice for targeting expression of BELMO to kidney tubular epithelial cells. Bilateral IRI injury was induced by clamping the renal pedicles for 26 m or 29 min. The clamps were then removed, and the wound sutured after restoration of blood flow (as visually observed). Kidneys were allowed to reperfuse for the indicated times.
(C) After IRI, blood samples were collected and plasma creatinine was analyzed over three days. Data are representative of at least 10 mice per genotype.
(D) BELMO improves mouse viability after IRI-induced acute kidney injury. After IRI (29 min), mice were monitored over 7 days for mortality. Data are representative of three independent experiments with 8 control mice and 10 BELMO transgenic mice.
(E) Kidney samples were collected and analyzed by H&E staining. Data are representative of at least 10 mice per condition. Scale bar 200 μm.
(F) BELMO increases BiP expression during acute kidney injury. 24 h after IRI surgery, kidneys were collected and mRNA extracted. Expression level of BiP was analyzed via qPCR. *p<0.05. Data are representative of at least two independent experiments with 3-6 mice per condition.
(G) BiP inhibition dampens the beneficial effect of BELMO after bilateral IRI. BiP inhibitor EGCG (50 mg/kg) was intraperitoneally administered 1 day before IRI and just after the surgery. **p<0.01. Data are representative of three independent experiments with 8 control mice and 5 BELMO transgenic mice.

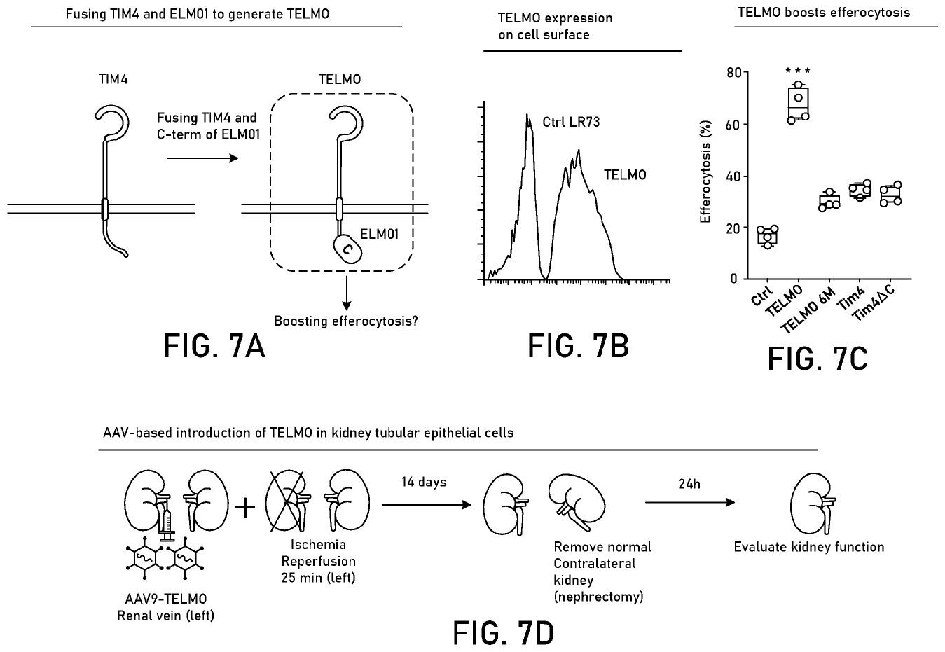

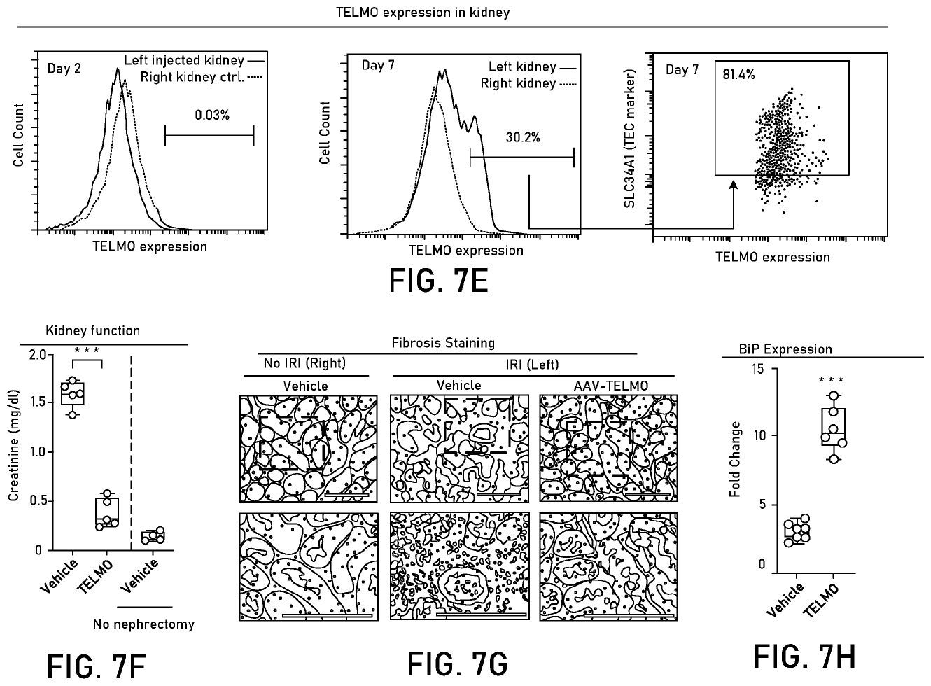

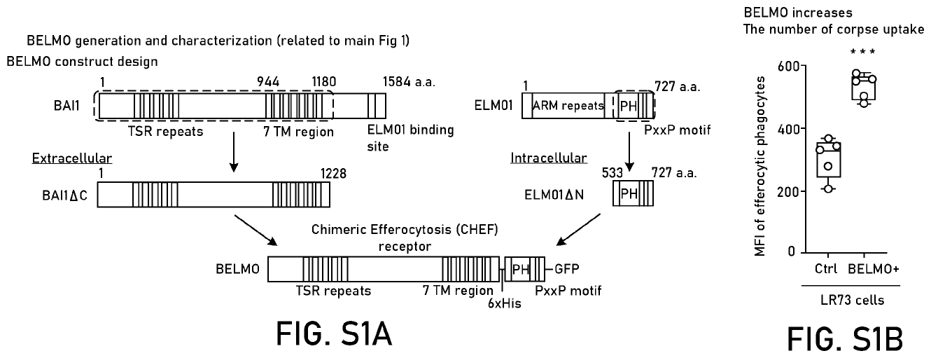

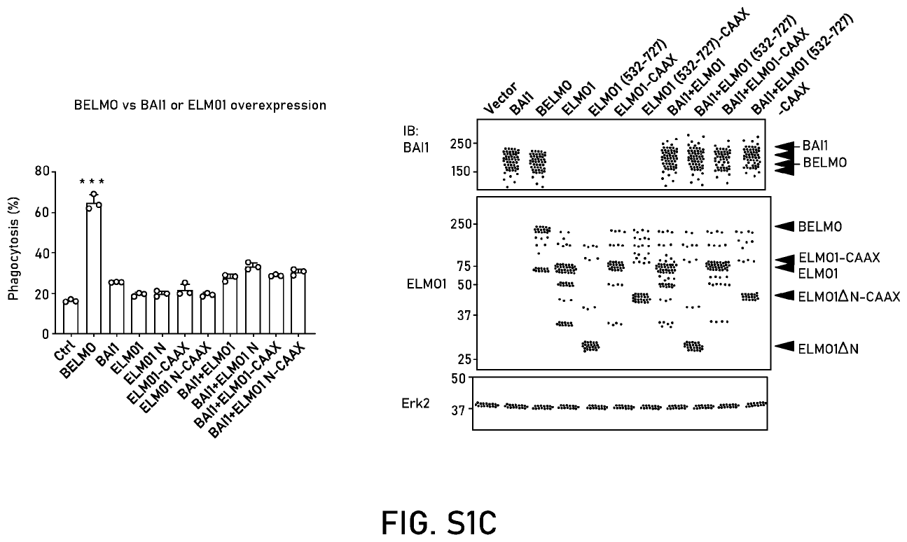

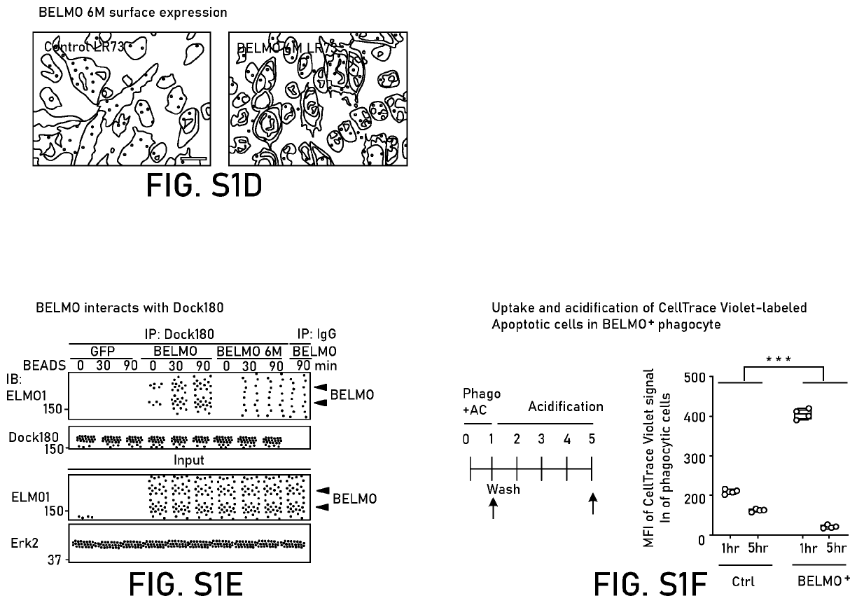

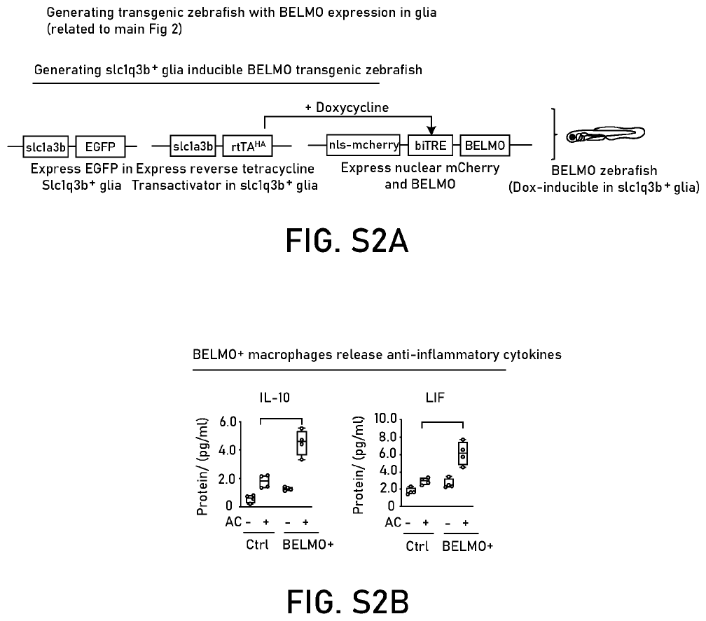

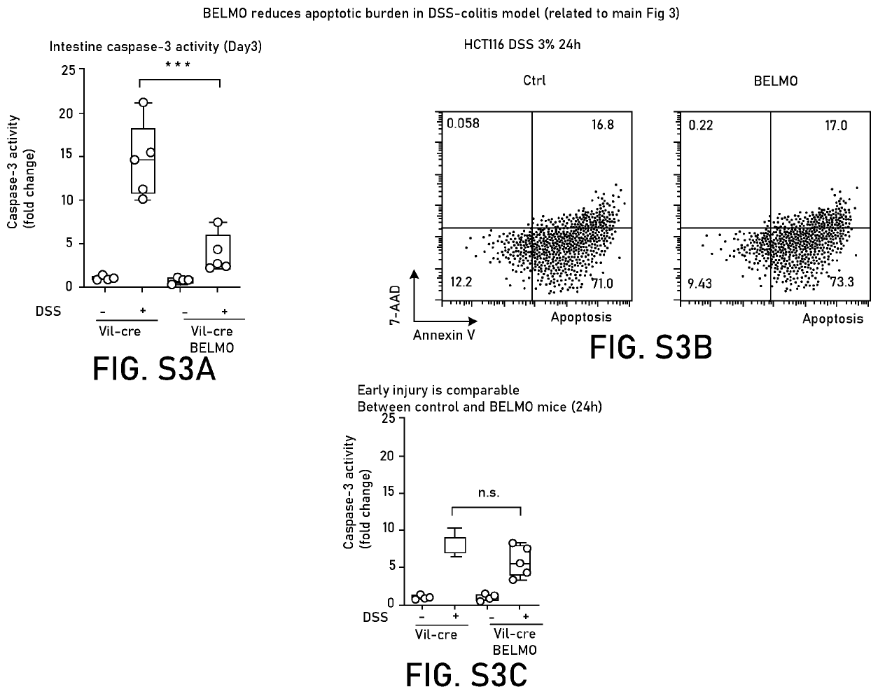

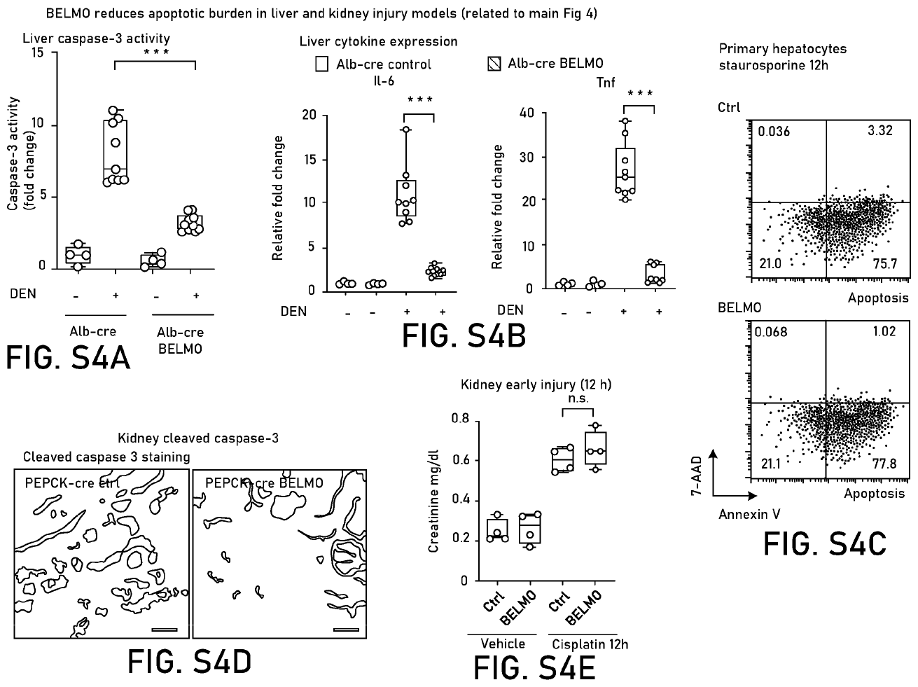

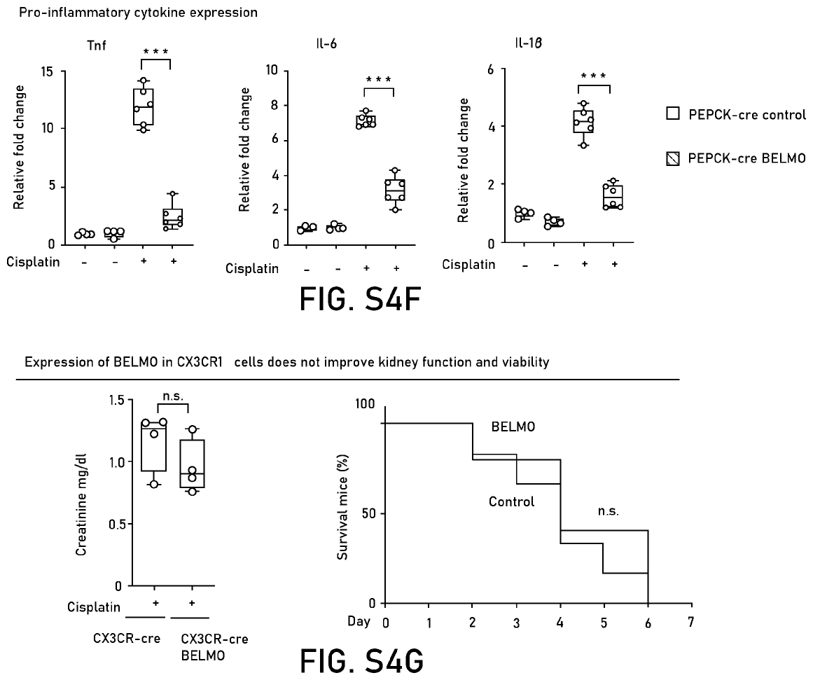

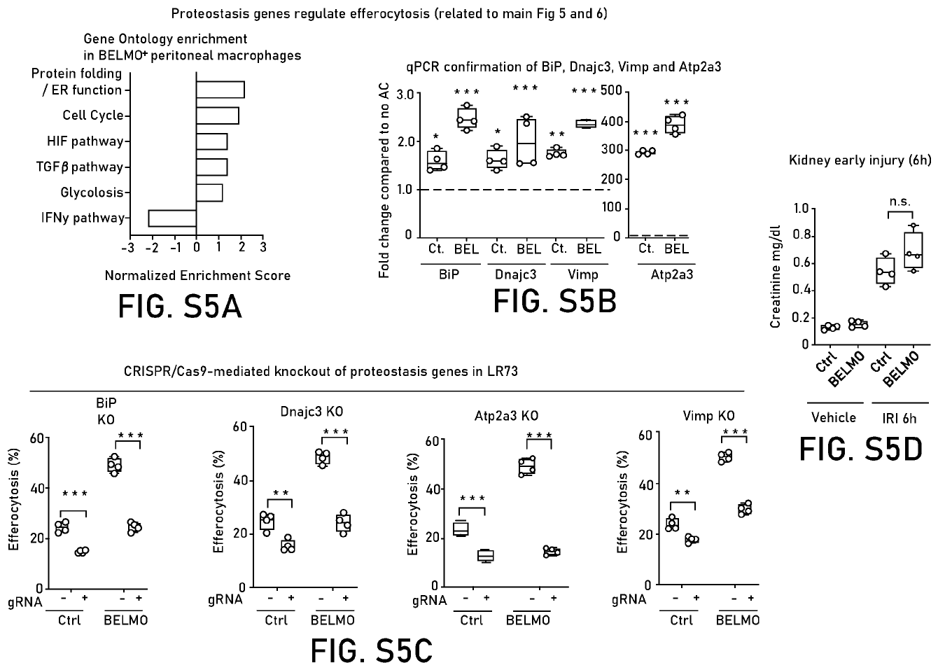

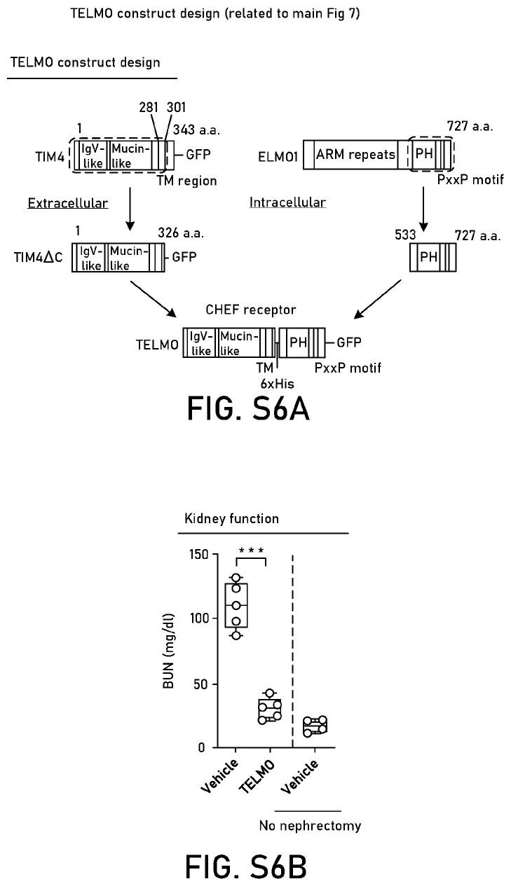

FIGS. 7A-7H. AAV-transduced TELMO ameliorates kidney disease progression.
(A) Schematic of TELMO generation.
(B) Cell surface expression of TELMO was confirmed by TIM4 antibody and flowcytometry.
(C) TELMO boosts efferocytosis. TELMO, 6M, TIM4 and TIM4 lacking intracellular domain LR73 cells were co-cultured with CypHer5E-labeled apoptotic Jurkat cells for 2 h. Phagocytosis was measured by flow cytometry. ***p<0.001. Data are representative of four independent experiments with three replicates per condition.
(D-H) Adeno-associated virus (AAV)-mediated delivery via adenovirus ameliorates chronic kidney disease induced by IRI. Schematic of the IRI model used (D). Left kidney was clamped and simultaneously AAV9-TELMO-GFP or control virus was introduced via renal vein injection; 25 min later, the clamp was removed. 14 days later, the contralateral right kidney was removed, and the left kidney function was evaluated after 24 h. After AAV injection, TELMO expression was monitored on day 2 and day 7 by detecting GFP⁺ cells via flow cytometry. On day 7, GFP⁺ cells were analyzed within SLC34A1⁺ tubular epithelial cell population. Data are representative of at least four independent experiments
(E). Blood samples were collected for plasma creatinine quantification. Data are representative of at least three independent experiments with 5 mice per condition (F). Kidney fibrosis was visualized by Masson Trichrome staining (G). Kidney samples were collected on day 7 and the expression level of Bip was analyzed via qPCR. Data are representative of 6 mice per condition. (H). ***p<0.001. Scale bar 200 μm.

FIG. S1 BELMO boosts efferocytosis (related to FIG. 1)
(A) Schematic of the BELMO construction.
(B) LR73 cells were co-cultured with TAMRA-labeled apoptotic Jurkat cells for 2 h, washed and the MFI of TAMRA⁺ phagocytes assessed. ***p<0.001. Data are representative of three independent experiments with three replicates per condition.
(C) LR73 cells expressing various phagocytosis related genes were co-cultured with CypHer5E-labeled apoptotic Jurkat cells for 2 h, and phagocytosis measure by flow cytometry. Data are shown as % engulfment. ***p<0.001. Data are representative of three independent experiments with three replicates per condition. Expression levels of the genes were shown by immunoblotting.
(D) Cell surface expression of BELMO 6M-GFP. Scale bar 20 μm.
(E) LR73 cells were stimulated with carboxylate-modified beads for the indicated times. Cell lysates were prepared and Dock180 interaction was addressed by immunoprecipitation.
(F) LR73 cells were co-cultured with CellTraceViolet-labeled apoptotic Jurkat cells for 1 h. Cells were washed and left for 4 h to allow digestion of corpse. MFI of CellTraceViolet⁺ phagocytes are shown. ***p<0.001. Data are representative of three independent experiments with three replicates per condition.

FIG. S2 Generation and validation of CHEF animal models, related to FIG. 2
(A) BELMO enhances uptake of cellular debris after laser-induced injury in zebrafish. Illustration of DNA constructs that were injected into one-cell stage zebrafish embryos.
(B) BELMO expressing peritoneal macrophages were cultured with apoptotic cells for 2 h. Apoptotic cells were then removed by 3×PBS wash, 1 ml of fresh media was added, and phagocytes were cultured for an additional 12 h. Supernatants were then collected for Luminex analysis. **p<0.01. *p<0.05.

FIG. S3 BELMO ameliorates DSS-induced colitis, related to FIG. 3
(A) BELMO expression in intestinal epithelial cells (IEC) dampens DSS-induced colitis. BELMO^{Tg} mice were crossed with Villin-cre mice for IEC expression of BELMO. Mice were treated with 3% DSS. On day 3, colonic epithelial cell samples were collected, and tissue lysates were prepared. Caspase-3 activity was determined by Caspase Glo 3/7 kit. ***p<0.001. Data are representative of 4-5 mice in each group.
(B) Apoptotic cell death is not influenced by BELMO expression. HCT116 cells were induced to undergo apoptosis by 3% DSS for 24 h. Staining with 7AAD and annexin V (AV) was used to determine the percentage of live (AV⁻7AAD⁻), apoptotic (AV⁺7AAD⁻) and necrotic (AV⁺7AAD⁺) cells. Data are representative of 3 experiments.
(C) Mice were treated with 3% DSS for 24 h. Colonic epithelial cell samples were collected, and tissue lysates prepared. Caspase-3 activity was determined by substrate cleavage activity kit (Caspase Glo 3/7). n.s. not significant. Data are representative of 4-5 mice in each group.

FIG. S4 BELMO ameliorates chemical-induced liver and kidney injuries, related to FIG. 4
(A, B) BELMO expression in hepatocytes reduces corpse accumulation in DEN-induced hepatocyte injury. BELMO^{Tg} mice were crossed with Alb-cre mice for hepatocyte expression of BELMO. Mice were treated with DEN for 48 h. Liver samples were collected, and caspase-3 activity determined by Caspase Glo 3/7 kit (A). Expression of cytokine genes were determined by qPCR (B). ***p<0.001. Data are representative of 4-9 mice in each group.
(C) Apoptosis of hepatocytes is not altered by BELMO expression. Control and BELMO expressing hepatocytes were induced to undergo apoptosis by staurosporine 1 μM for 12 h. Staining with 7AAD and annexin V (AV) was used to determine the percentage of live (AV⁻7AAD⁻), apoptotic (AV⁺7AAD⁻) or necrotic (AV⁺7AAD⁺) cells. Data are representative of 3 experiments.
(D) BELMO expression in kidney reduces corpse accumulation after cisplatin-induced kidney injury. BELMO^{Tg} mice were crossed with PEPCK-cre mice for TEC expression of BELMO. Mice were treated with cisplatin (20 mg/kg body weight) for 24 h. Kidney samples were collected, and cleaved caspase-3 was detected via immunohistochemistry. Scale bar 200 μm.
(E) BELMO expression in kidney does not inhibit initial injury induction by cisplatin. Mice were treated with cisplatin (20 mg/kg body weight) for 12 h. Plasma samples were collected and assessed for creatinine in circulation. n.s. not significant. Data are representative of 4 mice in each group.
(F) Mice were treated with cisplatin (20 mg/kg body weight) for 48 h. RNA was isolated from the kidneys, and the expression level of proinflammatory cytokines determined by qPCR (E). ***p<0.001. Data are representative of 4-9 mice in each group.
(G) CX3CR1-cre driven BELMO expression does not improve kidney function and viability. 48 h after cisplatin injection (20 mg/kg body weight), plasma samples were collected and assessed for creatinine in circulation. After cisplatin treatment (25 mg/kg body weight), the viability of mice was monitored over 7 days. Data are representative of three independent experiments with 3-10 mice in each group. n.s. not significant.

FIG. S5 Transcriptome analysis of BELMO expressing phagocytes, related to FIGS. 5 and 6
(A) Transcriptomic analysis of BELMO expressing macrophages during efferocytosis. BELMO or control peritoneal macrophages were co-cultured with apoptotic human Jurkat cells for 2 h. The unbound/free apoptotic cells were removed by washing, and LR73 cells were cultured for an additional 2 h. The mRNA from macrophages were then isolated and sequenced on the Illumina NextSeq platform and aligned with hamster or mouse-derived mRNA. GO enrichment analysis was used to identify gene programs modified in BELMO expressing cells. The bidirectional plots represent the normalized enrichment score. Hallmark gene sets are used in enrichment analysis. Protein folding/ER function gene sets are manually curated based on a public gene set (GO:0034976).
(B) BELMO or control LR73 fibroblasts were co-cultured with apoptotic human Jurkat cells for 2 h, the unbound/free apoptotic cells were removed by washing, and LR73 cells were cultured for an additional 2 h. The mRNA from LR73 cells was then isolated and the level of Bip, Dnajc3 and Vimp were determined by qPCR. ***p<0.001. **p<0.01. *p<0.05.
(C) CRISPR/Cas9 targeting of Bip, Dnajc3, Atp2a3 or Vimp in LR73 cells. Gene-targeted or control Cells were co-cultured with CypHer5E-labeled apoptotic Jurkat cells for 2 h. Phagocytosis was measured via flow cytometry. ***p<0.001. Data are representative of at least four independent experiments with 2-3 replicates per condition.
(D) BELMO expression in the kidney does not affect initial injury induction after IRI. BELMO^{Tg} mice and PEPCK-cre mice underwent bilateral IRI injury by clamping the renal pedicles for 26 m. The clamps were then removed and the wound sutured after restoration of blood flow was visually observed. Plasma samples were collected after 6 h and assessed for creatinine in circulation. n.s. not significant. Data are representative of 4 mice in each group.

FIG. S6 Generation and validation of TELMO, related to FIG. 7
(A) Schematic of TELMO generation.
(B) Adeno-associated virus (AAV)-mediated delivery ameliorates chronic kidney disease induced by ischemia-reperfusion. Left kidney was clamped and simultaneously AAV9-TELMO-GFP or control virus was introduced via renal vein injection; 25 min later, the clamp was removed. 14 days later, the contralateral right kidney was removed, and the left kidney function was evaluated after 24 h. Blood samples were collected for BUN quantification. Data are representative of at least three independent experiments with 5 mice per condition. ***p<0.001.

FIG. S7
(A) LR73 cells expressing BAI1 or BELMO were co-cultured with CypHer5E-labeled apoptotic Jurkat cells for 2 h. Phagocytosis was measured by quantification of CypHer5E+ cells by flow cytometry. Data are shown as % engulfment. ***p<0.001. Expression of the genes were shown by immunoblotting. n=4.
(B) LR73 cells expressing BAI1(1-1228)-FRB and ELMO1(532-727)-FKBP were treated with 10 μM rapamycin 30 min prior to the engulfment assay. Cells were co-cultured with CypHer5E-labeled apoptotic Jurkat cells for 2 h. Phagocytosis was measured by quantification of CypHer5E+ cells by flow cytometry. Data are shown as % engulfment. ***p<0.001. n=4.

### DETAILED DESCRIPTION OF THE INVENTION

A novel strategy to boost efferocytosis in vitro and in vivo was designed and denoted ‘chimeric receptor for efferocytosis’ (CHEF). The engineering of CHEF involves fusing a particular signaling domain within the cytoplasmic adapter ELMO1 to the extracellular phosphatidylserine recognition domains of the efferocytic receptors BAI1 or TIM4, generating BELMO and TELMO, respectively. BELMO and TELMO expressing phagocytes displayed a striking increase in efferocytosis of apoptotic cells. Transgenic mice and transgenic zebrafish with tissue or cell type-specific expression of BELMO, targeting different professional and non-professional phagocytes, potently boosted efferocytosis in vivo. In three different mouse models of inflammation, BELMO expression attenuated colitis in the gut, hepatotoxicity in the liver, and nephrotoxicity in the kidney. Mechanistic studies on BELMO identified protein-folding and misfolding/proteostasis in phagocytes as a rate-limiting step for enhancing efferocytosis. Specifically, BELMO increased the expression of ER-resident enzymes and chaperones to overcome proteotoxicity, and this was validated in ER-stress-induced renal ischemia-reperfusion injury in vivo. Finally, TELMO expression during ongoing kidney injury could significantly reduce fibrosis. Collectively, these data advance the concept of using chimeric efferocytic receptors to boost efferocytosis and dampen inflammation in vivo.

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14th Edition, by R. J. Lewis, John Wiley & Sons, New York, N.Y., 2001.

References in the specification to “one embodiment,” “an embodiment,” etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrase “one or more” is readily understood by one of skill in the art, particularly when read in context of its usage. For example, one or more substituents on a phenyl ring refers to one to five, or one to four, for example if the phenyl ring is di-substituted.

As used herein, “or” should be understood to have the same meaning as “and/or” as defined above. For example, when separating a listing of items, “and/or” or “or” shall be interpreted as being inclusive, e.g., the inclusion of at least one, but also including more than one of a number of items, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of”

As used herein, the terms “including,” “includes,” “having,” “has,” “with,” or variants thereof, are intended to be inclusive similar to the term “comprising.”

The term “about” can refer to a variation of ±5%, ±10%, ±20%, or ±25% of the value specified. For example, “about 50” percent can in some embodiments carry a variation from 45 to 55 percent. For integer ranges, the term “about” can include one or two integers greater than and/or less than a recited integer at each end of the range. Unless indicated otherwise herein, the term “about” is intended to include values, e.g., weight percentages, proximate to the recited range that are equivalent in terms of the functionality of the individual ingredient, the composition, or the embodiment. The term about can also modify the endpoints of a recited range as discuss above in this paragraph.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to,” “at least,” “greater than,” “less than,” “more than,” “or more,” and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents.

One skilled in the art will also readily recognize that where members are grouped together in a common manner, such as in a Markush group, the invention encompasses not only the entire group listed as a whole, but each member of the group individually and all possible subgroups of the main group.

Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

An “effective amount” refers to an amount effective to treat a disease, disorder, and/or condition, or to bring about a recited effect. For example, an effective amount can be an amount effective to reduce the progression or severity of the condition or symptoms being treated. Determination of a therapeutically effective amount is well within the capacity of persons skilled in the art, especially in light of the detailed disclosure provided herein. The term “effective amount” is intended to include an amount of a compound described herein, or an amount of a combination of compounds described herein, e.g., that is effective to treat or prevent a disease or disorder, or to treat the symptoms of the disease or disorder, in a host. Thus, an “effective amount” generally means an amount that provides the desired effect.

The terms “treating,” “treat” and “treatment” include (i) preventing a disease, pathologic or medical condition from occurring (e.g., prophylaxis); (ii) inhibiting the disease, pathologic or medical condition or arresting its development; (iii) relieving the disease, pathologic or medical condition; and/or (iv) diminishing symptoms associated with the disease, pathologic or medical condition. Thus, the terms “treat”, “treatment”, and “treating” can extend to prophylaxis and can include prevent, prevention, preventing, lowering, stopping or reversing the progression or severity of the condition or symptoms being treated. As such, the term “treatment” can include medical, therapeutic, and/or prophylactic administration, as appropriate.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

“Complementary” as used herein refers to the broad concept of subunit sequence complementarity between two nucleic acids, e.g., two DNA molecules. When a nucleotide position in both of the molecules is occupied by nucleotides normally capable of base pairing with each other, then the nucleic acids are considered to be complementary to each other at this position. Thus, two nucleic acids are complementary to each other when a substantial number (at least 50%) of corresponding positions in each of the molecules are occupied by nucleotides which normally base pair with each other (e.g., A:T and G:C nucleotide pairs). Thus, it is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. In one embodiment, the first region comprises a first portion and the second region comprises a second portion, whereby, when the first and second portions are arranged in an antiparallel fashion, at least about 50%, including at least about 75%, at least about 90%, or at least about 95% of the nucleotide residues of the first portion are capable of base pairing with nucleotide residues in the second portion. In some embodiments, all nucleotide residues of the first portion are capable of base pairing with nucleotide residues in the second portion.

A “compound,” as used herein, refers to any type of substance or agent that is commonly considered a drug, or a candidate for use as a drug, as well as combinations and mixtures of the above.

A “control” cell is a cell having the same cell type as a test cell. The control cell may, for example, be examined at precisely or nearly the same time the test cell is examined. The control cell may also, for example, be examined at a time distant from the time at which the test cell is examined, and the results of the examination of the control cell may be recorded so that the recorded results may be compared with results obtained by examination of a test cell.

The use of the word “detect” and its grammatical variants refers to measurement of the species without quantification, whereas use of the word “determine” or “measure” with their grammatical variants are meant to refer to measurement of the species with quantification. The terms “detect” and “identify” are used interchangeably herein.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCC5′ and 3′TATGGC5′ share 50% homology.

As used herein, “homology” is used synonymously with “identity.”

The determination of percent identity between two nucleotide or amino acid sequences can be accomplished using a mathematical algorithm. For example, a mathematical algorithm useful for comparing two sequences is the algorithm of Karlin and Altschul (1990, Proc. Natl. Acad. Sci. USA 87:2264-2268), modified as in Karlin and Altschul (1993, Proc. Natl. Acad. Sci. USA 90:5873-5877). This algorithm is incorporated into the NBLAST and XBLAST programs of Altschul, et al. (1990, J. Mol. Biol. 215:403-410), and can be accessed, for example at the National Center for Biotechnology Information (NCBI) world wide web site having the universal resource locator using the BLAST tool at the NCBI website. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al. (1997, Nucleic Acids Res. 25:3389-3402). Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted.

As used herein, the term “hybridization” is used in reference to the pairing of complementary nucleic acids. Hybridization and the strength of hybridization (i.e., the strength of the association between the nucleic acids) is impacted by such factors as the degree of complementarity between the nucleic acids, stringency of the conditions involved, the length of the formed hybrid, and the G:C ratio within the nucleic acids.

As used herein, an “instructional material” includes a publication, a recording, a diagram, or any other medium of expression which can be used to communicate the usefulness of the peptide/protein of the invention in the kit for effecting alleviation of the various diseases or disorders recited herein. Optionally, or alternately, the instructional material may describe one or more methods of alleviating the diseases or disorders in a cell or a tissue of a mammal. The instructional material of the kit of the invention may, for example, be affixed to a container which contains the identified compound invention or be shipped together with a container which contains the identified compound. Alternatively, the instructional material may be shipped separately from the container with the intention that the instructional material and the compound be used cooperatively by the recipient.

The term “nucleic acid” typically refers to large polynucleotides. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil).

As used herein, the term “nucleic acid” encompasses RNA as well as single and double-stranded DNA and cDNA. Furthermore, the terms, “nucleic acid,” “DNA,” “RNA” and similar terms also include nucleic acid analogs, i.e., analogs having other than a phosphodiester backbone. For example, the so-called “peptide nucleic acids,” which are known in the art and have peptide bonds instead of phosphodiester bonds in the backbone, are considered within the scope of the present invention. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil). Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

The term “nucleic acid construct,” as used herein, encompasses DNA and RNA sequences encoding the particular gene or gene fragment desired, whether obtained by genomic or synthetic methods.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “oligonucleotide” typically refers to short polynucleotides, generally, no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

As used herein, the term “pharmaceutically-acceptable carrier” means a chemical composition with which an appropriate compound or derivative can be combined and which, following the combination, can be used to administer the appropriate compound to a subject. “Pharmaceutically acceptable” means physiologically tolerable, for either human or veterinary application. As used herein, “pharmaceutical compositions” include formulations for human and veterinary use.

As used herein, “protecting group” with respect to a terminal amino group refers to a terminal amino group of a peptide/protein, which terminal amino group is coupled with any of various amino-terminal protecting groups traditionally employed in peptide synthesis. Such protecting groups include, for example, acyl protecting groups such as formyl, acetyl, benzoyl, trifluoroacetyl, succinyl, and methoxysuccinyl; aromatic urethane protecting groups such as benzyloxycarbonyl; and aliphatic urethane protecting groups, for example, tert-butoxycarbonyl or adamantyloxycarbonyl. See Gross and Mienhofer, eds., The Peptides, vol. 3, pp. 3-88 (Academic Press, New York, 1981) for suitable protecting groups.

As used herein, “protecting group” with respect to a terminal carboxy group refers to a terminal carboxyl group of a peptide/protein, which terminal carboxyl group is coupled with any of various carboxyl-terminal protecting groups. Such protecting groups include, for example, tert-butyl, benzyl or other acceptable groups linked to the terminal carboxyl group through an ester or ether bond.

“Recombinant polynucleotide” refers to a polynucleotide having sequences that are not naturally joined together. An amplified or assembled recombinant polynucleotide may be included in a suitable vector, and the vector can be used to transform a suitable host cell.

A recombinant polynucleotide may serve a non-coding function (e.g., promoter, origin of replication, ribosome-binding site, etc.) as well.

A host cell that comprises a recombinant polynucleotide is referred to as a “recombinant host cell.” A gene which is expressed in a recombinant host cell wherein the gene comprises a recombinant polynucleotide, produces a “recombinant polypeptide.”

A “recombinant polypeptide” or protein is one which is produced upon expression of a recombinant polynucleotide.

A “recombinant cell” is a cell that comprises a transgene. Such a cell may be a eukaryotic or a prokaryotic cell. Also, the transgenic cell encompasses, but is not limited to, an embryonic stem cell comprising the transgene, a cell obtained from a chimeric mammal derived from a transgenic embryonic stem cell where the cell comprises the transgene, a cell obtained from a transgenic mammal, or fetal or placental tissue thereof, and a prokaryotic cell comprising the transgene.

The term “regulate” refers to either stimulating or inhibiting a function or activity of interest.

The term “standard,” as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

As used herein, a “subject in need thereof” is a patient, animal, mammal, or human, who will benefit from the method of this invention.

As used herein, a “substantially homologous amino acid sequences” includes those amino acid sequences which have at least about 90% homology, at least about 95% homology, at least about 96% homology, at least about 97% homology, at least about 98% homology, or at least about 99% or more homology to an amino acid sequence of a reference antibody chain. Amino acid sequence similarity or identity can be computed by using the BLASTP and TBLASTN programs which employ the BLAST (basic local alignment search tool) 2.0.14 algorithm. The default settings used for these programs are suitable for identifying substantially similar amino acid sequences for purposes of the present invention.

“Substantially homologous nucleic acid sequence” means a nucleic acid sequence corresponding to a reference nucleic acid sequence wherein the corresponding sequence encodes a peptide having substantially the same structure and function as the peptide encoded by the reference nucleic acid sequence; e.g., where only changes in amino acids not significantly affecting the peptide function occur. In an example, the substantially identical nucleic acid sequence encodes the peptide encoded by the reference nucleic acid sequence. The percentage of identity between the substantially similar nucleic acid sequence and the reference nucleic acid sequence (or the amino acid sequence and the reference amino acid sequence) is at least about 50%, 60% 65%, 70%, 75%, 80%, 85%, 90%, 95%, 99% or more. Substantial identity of nucleic acid sequences can be determined by comparing the sequence identity of two sequences, for example by physical/chemical methods (i.e., hybridization) or by sequence alignment via computer algorithm. Suitable nucleic acid hybridization conditions to determine if a nucleotide sequence is substantially similar to a reference nucleotide sequence are: 7% sodium dodecyl sulfate SDS, 0.5 M NaPO₄, 1 mM EDTA at 50° C. with washing in 2× standard saline citrate (SSC), 0.1% SDS at 50° C.; preferably in 7% (SDS), 0.5 M NaPO₄, 1 mM EDTA at 50° C. with washing in 1×SSC, 0.1% SDS at 50° C.; preferably 7% SDS, 0.5 M NaPO₄, 1 mM EDTA at 50° C. with washing in 0.5×SSC, 0.1% SDS at 50° C.; and more preferably in 7% SDS, 0.5 M NaPO₄, 1 mM EDTA at 50° C. with washing in 0.1×SSC, 0.1% SDS at 65° C. Suitable computer algorithms to determine substantial similarity between two nucleic acid sequences include, GCS program package (Devereux et al., 1984 Nucl. Acids Res. 12:387), and the BLASTN or FASTA programs (Altschul et al., 1990 Proc. Natl. Acad. Sci. USA. 1990 87:14:5509-13; Altschul et al., J. Mol. Biol. 1990 215:3:403-10; Altschul et al., 1997 Nucleic Acids Res. 25:3389-3402). The default settings provided with these programs are suitable for determining substantial similarity of nucleic acid sequences for purposes of the present invention.

The term “symptom,” as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse and other observers.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer or delivery of nucleic acid to cells, such as, for example, polylysine compounds, liposomes, and the like. Examples of viral vectors include, but are not limited to, adenoviral vectors, adeno-associated virus vectors, retroviral vectors, recombinant viral vectors, and the like. Examples of non-viral vectors include, but are not limited to, liposomes, polyamine derivatives of DNA and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses that incorporate the recombinant polynucleotide.

Methods involving conventional molecular biology techniques are described herein. Such techniques are generally known in the art and are described in detail in methodology treatises, such as Molecular Cloning: A Laboratory Manual, 2nd ed., vol. 1-3, ed. Sambrook et al., Cold Spring Harbor Laboratory Press, Cold Spring Harbor, N.Y., 1989; and Current Protocols in Molecular Biology, ed. Ausubel et al., Greene Publishing and Wiley-Interscience, New York, 1992 (with periodic updates). Methods for chemical synthesis of nucleic acids are discussed, for example, in Beaucage and Carruthers, Tetra. Letts. 22: 1859-1862, 1981, and Matteucci et al., J. Am. Chem. Soc. 103:3185, 1981.

As used herein, amino acids are represented by the full name thereof, by the three-letter code corresponding thereto, or by the one-letter code corresponding thereto, as indicated in the following table:

| | Three-Letter | One-Letter |
|---|---|---|
| Full Name | Code | Code |
| Aspartic Acid | Asp | D |
| Glutamic Acid | Glu | E |
| Lysine | Lys | K |
| Arginine | Arg | R |
| Histidine | His | H |
| Tyrosine | Tyr | Y |
| Cysteine | Cys | C |
| Asparagine | Asn | N |
| Glutamine | Gln | Q |
| Serine | Ser | S |
| Threonine | Thr | T |
| Glycine | Gly | G |
| Alanine | Ala | A |
| Valine | Val | V |
| Leucine | Leu | L |
| Isoleucine | Ile | I |
| Methionine | Met | M |
| Proline | Pro | P |
| Phenylalanine | Phe | F |
| Tryptophan | Trp | W |

The term “amino acid” is used interchangeably with “amino acid residue,” and may refer to a free amino acid and to an amino acid residue of a peptide/protein. It will be apparent from the context in which the term is used whether it refers to a free amino acid or a residue of a peptide/protein.

The expression “amino acid” as used herein is meant to include both natural and synthetic amino acids, and both D and L amino acids. “Standard amino acid” means any of the twenty standard L-amino acids commonly found in naturally occurring peptides/proteins. “Nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides/proteins of the present invention, and particularly at the carboxy- or amino-terminus, can be modified by methylation, amidation, acetylation or substitution with other chemical groups which can change the peptide's/protein's circulating half-life without adversely affecting their activity (e.g., peptidomimetic for making peptides protease resistant).

Amino acids have the following general structure:

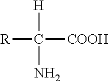

Amino acids may be classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group.

The nomenclature used to describe the peptide/protein compounds of the present invention follows the conventional practice wherein the amino group is presented to the left and the carboxy group to the right of each amino acid residue. In the formulae representing selected specific embodiments of the present invention, the amino- and carboxy-terminal groups, although not specifically shown, will be understood to be in the form they would assume at physiologic pH values, unless otherwise specified.

The term “basic” or “positively charged” amino acid as used herein, refers to amino acids in which the R groups have a net positive charge at pH 7.0, and include, but are not limited to, the standard amino acids lysine, arginine, and histidine.

As used herein, the term “conservative amino acid substitution” is defined herein as an amino acid exchange within one of the following five groups:
I. Small aliphatic, nonpolar or slightly polar residues:
   Ala, Ser, Thr, Pro, Gly;
II. Polar, negatively charged residues and their amides:
   Asp, Asn, Glu, Gln;
III. Polar, positively charged residues:
   His, Arg, Lys;
IV. Large, aliphatic, nonpolar residues:
   Met Leu, Ile, Val, Cys
V. Large, aromatic residues:
   Phe, Tyr, Trp.

### Apoptosis/Efferocytosis

The clearance of apoptotic cells by phagocytes, a process termed efferocytosis, is essential for maintaining tissue homeostasis (Boada-Romero et al., 2020; Doran et al., 2020; Elliott and Ravichandran, 2016; Gregory and Pound, 2010). 200-300 billion cells die by apoptosis each day and are cleared via efferocytosis without an inflammatory consequence. This efferocytic process is carried out by professional phagocytes, such as macrophages, as well as non-professional phagocytes, including epithelial cells, fibroblasts, and endothelial cells (Boada-Romero et al., 2020; Doran et al., 2020; Elliott and Ravichandran, 2016; Gregory and Pound, 2010; Han et al., 2016; Juncadella et al., 2013; Lemke, 2019; Monks et al., 2008; Morioka et al., 2019; Shankman et al., 2021). As apoptosis can increase locally under pathological conditions, including tissue injury, infection, neurodegenerative diseases, and autoimmune diseases, apoptotic cells can accumulate; in such cases, uncleared apoptotic cells can advance to secondary necrosis and in turn, exacerbate inflammation and pathology. Thus, defective efferocytosis is considered a contributing factor to a growing list of human diseases, including atherosclerosis, systemic lupus erythematosus and atherosclerosis, and ulcerative colitis (Boada-Romero et al., 2020; Doran et al., 2020; Elliott and Ravichandran, 2016; Gregory and Pound, 2010; Henson, 2017; Henson and Hume, 2006; Lemke, 2019; Morioka et al., 2019; Nagata, 2018; Rothlin et al., 2021)

### Cytoplasmic Adapter/ELMO

ELMO associates with Dock180, and the ELMO/Dock complex functions as a guanine nucleotide exchange factor for the small GTPase Rac1 to promote cytoskeletal reorganization during the engulfment of apoptotic cells (Brugnera et al., 2002; Gumienny et al., 2001; Park et al., 2007). The Dock/ELMO/Rac module is highly conserved in evolution and regulates efferocytosis in C. elegans, drosophila, zebrafish, and mammals (Elliott et al., 2010; Epting et al., 2010; Geisbrecht et al., 2008; Gumienny et al., 2001).

Elmo1 (75 kDa) is a cytoplasmic adapter protein that physically associates with members of the Dock-A family of Rac-GEFs, such as Dock1 and Dock2. Structure-function analyses have shown that Elmo binding enhances Dock1 signaling by increasing its Rac-GEF activity, membrane localization and protein stability. Studies in invertebrate models and mammalian cell lines have revealed an evolutionarily conserved role for Elmo1 in regulating Dock-Rac signaling in numerous cellular functions, including morphology, motility and phagocytosis.

Elmo1 and Elmo2 are 87% similar at the amino acid level, are widely expressed and may functionally redundant (13). Both proteins contain pleckstrin homology (PH) and proline-rich/PxxP domains located in the C-terminal 100aa. These C-terminal regions mediate multiple associations with the N-termini of Dock1 and Dock2 as revealed through crystallographic and biochemical analyses. Dock1 and Dock2 contain an N-terminal Src homology 3 (SH3) domain that mediates interaction with the C-terminal polyproline regions of Elmo1 and Elmo2. Interestingly, this PxxP-SH3 association is needed for Elmo1 interaction with Dock2 but not Dock1 (31). The PxxP motif is conserved between mouse and human Elmo1 and Elmo2 (PKEP, Elmo1714-717).

Homo sapiens ELMO1 mRNA, complete cds

| (SEQ ID NO: 21) | |
|---|---|
| MPPPADIVKVAIEWPGAYPKLMEIDQKKPLSAIIKEVCDGWSLA | |
| NHEYFALQHADSSNFYITEKNRNEIKNGTILRLTTSPAQNAQQLHERIQSSSMDAKLE | |
| ALKDLASLSRDVTFAQEFINLDGISLLTQMVESGTERYQKLQKIMKPCFGDMLSFTLT | |
| AFVELMDHGIVSWDTFSVAFIKKIASFVNKSAIDISILQRSLAILESMVLNSHDLYQK | |
| VAQEITIGQLIPHLQGSDQEIQTYTIAVINALFLKAPDERRQEMANILAQKQLRSIIL | |
| THVIRAQRAINNEMAHQLYVLQVLTENLLEDRMMTKMDPQDQAQRDIIFELRRIAFDA | |
| ESEPNNSSGSMEKRKSMYTRDYKKLGFINHVNPAMDFTQTPPGMLALDNMLYFAKHHQ | |
| DAYIRIVLENSSREDKHECPFGRSSIELTKMLCEILKVGELPSETCNDFHPMFFTHDR | |
| SFEEFFCICIQLLNKTWKEMRATSEDFNKVMQVVKEQVMRALTTKPSSLDQFKSKLQN | |
| LSYTEILKIRQSERMNQEDFQSRPILELKEKIQPEILELIKQQRLNRLVEGTCFRKLN | |
| ARRRQDKFWYCRLSPNHKVLHYGDLEESPQGEVPHDSLQDKLPVADIKAVVTGKDCPH | |
| MKEKGALKQNKEVLELAFSILYDSNCQLNFIAPDKHEYCIWTDGLNALLGKDMMSDLT | |
| RNDLDTLLSMEIKLRLLDLENIQIPDAPPPIPKEPSNYDFVYDCN | |
| (SEQ ID NO: 22) | |
| 1 | caatgccgcc acccgcggac atcgtcaagg tggccataga atggccgggc gcctacccca |
| 61 | aactcatgga aattgatcag aaaaaaccac tgtctgcaat aataaaggaa gtctgtgatg |
| 121 | ggtggtctct tgccaaccat gaatattttg cactccagca tgccgatagt tcaaacttct |
| 181 | atatcacaga aaagaaccgc aatgagataa aaaatggcac tatccttcga ttaaccacat |
| 241 | ctccagctca gaacgcccag cagctccatg aacgaatcca gtcctcgagt atggatgcca |
| 301 | agctggaagc cctgaaggac ttggccagcc tctcccggga tgtcacgttt gcccaggagt |
| 361 | ttataaacct ggacggtatc tctctcctca cgcagatggt ggagagcggc actgagcgat |
| 421 | accagaaatt gcagaagatc atgaagcctt gctttggaga catgctgtcc ttcaccctga |
| 481 | cggccttcgt cgagctgatg gaccatggca tagtgtcctg ggatacattt tcggtggcgt |
| 541 | tcattaagaa gatagcaagt tttgtgaaca agtcagccat agacatctcg atcctgcagc |
| 601 | ggtccttggc cattttggag tcgatggtgc tcaatagcca tgatctctac cagaaagtgg |
| 661 | cgcaggagat caccatcggc cagctcattc cacacctgca agggtcagat caagaaatcc |
| 721 | aaacctatac tattgcagtg attaatgcgc ttttcctgaa ggctcctgat gagaggaggc |
| 781 | aggagatggc gaatattttg gctcagaagc aactgcgttc catcatttta acacatgtca |
| 841 | tccgagccca gcgggccatc aacaatgaga tggcgcacca gctgtatgtt ctacaagtgc |
| 901 | tcacctttaa cctcctggaa gacaggatga tgaccaaaat ggacccccag gaccaggctc |
| 961 | agagggacat catatttgaa cttcgaagaa ttgcttttga tgctgagtct gaacctaaca |
| 1021 | acagcagtgg cagcatggag aaacgcaagt ccatgtacac gcgagattat aagaagcttg |
| 1081 | ggttcattaa tcatgtcaac cctgccatgg acttcacgca gactccacct gggatgttgg |
| 1141 | ctctggacaa catgctgtac tttgccaagc accaccaaga tgcctacatc cggattgtgc |
| 1201 | ttgagaacag tagtcgagaa gacaagcatg aatgtccctt tggccgcagt agtatagagc |
| 1261 | tgaccaagat gctatgtgag atcttgaaag tgggcgagtt gcctagtgag acctgcaacg |
| 1321 | acttccaccc gatgttcttc acccacgaca gatcctttga ggagtttttc tgcatctgta |
| 1381 | tccagctcct gaacaagaca tggaaggaaa tgagggcaac ttctgaagac ttcaacaagg |
| 1441 | taatgcaggt ggtgaaggag caggttatga gagcacttac aaccaagcct agctccctgg |
| 1501 | accagttcaa gagcaaactg cagaacctga gctacactga gatcctgaaa atccgccagt |
| 1561 | ccgagaggat gaaccaggaa gatttccagt cccgcccgat tttggaacta aaggagaaga |
| 1621 | ttcagccaga aatcttagag ctgatcaaac agcaacgcct gaaccgcctt gtggaaggga |
| 1681 | cctgctttag gaaactcaat gcccggcgga ggcaagacaa gttttggtat tgtcggcttt |
| 1741 | cgccaaatca caaagtcctg cattacggag acttagaaga gagtcctcag ggagaagtgc |
| 1801 | cccacgattc cttgcaggac aaactgccgg tggcagatat caaagccgtg gtgacgggaa |
| 1861 | aggactgccc tcatatgaaa gagaaaggtg cccttaaaca aaacaaggag gtgcttgaac |
| 1921 | tcgctttctc catcttgtat gactcaaact gccaactgaa cttcatcgct cctgacaagc |
| 1981 | atgagtactg tatctggacg gatggactga atgcgctact cgggaaggac atgatgagcg |
| 2041 | acctgacgcg gaatgacctg gacaccctgc tcagcatgga aatcaagctc cgcctcctgg |
| 2101 | acctggaaaa catccagatc cctgacgcac ctccgccgat tcccaaggag cccagcaact |
| 2161 | atgacttcgt ctatgactgt aactga |

### Extracellular Phosphatidylserine Recognition Domain of Efferocytic Receptors

In terms of recognizing and removing apoptotic cells, the exposure of phosphatidylserine (PtdSer) on apoptotic cells plays a role. After apoptosis induction, PtdSer is translocated from the inner to the outer leaflet of the plasma membrane, and several phagocytic receptors on phagocytes have been identified to engage PtdSer through direct or indirect interactions (Boada-Romero et al., 2020; Doran et al., 2020; Gregory and Pound, 2010; Morioka et al., 2019; Nagata, 2018; Rothlin et al., 2021). Integrins (αvβ and αvβ5) and the Tyro3/Axl/Mer (TAM) family of tyrosine kinase receptors recognize apoptotic cells indirectly through association with secreted PtdSer-binding proteins MFG E8 and Gas6/Protein S, respectively (Boada-Romero et al., 2020; Doran et al., 2020; Elliott and Ravichandran, 2016; Gregory and Pound, 2010; Morioka et al., 2019; Nagata, 2018; Rothlin et al., 2021).

### TIM4

Tim4 is a phosphatidylserine (PS) receptor that is expressed on various macrophage subsets. It mediates phagocytosis of apoptotic cells by peritoneal macrophages. Among the direct PtdSer recognition receptors, TIM4 binds PtdSer directly, but due to the lack of an intracellular signaling domain, it acts as a tethering receptor (Nagata, 2018; Park et al., 2009). Homo sapiens T cell immunoglobulin and mucin domain containing 4 (TIMD4), transcript variant 1, mRNA

| (SEQ ID NO: 23) | |
|---|---|
| MSKEPLILWLMIEFWWLYLTPVTSETVVTEVLGHRVTLPCLYSS | |
| WSHNSNSMCWGKDQCPYSGCKEALIRTDGMRVTSRKSAKYRLQGTIPRGDVSLTILNP | |
| SESDSGVYCCRIEVPGWFNDVKINVRLNLQRASTTTHRTATTTTRRTTTTSPTTTRQM | |
| TTTPAALPTTVVTTPDLTTGTPLQMTTIAVFTTANTCLSLTPSTLPEEATGLLTPEPS | |
| KEGPILTAESETVLPSDSWSSVESTSADTVLLTSKESKVWDLPSTSHVSMWKTSDSVS | |
| SPQPGASDTAVPEQNKTTKTGQMDGIPMSMKNEMPISQLLMIIAPSLGFVLFALFVAF | |
| LLRGKLMETYCSQKHTRLDYIGDSKNVLNDVQHGREDEDGLFTL | |
| (SEQ ID NO: 24) | |
| 1 | agactcctgg gtccggtcaa ccgtcaaaat gtccaaagaa cctctcattc tctggctgat |
| 61 | gattgagttt tggtggcttt acctgacacc agtcacttca gagactgttg tgacggaggt |
| 121 | tttgggtcac cgggtgactt tgccctgtct gtactcatcc tggtctcaca acagcaacag |
| 181 | catgtgctgg gggaaagacc agtgccccta ctccggttgc aaggaggcgc tcatccgcac |
| 241 | tgatggaatg agggtgacct caagaaagtc agcaaaatat agacttcagg ggactatccc |
| 301 | gagaggtgat gtctccttga ccatcttaaa ccccagtgaa agtgacagcg gtgtgtactg |
| 361 | ctgccgcata gaagtgcctg gctggttcaa cgatgtaaag ataaacgtgc gcctgaatct |
| 421 | acagagagcc tcaacaacca cgcacagaac agcaaccacc accacacgca gaacaacaac |
| 481 | aacaagcccc accaccaccc gacaaatgac aacaacccca gctgcacttc caacaacagt |
| 541 | cgtgaccaca cccgatctca caaccggaac accactccag atgacaacca ttgccgtctt |
| 601 | cacaacagca aacacgtgcc tttcactaac cccaagcacc cttccggagg aagccacagg |
| 661 | tcttctgact cccgagcctt ctaaggaagg gcccatcctc actgcagaat cagaaactgt |
| 721 | cctccccagt gattcctgga gtagtgttga gtctacttct gctgacactg tcctgctgac |
| 781 | atccaaagag tccaaagttt gggatctccc atcaacatcc cacgtgtcaa tgtggaaaac |
| 841 | gagtgattct gtgtcttctc ctcagcctgg agcatctgat acagcagttc ctgagcagaa |
| 901 | caaaacaaca aaaacaggac agatggatgg aatacccatg tcaatgaaga atgaaatgcc |
| 961 | catctcccaa ctactgatga tcatcgcccc ctccttggga tttgtgctct tcgcattgtt |
| 1021 | tgtggcgttt ctcctgagag ggaaactcat ggaaacctat tgttcgcaga aacacacaag |
| 1081 | gctagactac attggagata gtaaaaatgt cctcaatgac gtgcagcatg gaagggaaga |
| 1141 | cgaagacggc ctttttaccc tctaacaacg cagtagcatg ttagattgag gatgggggca |
| 1201 | tgacactcca gtgtcaaaat aagtcttagt agatttcctt gtttcataaa aaagactcac |
| 1261 | ttattccatg gatgtcattg atccaggctt gctttagttt catgaatgaa gggtacttta |
| 1321 | gagaccacaa |

Homo sapiens T cell immunoglobulin and mucin domain containing 4 (TIMD4), transcript variant 2, mRNA

| (SEQ ID NO: 25) | |
|---|---|
| MSKEPLILWLMIEFWWLYLTPVTSETVVTEVLGHRVTLPCLYSS | |
| WSHNSNSMCWGKDQCPYSGCKEALIRTDGMRVTSRKSAKYRLQGTIPRGDVSLTILNP | |
| SESDSGVYCCRIEVPGWFNDVKINVRLNLQRASTTTHRTATTTTRRTTTTSPTTTRQM | |
| TTTPAALPTTVVTTPDLTTGTPLQMTTIAVFTTANTCLSLTPSTLPEEATGLLTPEPS | |
| KEGPILTAESETVLPSDSWSSVESTSADTVLLTSKASDTAVPEQNKTTKTGQMDGIPM | |
| SMKNEMPISQLLMIIAPSLGFVLFALFVAFLLRGKLMETYCSQKHTRLDYIGDSKNVL | |
| NDVQHGREDEDGLFTL | |
| (SEQ ID NO: 26) | |
| 1 | agactcctgg gtccggtcaa ccgtcaaaat gtccaaagaa cctctcattc tctggctgat |
| 61 | gattgagttt tggtggcttt acctgacacc agtcacttca gagactgttg tgacggaggt |
| 121 | tttgggtcac cgggtgactt tgccctgtct gtactcatcc tggtctcaca acagcaacag |
| 181 | catgtgctgg gggaaagacc agtgccccta ctccggttgc aaggaggcgc tcatccgcac |
| 241 | tgatggaatg agggtgacct caagaaagtc agcaaaatat agacttcagg ggactatccc |
| 301 | gagaggtgat gtctccttga ccatcttaaa ccccagtgaa agtgacagcg gtgtgtactg |
| 361 | ctgccgcata gaagtgcctg gctggttcaa cgatgtaaag ataaacgtgc gcctgaatct |
| 421 | acagagagcc tcaacaacca cgcacagaac agcaaccacc accacacgca gaacaacaac |
| 481 | aacaagcccc accaccaccc gacaaatgac aacaacccca gctgcacttc caacaacagt |
| 541 | cgtgaccaca cccgatctca caaccggaac accactccag atgacaacca ttgccgtctt |
| 601 | cacaacagca aacacgtgcc tttcactaac cccaagcacc cttccggagg aagccacagg |
| 661 | tcttctgact cccgagcctt ctaaggaagg gcccatcctc actgcagaat cagaaactgt |
| 721 | cctccccagt gattcctgga gtagtgttga gtctacttct gctgacactg tcctgctgac |
| 781 | atccaaagca tctgatacag cagttcctga gcagaacaaa acaacaaaaa caggacagat |
| 841 | ggatggaata cccatgtcaa tgaagaatga aatgcccatc tcccaactac tgatgatcat |
| 901 | cgccccctcc ttgggatttg tgctcttcgc attgtttgtg gcgtttctcc tgagagggaa |
| 961 | actcatggaa acctattgtt cgcagaaaca cacaaggcta gactacattg gagatagtaa |
| 1021 | aaatgtcctc aatgacgtgc agcatggaag ggaagacgaa gacggccttt ttaccctcta |
| 1081 | acaacgcagt agcatgttag attgaggatg ggggcatgac actccagtgt caaaataagt |
| 1141 | cttagtagat ttccttgttt cataaaaaag actcacttat tccatggatg tcattgatcc |
| 1201 | aggcttgctt tagtttcatg aatgaagggt actttagaga ccacaa |

Homo sapiens T cell immunoglobulin and mucin domain containing 4 (TIMD4), transcript variant X2, mRNA

(SEQ ID NO: 27)
MSKEPLILWLMIEFWWLYLTPVTSETVVTEVLGHRVTLPCLYSS
WSHNSNSMCWGKDQCPYSGCKEALIRTDGMRVTSRKSAKYRLQGTIPRGDVSLTILNP
SESDSGVYCCRIEVPGWFNDVKINVRLNLQRASTTTHRTATTTTRRTTTTSPTTTRQM
TTTPAALPTTVVTTPDLTTGTPLQMTTIAVFTTANTCLSLTPSTLPEEATGLLTPEPS
KEGPILTAESETVLPSDSWSSVESTSADTVLLTSKESKVWDLPSTSHVSMWKTSDSVS
SPQPGEMSSHHVAQAGLKSLGLK

Homo sapiens T cell immunoglobulin and mucin domain containing 4 (TIMD4), transcript variant X1, mRNA

| (SEQ ID NO: 28) | |
|---|---|
| MSKEPLILWLMIEFWWLYLTPVTSETVVTEVLGHRVTLPCLYSS | |
| WSHNSNSMCWGKDQCPYSGCKEALIRTDGMRVTSRKSAKYRLQGTIPRGDVSLTILNP | |
| SESDSGVYCCRIEVPGWFNDVKINVRLNLQRASTTTHRTATTTTRRTTTTSPTTTRQM | |
| TTTPAALPTTVVTTPDLTTGTPLQMTTIAVFTTANTCLSLTPSTLPEEATGLLTPEPS | |
| KEGPILTAASDTAVPEQNKTTKTGQMDGIPMSMKNEMPISQLLMIIAPSLGFVLFALF | |
| VAFLLRGKLMETYCSQKHTRLDYIGDSKNVLNDVqHGREDEDGLFTL | |
| (SEQ ID NO: 29) | |
| 1 | agactcctgg gtccggtcaa ccgtcaaaat gtccaaagaa cctctcattc tctggctgat |
| 61 | gattgagttt tggtggcttt acctgacacc agtcacttca gagactgttg tgacggaggt |
| 121 | tttgggtcac cgggtgactt tgccctgtct gtactcatcc tggtctcaca acagcaacag |
| 181 | catgtgctgg gggaaagacc agtgccccta ctccggttgc aaggaggcgc tcatccgcac |
| 241 | tgatggaatg agggtgacct caagaaagtc agcaaaatat agacttcagg ggactatccc |
| 301 | gagaggtgat gtctccttga ccatcttaaa ccccagtgaa agtgacagcg gtgtgtactg |
| 361 | ctgccgcata gaagtgcctg gctggttcaa cgatgtaaag ataaacgtgc gcctgaatct |
| 421 | acagagagcc tcaacaacca cgcacagaac agcaaccacc accacacgca gaacaacaac |
| 481 | aacaagcccc accaccaccc gacaaatgac aacaacccca gctgcacttc caacaacagt |
| 541 | cgtgaccaca cccgatctca caaccggaac accactccag atgacaacca ttgccgtctt |
| 601 | cacaacagca aacacgtgcc tttcactaac cccaagcacc cttccggagg aagccacagg |
| 661 | tcttctgact cccgagcctt ctaaggaagg gcccatcctc actgcagcat ctgatacagc |
| 721 | agttcctgag cagaacaaaa caacaaaaac aggacagatg gatggaatac ccatgtcaat |
| 781 | gaagaatgaa atgcccatct cccaactact gatgatcatc gccccctcct tgggatttgt |
| 841 | gctcttcgca ttgtttgtgg cgtttctcct gagagggaaa ctcatggaaa cctattgttc |
| 901 | gcagaaacac acaaggctag actacattgg agatagtaaa aatgtcctca atgacgtgca |
| 961 | gcatggaagg gaagacgaag acggcctttt taccctctaa caacgcagta gcatgttaga |
| 1021 | ttgaggatgg gggcatgaca ctccagtgtc aaaataagtc ttagtagatt tccttgtttc |
| 1081 | ataaaaaaga ctcacttatt ccatggatgt cattgatcca ggcttgcttt agtttcatga |
| 1141 | atgaagggta ctttagagac cacaa |

### BAI1

BAI1 functions as an engulfment receptor in both the recognition and subsequent internalization of apoptotic cells. Phosphatidylserine, is an ‘eat-me’ signal exposed on apoptotic cells as a ligand for BAI1. As with intracellular signaling, BAI1 forms a trimeric complex with ELMO and Dock180, and functional studies suggest that BAI1 cooperates with ELMO/Dock180/Rac to promote engulfment of apoptotic cells. BAI1 is another direct PtdSer binding receptor and, via its cytoplasmic tail domain, interacts with the engulfment promoting cytoplasmic protein, ELMO.

Homo sapiens BAI 1 mRNA, complete cds

| (SEQ ID NO: 19) | |
|---|---|
| MRGQAAAPGPVWILAPLLLLLLLLGRRARAAAGADAGPGPEPCA | |
| TLVQGKFFGYFSAAAVFPANASRCSWTLRNPDPRRYTLYMKVAKAPVPCSGPGRVRTY | |
| QFDSFLESTRTYLGVESFDEVLRLCDPSAPLAFLQASKQFLQMRRQQPPQHDGLRPRA | |
| GPPGPTDDFSVEYLVVGNRNPSRAACQMLCRWLDACLAGSRSSHPCGIMQTPCACLGG | |
| EAGGPAAGPLAPRGDVCLRDAVAGGPENCLTSLTQDRGGHGATGGWKLWSLWGECTRD | |
| CGGGLQTRTRTCLPAPGVEGGGCEGVLEEGRQCNREACGPAGRTSSRSQSLRSTDARR | |
| REELGDELQQFGFPAPQTGDPAAEEWSPWSVCSSTCGEGWQTRTRFCVSSSYSTQCSG | |
| PLREQRLCNNSAVCPVHGAWDEWSPWSLCSSTCGRGFRDRTRTCRPPQFGGNPCEGPE | |
| KQTKFCNIALCPGRAVDGNWNEWSSWSACSASCSQGRQQRTRECNGPSYGGAECQGHW | |
| VETRDCFLQQCPVDGKWQAWASWGSCSVTCGAGSQRRERVCSGPFFGGAACQGPQDEY | |
| RQCGTQRCPEPHEICDEDNFGAVIWKETPAGEVAAVRCPRNATGLILRRCELDEEGIA | |
| YWEPPTYIRCVSIDYRNIQMMTREHLAKAQRGLPGEGVSEVIQTLVEISQDGTSYSGD | |
| LLSTIDVLRNMTEIFRRAYYSPTPGDVQNFVQILSNLLAEENRDKWEEAQLAGPNAKE | |
| LFRLVEDFVDVIGERMKDLRDAYQVTDNLVLSIHKLPASGATDISFPMKGWRATGDWA | |
| KVPEDRVTVSKSVFSTGLTEADEASVFVVGTVLYRNLGSFLALQRNTTVLNSKVISVT | |
| VKPPPRSLRTPLEIEFAHMYNGTTNQTCILWDETDVPSSSAPPQLGPWSWRGCRTVPL | |
| DALRTRCLCDRLSTFAILAQLSADANMEKATLPSVTLIVGCGVSSLTLLMLVIIYVSV | |
| WRYIRSERSVILINFCLSIISSNALILIGQTQTRNKVMCTLVAAFLHFFFLSSFCWVL | |
| TEAWQSYMAVTGHLRNRLIRKRFLCLGWGLPALVVAISVGFTKAKGYSTMNYCWLSLE | |
| GGLLYAFVGPAAAVVLVNMVIGILVFNKLVSKDGITDKKLKERAGASLWSSCVVLPLL | |
| ALTWMSAVLAVTDRRSALFQILFAVFDSLEGFVIVMVHCILRREVQDAVKCRVVDRQE | |
| EGNGDSGGSFQNGHAQLMTDFEKDVDLACRSVLNKDIAACRTATITGTLKRPSLPEEE | |
| KLKLAHAKGPPTNFNSLPANVSKLHLHGSPRYPGGPLPDFPNHSLTLKRDKAPKSSFV | |
| GDGDIFKKLDSELSRAQEKALDTSYVILPTATATLRPKPKEEPKYSIHIDQMPQTRLI | |
| HLSTAPEASLPARSPPSRQPPSGGPPEAPPAQPPPPPPPPPPPPQQPLPPPPNLEPAP | |
| PSLGDPGEPAAHPGPSTGPSTKNENVATLSVSSLERRKSRYAELDFEKIMHTRKRHQD | |
| MFQDLNRKLQHAAEKDKEVLGPDSKPEKQQTPNKRPWESLRKAHGTPTWVKKELEPLQ | |
| PSPLELRSVEWERSGATIPLVGQDIIDLQTEV | |
| (SEQ ID NO: 20) | |
| 1 | ggactttaga agccgttgct gccctctctg tcacctgaag cggggccctc tcccatccca |
| 61 | cccttgcccc gcctccctgc ccccaccggg ccggccctgc ccgccgccgg accctggcat |
| 121 | gtcaagacct ggtccgcgcc tgcctgccca gcccgcggaa ccccggcggc cccgcgagct |
| 181 | aggatgaggg gccaggccgc cgccccgggc cccgtctgga tcctcgcccc gctgctactg |
| 241 | ctgctgctgc tgctgggacg ccgcgcgcgg gcggccgccg gagcagacgc ggggcccggg |
| 301 | cccgagccgt gcgccacgct ggtgcaggga aagttcttcg gctacttctc cgcggccgcc |
| 361 | gtgttcccgg ccaacgcctc gcgctgctcc tggacgctac gcaacccgga cccgcggcgc |
| 421 | tacactctct acatgaaggt ggccaaggcg cccgtgccct gcagcggccc cggccgcgtg |
| 481 | cgcacctacc agttcgactc cttcctcgag tccacgcgca cctacctggg cgtggagagc |
| 541 | ttcgacgagg tgctgcggct ctgcgacccc tccgcacccc tggccttcct gcaggccagc |
| 601 | aagcagttcc tgcagatgcg gcgccagcag ccgccccagc acgacgggct ccggccccgg |
| 661 | gccgggccgc cgggccccac cgacgacttc tccgtggagt acctggtggt ggggaaccgc |
| 721 | aaccccagcc gtgccgcctg ccagatgctg tgccgctggc tggacgcgtg tctggccggt |
| 781 | agtcgcagct cgcacccctg cgggatcatg cagaccccct gcgcctgcct gggcggcgag |
| 841 | gcgggcggcc ctgccgcggg acccctggcc ccccgcgggg atgtctgctt gagagatgcg |
| 901 | gtggctggtg gccctgaaaa ctgcctcacc agcctgaccc aggaccgggg cgggcacggc |
| 961 | gccacaggcg gctggaagct gtggtccctg tggggcgaat gcacgcggga ctgcggggga |
| 1021 | ggcctccaga cgcggacgcg cacctgcctg cccgcgccgg gcgtggaggg cggcggctgc |
| 1081 | gagggggtgc tggaggaggg tcgccagtgc aaccgcgagg cctgcggccc cgctgggcgc |
| 1141 | accagctccc ggagccagtc cctgcggtcc acagatgccc ggcggcgcga ggagctgggg |
| 1201 | gacgagctgc agcagtttgg gttcccagcc ccccagaccg gtgacccagc agccgaggag |
| 1261 | tggtccccgt ggagcgtgtg ctccagcacc tgcggcgagg gctggcagac ccgcacgcgc |
| 1321 | ttctgcgtgt cctcctccta cagcacgcag tgcagcggac ccctgcgcga gcagcggctg |
| 1381 | tgcaacaact ctgccgtgtg cccagtgcat ggtgcctggg atgagtggtc gccctggagc |
| 1441 | ctctgctcca gcacctgtgg ccgtggcttt cgggatcgca cgcgcacctg caggcccccc |
| 1501 | cagtttgggg gcaacccctg tgagggccct gagaagcaaa ccaagttctg caacattgcc |
| 1561 | ctgtgccctg gccgggcagt ggatggaaac tggaatgagt ggtcgagctg gagcgcctgc |
| 1621 | tccgccagct gctcccaggg ccgacagcag cgcacgcgtg aatgcaacgg gccttcctac |
| 1681 | gggggtgcgg agtgccaggg ccactgggtg gagacccgag actgcttcct gcagcagtgc |
| 1741 | ccagtggatg gcaagtggca ggcctgggcg tcatggggca gttgcagcgt cacgtgtggg |
| 1801 | gctggcagcc agcgacggga gcgtgtctgc tctgggccct tcttcggggg agcagcctgc |
| 1861 | cagggccccc aggatgagta ccggcagtgc ggcacccagc ggtgtcccga gccccatgag |
| 1921 | atctgtgatg aggacaactt tggtgctgtg atctggaagg agaccccagc gggagaggtg |
| 1981 | gctgctgtcc ggtgtccccg caacgccaca ggactcatcc tgcgacggtg tgagctggac |
| 2041 | gaggaaggca tcgcctactg ggagcccccc acctacatcc gctgtgtttc cattgactac |
| 2101 | agaaacatcc agatgatgac ccgggagcac ctggccaagg ctcagcgagg gctgcctggg |
| 2161 | gagggggtct cggaggtcat ccagacactg gtggagatct ctcaggacgg gaccagctac |
| 2221 | agtggggacc tgctgtccac catcgatgtc ctgaggaaca tgacagagat tttccggaga |
| 2281 | gcgtactaca gccccacccc tggggacgta cagaactttg tccagatcct tagcaacctg |
| 2341 | ttggcagagg agaatcggga caagtgggag gaggcccagc tggcgggccc caacgccaag |
| 2401 | gagctgttcc ggctggtgga ggactttgtg gacgtcatcg gcttccgcat gaaggacctg |
| 2461 | agggatgcat accaggtgac agacaacctg gttctcagca tccataagct cccagccagc |
| 2521 | ggagccactg acatcagctt ccccatgaag ggctggcggg ccacgggtga ctgggccaag |
| 2581 | gtgccagagg acagggtcac tgtgtccaag agtgtcttct ccacggggct gacagaggcc |
| 2641 | gatgaagcat ccgtgtttgt ggtgggcacc gtgctctaca ggaacctggg cagcttcctg |
| 2701 | gccctgcaga ggaacacgac cgtcctgaat tctaaggtga tctccgtgac tgtgaaaccc |
| 2761 | ccgcctcgct ccctgcgcac acccttggag atcgagtttg cccacatgta taatggcacc |
| 2821 | accaaccaga cctgtatcct gtgggatgag acggatgtac cctcctcctc cgcccccccg |
| 2881 | cagctcgggc cctggtcgtg gcgcggctgc cgcacggtgc ccctcgacgc cctccggacg |
| 2941 | cgctgcctct gtgaccggct ctccaccttc gccatcttag cccagctcag cgccgacgcg |
| 3001 | aacatggaga aggcgactct gccgtcggtg acgctcatcg tgggctgtgg cgtgtcctct |
| 3061 | ctcaccctgc tcatgctggt catcatctac gtgtccgtgt ggaggtacat tcgctcagag |
| 3121 | cgttctgtca tcctcatcaa cttctgcctg tccatcatct cctccaatgc cctcatcctc |
| 3181 | atcgggcaga cccagacccg caacaaggtg atgtgcacgc tggtggccgc cttcctgcac |
| 3241 | ttcttcttcc tgtcctcctt ctgctgggtg ctcaccgagg cctggcagtc ctacatggcc |
| 3301 | gtgacgggcc acctccggaa ccgcctcatc cgcaagcgct tcctctgcct gggctggggg |
| 3361 | ctccctgcac tggttgtggc catttctgtg ggattcacca aggccaaagg gtacagcacc |
| 3421 | atgaactact gctggctctc cctggagggg ggactgctct atgccttcgt gggacctgcc |
| 3481 | gctgccgttg tgctggtgaa catggtcatt gggatcctgg tgttcaacaa gctcgtgtcc |
| 3541 | aaagacggca tcacggacaa gaagctgaag gagcgggcag gggcctccct gtggagctcc |
| 3601 | tgcgtggtgc tgccgctgct ggcgctgacc tggatgtcgg ctgtgctcgc cgtcaccgac |
| 3661 | cgccgctccg ccctcttcca gatcctcttc gctgtcttcg actcgctgga gggcttcgtc |
| 3721 | atcgtcatgg tgcactgtat cctccgtaga gaggtccagg acgctgtgaa atgccgtgtg |
| 3781 | gttgaccggc aggaggaggg caacggggac tcagggggct ccttccagaa cggccacgcc |
| 3841 | cagctcatga ccgacttcga gaaggacgtg gatctggcct gtagatcagt gctgaacaag |
| 3901 | gacatcgcgg cctgccgcac tgccaccatc acgggcacac tgaagcggcc gtctctgccc |
| 3961 | gaggaggaga agctgaagct ggcccatgcc aaggggccgc ccaccaattt caacagcctg |
| 4021 | ccggccaacg tgtccaagct gcacctgcac ggctcacccc gctatcccgg cgggcccctg |
| 4081 | cccgacttcc ccaaccactc actgaccctc aagagggaca aggcgcccaa gtcctccttc |
| 4141 | gtcggtgacg gggacatctt caagaagctg gactcggagc tgagccgggc ccaggagaag |
| 4201 | gctctggaca cgagctacgt gatcctgccc acggccacgg ccacgctgcg gcccaagccc |
| 4261 | aaggaggagc ccaagtacag catccacatt gaccagatgc cgcagacccg cctcatccac |
| 4321 | ctcagcacgg cccccgaggc cagcctcccc gcccgcagcc cgccctcccg ccagcccccc |
| 4381 | agcggcgggc cccccgaggc accccctgcc cagcccccac cgcctccgcc cccaccgcca |
| 4441 | ccacctcccc agcagcccct gcccccaccg cccaatctgg agccggcacc ccccagcctg |
| 4501 | ggggatcccg gggagcctgc cgcccatccg ggacccagca cggggcccag caccaagaac |
| 4561 | gagaatgtcg ccaccttgtc tgtgagctcc ctggagcggc ggaagtcgcg gtatgcagaa |
| 4621 | ctggactttg agaagatcat gcacacccgg aagcggcacc aagacatgtt ccaggacctg |
| 4681 | aaccggaagc tgcagcacgc agcggagaag gacaaggagg tgctggggcc ggacagcaag |
| 4741 | ccggaaaagc agcagacgcc caacaagagg ccctgggaga gcctccggaa agcccacggg |
| 4801 | acgcccacgt gggtgaagaa ggagctggag ccgctgcagc cgtcgccgct ggagcttcgc |
| 4861 | agcgtggagt gggagaggtc gggcgccacg atcccgctgg tgggccagga catcatcgac |
| 4921 | ctccagaccg aggtctgagc gggtgggcgg cggccacgca ctgggccacg gaggagggat |
| 4981 | gctgctccgc ccgctcctgc cgcagacggg cacagacacg ctcgcgggca gcgggccagg |
| 5041 | cccgcacccc ggcctcaggg cgctcagacg gcggccaggc acagggcccg cagtgctggg |
| 5101 | accagagcca gatgcaggac aggaggcggc ccggccagcg ggcacagggc accagaggcc |
| 5161 | gaaggtgcct cagactccgc cctcctcggg ccgaggccca gcgggcagat gggcggacgg |
| 5221 | ctgtggaccg tggacaggcc cagcgcggcc agcgtcccag ggtacccgcc tgagctcctg |
| 5281 | ctgcggagga gctgcctgct tggcccggcc ggcctggcac cgttttttaa acacccccat |
| 5341 | ccctcgggaa gcagccagct ccccacacct tccagggccc taggcccctc ctagacccag |
| 5401 | gtggagggca cagccctccg accctcatgg cccccagggg caggactgag tcccctccag |
| 5461 | gaagaagcag gggggaatct attttttctc tccttttctt ttcttcaata aaaagaatta |
| 5521 | aaaacccaaa aaaaa |

### CHEF

Fusion of the signaling domain of ELMO1 to the cytoplasmic tail of two different PtdSer receptors results in ‘chimeric receptor for efferocytosis’ (CHEF). The expression of ‘chimeric receptor for efferocytosis’ (CHEF) in distinct phagocytes led to a dramatic increase in efferocytosis and dampening of inflammation in multiple in vivo disease models in mice. In mechanistic studies, protein folding in the efferocytic phagocytes was identified as a key rate-limiting step in efferocytosis that is overcome via the expression of CHEF.

The combination of TIM4 and ELMO results in TELMO. The sequence of TELMO is as follows:

TELMO (fused with GFP) nt sequence (mouse)
TIM4-capitalized letters (5′ end of molecule)
ELMO1 domain-underlined (“middle” of molecule”)
GFP-bold font (3′ end of molecule)
(SEQ ID NO: 15)
TGTCCAAGGGGCTTCTCCTCCTCTGGCTGGTGACGGAGCTCTGGTGGCTTTATCT
GACACCAGCTGCCTCAGAGGATACAATAATAGGGTTTTTGGGCCAGCCGGTGAC
TTTGCCTTGTCATTACCTCTCGTGGTCCCAGAGCCGCAACAGTATGTGCTGGGGC
AAAGGTTCATGTCCCAATTCCAAGTGCAATGCAGAGCTTCTCCGTACAGATGGAA
CAAGAATCATCTCCAGGAAGTCAACAAAATATACACTTTTGGGGAAGGTCCAGT
TTGGTGAAGTGTCCTTGACCATCTCAAACACCAATCGAGGTGACAGTGGGGTGTA
CTGCTGCCGTATAGAGGTGCCTGGCTGGTTCAATGATGTCAAGAAGAATGTGCGC
TTGGAGCTGAGGAGAGCCACAACAACCAAAAAACCAACAACAACCACCCGGCC
AACCACCACCCCTTATGTGACCACCACCACCCCAGAGCTGCTTCCAACAACAGTC
ATGACCACATCTGTTCTCCCAACCACCACACCACCCCAGACACTAGCCACCACTG
CCTTCAGTACAGCAGTGACCACGTGCCCCTCAACAACACCTGGCTCCTTCTCACA
AGAAACCACAAAAGGGTCCGCCTTCACTACAGAATCAGAAACTCTGCCTGCATC
CAATCACTCTCAAAGAAGCATGATGACCATATCTACAGACATAGCCGTACTCAG
GCCCACAGGCTCTAACCCTGGGATTCTCCCATCCACTTCACAGCTGACGACACAG
AAAACAACATTAACAACAAGTGAGTCTTTGCAGAAGACAACTAAATCACATCAG
ATCAACAGCAGACAGACCATCTTGATCATTGCCTGCTGTGTGGGATTTGTGCTAA
TGGTGTTATTGTTTCTGGCGTTTCTCCTTCGAGGGAAAGTCACAGGAGCCAACTG
TTTGCAGAGACACAAGAGGCCAGACAACACTGAAGATAGTGACAGCGTCcatcatca
ccatcaccattcccgggttccgattttggaactaaaggagaagatccagccagaaatcttagagctgattaaacagcagcgcctgaacc
gccttgtggaagggacctgctttaggaaactcaatgctcgccggagacaagacaagttttggtattgtcggctttcaccaaatcacaag
gtcttacattatggcgacttggaagagagcccccaaggagaagtgccccacgattccctgcaggacaaattgccggtggcagatatca
aagcagtggtgacgggaaaggactgccctcatatgaaagagaaaggtgcccttaaacaaaacaaggaggtgcttgaacttgctttctc
catcttatacgactcaaattgccaactgaacttcattgctcctgataagcatgagtactgcatctggacagatgggctgaatgcactgcttg
ggaaggacatgatgagtgacctgacacgcaatgacctggacaccctgctgagcatggagatcaagcttcgcctgctggacctggaaa
acatccagatcccggatgcacctccgcctatccccaaggaacctagcaactatgactttgtctatgactgtaacacccgggatccaccg
gtcgccaccatggtgagcaagggcgaggagctgttcaccggggtggtgcccatcctggtcgagctggacggcgacgtaaacg
gccacaagttcagcgtgtccggcgagggcgagggcgatgccacctacggcaagctgaccctgaagttcatctgcaccaccgg
caagctgcccgtgccctggcccaccctcgtgaccaccctgacctacggcgtgcagtgcttcagccgctaccccgaccacatga
agcagcacgacttcttcaagtccgccatgcccgaaggctacgtccaggagcgcaccatcttcttcaaggacgacggcaactac
aagacccgcgccgaggtgaagttcgagggcgacaccctggtgaaccgcatcgagctgaagggcatcgacttcaaggaggac
ggcaacatcctggggcacaagctggagtacaactacaacagccacaacgtctatatcatggccgacaagcagaagaacggc
atcaaggtgaacttcaagatccgccacaacatcgaggacggcagcgtgcagctcgccgaccactaccagcagaacaccccc
atcggcgacggccccgtgctgctgcccgacaaccactacctgagcacccagtccgccctgagcaaagaccccaacgagaag
cgcgatcacatggtcctgctggagttcgtgaccgccgccgggatcactctcggcatggacgagctgtacaagtaa
TELMO protein (fused with GFP) sequence (mouse)
(SEQ ID NO: 16)
MSKGLLLLWLVTELWWLYLTPAASEDTIIGFLGQPVTLPCHYLSWSQSRNSMCWGK
GSCPNSKCNAELLRTDGTRIISRKSTKYTLLGKVQFGEVSLTISNTNRGDSGVYCCRIE
VPGWFNDVKKNVRLELRRATTTKKPTTTTRPTTTPYVTTTTPELLPTTVMTTSVLPTT
TPPQTLATTAFSTAVTTCPSTTPGSFSQETTKGSAFTTESETLPASNHSQRSMMTISTDI
AVLRPTGSNPGILPSTSQLTTQKTTLTTSESLQKTTKSHQINSRQTILIIACCVGFVLMV
LLFLAFLLRGKVTGANCLQRHKRPDNTEDSDSVHHHHHHSRVPILELKEKIQPEILELI
KQQRLNRLVEGTCFRKLNARRRQDKFWYCRLSPNHKVLHYGDLEESPQGEVPHDSL
QDKLPVADIKAVVTGKDCPHMKEKGALKQNKEVLELAFSILYDSNCQLNFIAPDKH
EYCIWTDGLNALLGKDMMSDLTRNDLDTLLSMEIKLRLLDLENIQIPDAPPPIPKEPS
NYDFVYDCNTRDPPVATMVSKGEELFTGVVPILVELDGDVNGHKFSVSGEGEGDAT
YGKLTLKFICTTGKLPVPWPTLVTTLTYGVQCFSRYPDHMKQHDFFKSAMPEGYVQ
ERTIFFKDDGNYKTRAEVKFEGDTLVNRIELKGIDFKEDGNILGHKLEYNYNSHNVYI
MADKQKNGIKVNFKIRHNIEDGSVQLADHYQQNTPIGDGPVLLPDNHYLSTQSALSK
DPNEKRDHMVLLEFVTAAGITLGMDELYK*

The combination of BAI1 and ELMO results in BELMO. The sequence of BELMO is as follows:

BELMO (fused with GFP) nt sequence (mouse)
BAI1-capitalized letters (5′ end of molecule)
ELMO1 domain-underlined (“middle” of molecule”)
GFP-bold font (3′ end of molecule)
(SEQ ID NO: 17)
ATGAGGGGCCAGGCCGCCGCCCCGGGCCCCGTCTGGATCCTCGCCCCGCTGCTAC
TGCTGCTGCTGCTGCTGGGACGCCGCGCGCGGGCGGCCGCCGGAGCAGACGCGG
GGCCCGGGCCCGAGCCGTGCGCCACGCTGGTGCAGGGAAAGTTCTTCGGCTACT
TCTCCGCGGCCGCCGTGTTCCCGGCCAACGCCTCGCGCTGCTCCTGGACGCTACG
CAACCCGGACCCGCGGCGCTACACTCTCTACATGAAGGTGGCCAAGGCGCCCGT
GCCCTGCAGCGGCCCCGGCCGCGTGCGCACCTACCAGTTCGACTCCTTCCTCGAG
TCCACGCGCACCTACCTGGGCGTGGAGAGCTTCGACGAGGTGCTGCGGCTCTGC
GACCCCTCCGCACCCCTGGCCTTCCTGCAGGCCAGCAAGCAGTTCCTGCAGATGC
GGCGCCAGCAGCCGCCCCAGCACGACGGGCTCCGGCCCCGGGCCGGGCCGCCGG
GCCCCACCGACGACTTCTCCGTGGAGTACCTGGTGGTGGGGAACCGCAACCCCA
GCCGTGCCGCCTGCCAGATGCTGTGCCGCTGGCTGGACGCGTGTCTGGCCGGTAG
TCGCAGCTCGCACCCCTGCGGGATCATGCAGACCCCCTGCGCCTGCCTGGGCGGC
GAGGCGGGCGGCCCTGCCGCGGGACCCCTGGCCCCCCGCGGGGATGTCTGCTTG
AGAGATGCGGTGGCTGGTGGCCCTGAAAACTGCCTCACCAGCCTGACCCAGGAC
CGGGGCGGGCACGGCGCCACAGGCGGCTGGAAGCTGTGGTCCCTGTGGGGCGAA
TGCACGCGGGACTGCGGGGGAGGCCTCCAGACGCGGACGCGCACCTGCCTGCCC
GCGCCGGGCGTGGAGGGCGGCGGCTGCGAGGGGGTGCTGGAGGAGGGTCGCCA
GTGCAACCGCGAGGCCTGCGGCCCCGCTGGGCGCACCAGCTCCCGGAGCCAGTC
CCTGCGGTCCACAGATGCCCGGCGGCGCGAGGAGCTGGGGGACGAGCTGCAGCA
GTTTGGGTTCCCAGCCCCCCAGACCGGTGACCCAGCAGCCGAGGAGTGGTCCCC
GTGGAGCGTGTGCTCCAGCACCTGCGGCGAGGGCTGGCAGACCCGCACGCGCTT
CTGCGTGTCCTCCTCCTACAGCACGCAGTGCAGCGGACCCCTGCGCGAGCAGCG
GCTGTGCAACAACTCTGCCGTGTGCCCAGTGCATGGTGCCTGGGATGAGTGGTCG
CCCTGGAGCCTCTGCTCCAGCACCTGTGGCCGTGGCTTTCGGGATCGCACGCGCA
CCTGCAGGCCCCCCCAGTTTGGGGGCAACCCCTGTGAGGGCCCTGAGAAGCAAA
CCAAGTTCTGCAACATTGCCCTGTGCCCTGGCCGGGCAGTGGATGGAAACTGGA
ATGAGTGGTCGAGCTGGAGCGCCTGCTCCGCCAGCTGCTCCCAGGGCCGACAGC
AGCGCACGCGTGAATGCAACGGGCCTTCCTACGGGGGTGCGGAGTGCCAGGGCC
ACTGGGTGGAGACCCGAGACTGCTTCCTGCAGCAGTGCCCAGTGGATGGCAAGT
GGCAGGCCTGGGCGTCATGGGGCAGTTGCAGCGTCACGTGTGGGGCTGGCAGCC
AGCGACGGGAGCGTGTCTGCTCTGGGCCCTTCTTCGGGGGAGCAGCCTGCCAGG
GCCCCCAGGATGAGTACCGGCAGTGCGGCACCCAGCGGTGTCCCGAGCCCCATG
AGATCTGTGATGAGGACAACTTTGGTGCTGTGATCTGGAAGGAGACCCCAGCGG
GAGAGGTGGCTGCTGTCCGGTGTCCCCGCAACGCCACAGGACTCATCCTGCGAC
GGTGTGAGCTGGACGAGGAAGGCATCGCCTACTGGGAGCCCCCCACCTACATCC
GCTGTGTTTCCATTGACTACAGAAACATCCAGATGATGACCCGGGAGCACCTGGC
CAAGGCTCAGCGAGGGCTGCCTGGGGAGGGGGTCTCGGAGGTCATCCAGACACT
GGTGGAGATCTCTCAGGACGGGACCAGCTACAGTGGGGACCTGCTGTCCACCAT
CGATGTCCTGAGGAACATGACAGAGATTTTCCGGAGAGCGTACTACAGCCCCAC
CCCTGGGGACGTACAGAACTTTGTCCAGATCCTTAGCAACCTGTTGGCAGAGGA
GAATCGGGACAAGTGGGAGGAGGCCCAGCTGGCGGGCCCCAACGCCAAGGAGC
TGTTCCGGCTGGTGGAGGACTTTGTGGACGTCATCGGCTTCCGCATGAAGGACCT
GAGGGATGCATACCAGGTGACAGACAACCTGGTTCTCAGCATCCATAAGCTCCC
AGCCAGCGGAGCCACTGACATCAGCTTCCCCATGAAGGGCTGGCGGGCCACGGG
TGACTGGGCCAAGGTGCCAGAGGACAGGGTCACTGTGTCCAAGAGTGTCTTCTC
CACGGGGCTGACAGAGGCCGATGAAGCATCCGTGTTTGTGGTGGGCACCGTGCT
CTACAGGAACCTGGGCAGCTTCCTGGCCCTGCAGAGGAACACGACCGTCCTGAA
TTCTAAGGTGATCTCCGTGACTGTGAAACCCCCGCCTCGCTCCCTGCGCACACCC
TTGGAGATCGAGTTTGCCCACATGTATAATGGCACCACCAACCAGACCTGTATCC
TGTGGGATGAGACGGATGTACCCTCCTCCTCCGCCCCCCCGCAGCTCGGGCCCTG
GTCGTGGCGCGGCTGCCGCACGGTGCCCCTCGACGCCCTCCGGACGCGCTGCCTC
TGTGACCGGCTCTCCACCTTCGCCATCTTAGCCCAGCTCAGCGCCGACGCGAACA
TGGAGAAGGCGACTCTGCCGTCGGTGACGCTCATCGTGGGCTGTGGCGTGTCCTC
TCTCACCCTGCTCATGCTGGTCATCATCTACGTGTCCGTGTGGAGGTACATTCGCT
CAGAGCGTTCTGTCATCCTCATCAACTTCTGCCTGTCCATCATCTCCTCCAATGCC
CTCATCCTCATCGGGCAGACCCAGACCCGCAACAAGGTGATGTGCACGCTGGTG
GCCGCCTTCCTGCACTTCTTCTTCCTGTCCTCCTTCTGCTGGGTGCTCACCGAGGC
CTGGCAGTCCTACATGGCCGTGACGGGCCACCTCCGGAACCGCCTCATCCGCAA
GCGCTTCCTCTGCCTGGGCTGGGGGCTCCCTGCACTGGTTGTGGCCATTTCTGTG
GGATTCACCAAGGCCAAAGGGTACAGCACCATGAACTACTGCTGGCTCTCCCTG
GAGGGGGGACTGCTCTATGCCTTCGTGGGACCTGCCGCTGCCGTTGTGCTGGTGA
ACATGGTCATTGGGATCCTGGTGTTCAACAAGCTCGTGTCCAAAGACGGCATCAC
GGACAAGAAGCTGAAGGAGCGGGCAGGGGCCTCCCTGTGGAGCTCCTGCGTGGT
GCTGCCGCTGCTGGCGCTGACCTGGATGTCGGCTGTGCTCGCCGTCACCGACCGC
CGCTCCGCCCTCTTCCAGATCCTCTTCGCTGTCTTCGACTCGCTGGAGGGCTTCGT
CATCGTCATGGTGCACTGTATCCTCCGTAGAGAGGTCCAGGACGCTGTGAAATGC
CGTGTGGTTGACCGGCAGGAGGAGGGCAACGGGGACTCAGGGGGCTCCTTCCAG
AACGGCCACGCCCAGCTCATGACCGACTTCGAGAAGGACGTGcatcatcaccatcaccatgtc
gagccgattttggaactaaaggagaagatccagccagaaatcttagagctgattaaacagcagcgcctgaaccgccttgtggaaggg
acctgctttaggaaactcaatgctcgccggagacaagacaagttttggtattgtcggctttcaccaaatcacaaggtcttacattatggcg
acttggaagagagcccccaaggagaagtgccccacgattccctgcaggacaaattgccggtggcagatatcaaagcagtggtgacg
ggaaaggactgccctcatatgaaagagaaaggtgcccttaaacaaaacaaggaggtgcttgaacttgctttctccattttatacgactca
aattgccaactgaacttcattgctcctgataagcatgagtactgcatctggacagatgggctgaatgcactgcttgggaaggacatgatg
agtgacctgacacgcaatgacctggacaccctgctgagcatggagatcaagcttcgcctgctggacctggaaaacatccagatcccg
gatgcacctccgcctatccccaaggaacctagcaactatgactttgtctatgactgtaacgtcgacggaggggtaccgcgggcccgg
gatccaccggtcgccaccatggtgagcaagggcgaggagctgttcaccggggtggtgcccatcctggtcgagctggacggcg
acgtaaacggccacaagttcagcgtgtccggcgagggcgagggcgatgccacctacggcaagctgaccctgaagttcatctg
caccaccggcaagctgcccgtgccctggcccaccctcgtgaccaccctgacctacggcgtgcagtgcttcagccgctaccccg
accacatgaagcagcacgacttcttcaagtccgccatgcccgaaggctacgtccaggagcgcaccatcttcttcaaggacgac
ggcaactacaagacccgcgccgaggtgaagttcgagggcgacaccctggtgaaccgcatcgagctgaagggcatcgacttc
aaggaggacggcaacatcctggggcacaagctggagtacaactacaacagccacaacgtctatatcatggccgacaagcag
aagaacggcatcaaggtgaacttcaagatccgccacaacatcgaggacggcagcgtgcagctcgccgaccactaccagcag
aacacccccatcggcgacggccccgtgctgctgcccgacaaccactacctgagcacccagtccgccctgagcaaagacccca
acgagaagcgcgatcacatggtcctgctggagttcgtgaccgccgccgggatcactctcggcatggacgagctgtacaagta
a
BELMO protein (fused with GFP) sequence (mouse)
(SEQ ID NO: 18)
MRGQAAAPGPVWILAPLLLLLLLLGRRARAAAGADAGPGPEPCATLVQGKFFGYFS
AAAVFPANASRCSWTLRNPDPRRYTLYMKVAKAPVPCSGPGRVRTYQFDSFLESTR
TYLGVESFDEVLRLCDPSAPLAFLQASKQFLQMRRQQPPQHDGLRPRAGPPGPTDDF
SVEYLVVGNRNPSRAACQMLCRWLDACLAGSRSSHPCGIMQTPCACLGGEAGGPA
AGPLAPRGDVCLRDAVAGGPENCLTSLTQDRGGHGATGGWKLWSLWGECTRDCG
GGLQTRTRTCLPAPGVEGGGCEGVLEEGRQCNREACGPAGRTSSRSQSLRSTDARRR
EELGDELQQFGFPAPQTGDPAAEEWSPWSVCSSTCGEGWQTRTRFCVSSSYSTQCSG
PLREQRLCNNSAVCPVHGAWDEWSPWSLCSSTCGRGFRDRTRTCRPPQFGGNPCEG
PEKQTKFCNIALCPGRAVDGNWNEWSSWSACSASCSQGRQQRTRECNGPSYGGAEC
QGHWVETRDCFLQQCPVDGKWQAWASWGSCSVTCGAGSQRRERVCSGPFFGGAA
CQGPQDEYRQCGTQRCPEPHEICDEDNFGAVIWKETPAGEVAAVRCPRNATGLILRR
CELDEEGIAYWEPPTYIRCVSIDYRNIQMMTREHLAKAQRGLPGEGVSEVIQTLVEIS
QDGTSYSGDLLSTIDVLRNMTEIFRRAYYSPTPGDVQNFVQILSNLLAEENRDKWEE
AQLAGPNAKELFRLVEDFVDVIGFRMKDLRDAYQVTDNLVLSIHKLPASGATDISFP
MKGWRATGDWAKVPEDRVTVSKSVFSTGLTEADEASVFVVGTVLYRNLGSFLALQ
RNTTVLNSKVISVTVKPPPRSLRTPLEIEFAHMYNGTTNQTCILWDETDVPSSSAPPQL
GPWSWRGCRTVPLDALRTRCLCDRLSTFAILAQLSADANMEKATLPSVTLIVGCGVS
SLTLLMLVIIYVSVWRYIRSERSVILINFCLSIISSNALILIGQTQTRNKVMCTLVAAFL
HFFFLSSFCWVLTEAWQSYMAVTGHLRNRLIRKRFLCLGWGLPALVVAISVGFTKA
KGYSTMNYCWLSLEGGLLYAFVGPAAAVVLVNMVIGILVFNKLVSKDGITDKKLKE
RAGASLWSSCVVLPLLALTWMSAVLAVTDRRSALFQILFAVFDSLEGFVIVMVHCIL
RREVQDAVKCRVVDRQEEGNGDSGGSFQNGHAQLMTDFEKDVHHHHHHVEPILEL
KEKIQPEILELIKQQRLNRLVEGTCFRKLNARRRQDKFWYCRLSPNHKVLHYGDLEE
SPQGEVPHDSLQDKLPVADIKAVVTGKDCPHMKEKGALKQNKEVLELAFSILYDSN
CQLNFIAPDKHEYCIWTDGLNALLGKDMMSDLTRNDLDTLLSMEIKLRLLDLENIQIP
DAPPPIPKEPSNYDFVYDCNVDGGVPRARDPPVATMVSKGEELFTGVVPILVELDGD
VNGHKFSVSGEGEGDATYGKLTLKFICTTGKLPVPWPTLVTTLTYGVQCFSRYPDH
MKQHDFFKSAMPEGYVQERTIFFKDDGNYKTRAEVKFEGDTLVNRIELKGIDFKED
GNILGHKLEYNYNSHNVYIMADKQKNGIKVNFKIRHNIEDGSVQLADHYQQNTPIG
DGPVLLPDNHYLSTQSALSKDPNEKRDHMVLLEFVTAAGITLGMDELYK*

### Administration/Expression Systems

In some embodiments, protein, RNA and/or DNA are administered to a subject.

The administration can be local to an organ or tissue by, for example, injection or with the use of a catheter or during surgery. The administration can also be systemic, such as by an injection.

The nucleic acids provided herein (RNA or DNA) can be linear (similar to RNA used in the COVID-19 vaccines). RNA can be administered similar to RNA in COVID-19 vaccines. Nucleic acids provide herein can part of an expression vector, which can be a viral vector, to express the fusion protein in one or more cells. An adenoviral expression system can also be used. Lipid particles or viral particles can be used as delivery vehicles as well. Delivery vehicles can be associated with one or more targeting molecules so as to target specific cells.

The protein, RNA and/or DNA can be present in a pharmaceutically acceptable carrier. As used herein, the term “pharmaceutically-acceptable carrier” means a chemical composition with which an appropriate compound or derivative can be combined and which, following the combination, can be used to administer the appropriate compound to a subject.

Pharmaceutically acceptable carriers include physiologically tolerable or acceptable diluents, excipients, solvents or adjuvants. The compositions are preferably sterile and nonpyrogenic. Examples of suitable carriers include, but are not limited to, water, normal saline, dextrose, mannitol, lactose or other sugars, lecithin, albumin, sodium glutamate, cysteine hydrochloride, ethanol, polyols (propylene glycol, polyethylene glycol, glycerol, and the like), vegetable oils (such as olive oil), injectable organic esters such as ethyl oleate, ethoxylated isosteraryl alcohols, polyoxyethylene sorbitol and sorbitan esters, microcrystalline cellulose, aluminum methahydroxide, bentonite, kaolin, agar-agar and tragacanth, lipids/liposomes, lipid nanoparticles or mixtures of these substances, and the like.

The pharmaceutical compositions may also contain minor amounts of nontoxic auxiliary pharmaceutical substances or excipients and/or additives, such as wetting agents, emulsifying agents, pH buffering agents, antibacterial and antifungal agents (such as parabens, chlorobutanol, phenol, sorbic acid, and the like). Suitable additives include, but are not limited to, physiologically biocompatible buffers (e.g., tromethamine hydrochloride), additions (e.g., 0.01 to 10 mole percent) of chelants (such as, for example, DTPA or DTPA-bisamide) or calcium chelate complexes (as for example calcium DTPA or CaNaDTPA-bisamide), or, optionally, additions (e.g. 1 to 50 mole percent) of calcium or sodium salts (for example, calcium chloride, calcium ascorbate, calcium gluconate or calcium lactate). If desired, absorption enhancing or delaying agents (such as liposomes, aluminum monostearate, or gelatin) may be used. The compositions can be prepared in conventional forms, either as liquid solutions or suspensions, solid forms suitable for solution or suspension in liquid prior to injection, or as emulsions. Pharmaceutical compositions according to the present invention can be prepared in a manner fully within the skill of the art.

Nucleic acids coding for the fusion proteins provided herein (or one more domains of the fusion protein) can be incorporated into and expressed from one or more expression cassettes or expression vectors.

A “vector” is a composition of matter that can be used to deliver a nucleic acid of interest to the interior of a cell. Nucleic acids encoding a fusion protein can be introduced into a cell via a single vector or via multiple separate vectors to allow expression thereof in host cells.

Vectors typically include control elements operably linked to the fusion protein sequences, which allow for expression in vivo in cells. For example, the segment encoding the fusion protein can be operably linked to a promoter to allow expression thereof.

Numerous vectors are available including, but not limited to, linear polynucleotides, polynucleotides associated with ionic or amphiphilic compounds, plasmids, and viruses. Thus, the term “vector” includes an autonomously replicating plasmid. An expression construct can be replicated in a living cell, or it can be made synthetically. For purposes of this application, the terms “expression construct,” “expression vector,” and “vector,” are used interchangeably to demonstrate the application of the invention in a general, illustrative sense, and are not intended to limit the invention.

In certain embodiments, the nucleic acid comprising one or more wild type or modified sequences is under transcriptional control of a promoter. A “promoter” refers to a DNA sequence recognized by the synthetic machinery of the cell, or introduced synthetic machinery, required to initiate the specific transcription of a gene. The term promoter will be used here to refer to a group of transcriptional control modules that are clustered around the initiation site for RNA polymerase. Such promoters can be obtained from commercially available plasmids, using techniques available in the art. See, e.g., Sambrook et al., supra. Enhancer elements may be used in association with the promoter to increase expression levels of the constructs.

Expression vectors for expressing one or more products or nucleic acids can include a promoter “operably linked” to a nucleic acid segment encoding the product of interest. The phrase “operably linked” or “under transcriptional control” as used herein means that the promoter is in the correct location and orientation in relation to a polynucleotide to control the initiation of transcription by RNA polymerase and expression of the product.

Typically, transcription terminator/polyadenylation signals will also be present in the expression construct.

In order to effect expression of the fusion protein one or more expression constructs can be delivered into a cell. This delivery may be accomplished in vitro, as in laboratory procedures for transforming host cell lines, or in vivo.

Delivery of constructs encoding the fusion protein to a cell can be accomplished with or without vectors.

In yet another embodiment, the expression construct may simply consist of naked recombinant DNA or plasmids. This is particularly applicable for transfer in vitro but it may be applied to in vivo use as well.

In a further embodiment, the expression construct may be delivered using liposomes. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh & Bachhawat (1991) Liver Diseases, Targeted Diagnosis and Therapy Using Specific Receptors and Ligands, Wu et al. (Eds.), Marcel Dekker, NY, 87-104). Also contemplated is the use of lipofectamine-DNA complexes.

In some cases, a construct encoding a fusion protein may be contacted with host cells in combination with a cationic lipid. Examples of cationic lipids include, but are not limited to, lipofectin, DOTMA, DOPE, and DOTAP. The publication of WO/0071096, which is specifically incorporated by reference, describes different formulations, such as a DOTAP:cholesterol or cholesterol derivative formulation that can effectively be used for gene therapy. Other disclosures also discuss different lipid or liposomal formulations including nanoparticles and methods of administration; these include, but are not limited to, U.S. Patent Publication 20030203865, 20020150626, 20030032615, and 20040048787, which are specifically incorporated by reference to the extent they disclose formulations and other related aspects of delivery of nucleic acids. Methods used for forming particles are also disclosed in U.S. Pat. Nos. 5,844,107, 5,877,302, 6,008,336, 6,077,835, 5,972,901, 6,200,801, and 5,972,900, which are incorporated by reference for those aspects.

### Example I

### Introduction

The clearance of apoptotic cells by phagocytes, a process termed efferocytosis, is essential for maintaining tissue homeostasis (Boada-Romero et al., 2020; Doran et al., 2020; Elliott and Ravichandran, 2016; Gregory and Pound, 2010). 200-300 billion cells die by apoptosis each day and are cleared via efferocytosis without an inflammatory consequence. This efferocytic process is carried out by professional phagocytes, such as macrophages, as well as non-professional phagocytes, including epithelial cells, fibroblasts, and endothelial cells (Boada-Romero et al., 2020; Doran et al., 2020; Elliott and Ravichandran, 2016; Gregory and Pound, 2010; Han et al., 2016; Juncadella et al., 2013; Lemke, 2019; Monks et al., 2008; Morioka et al., 2019; Shankman et al., 2021). As apoptosis can increase locally under pathological conditions, including tissue injury, infection, neurodegenerative diseases, and autoimmune diseases, apoptotic cells can accumulate; in such cases, uncleared apoptotic cells can advance to secondary necrosis and in turn, exacerbate inflammation and pathology. Thus, defective efferocytosis is considered a contributing factor to a growing list of human diseases, including atherosclerosis, systemic lupus erythematosus and atherosclerosis, and ulcerative colitis (Boada-Romero et al., 2020; Doran et al., 2020; Elliott and Ravichandran, 2016; Gregory and Pound, 2010; Henson, 2017; Henson and Hume, 2006; Lemke, 2019; Morioka et al., 2019; Nagata, 2018; Rothlin et al., 2021).

In terms of recognizing and removing apoptotic cells, the exposure of phosphatidylserine (PtdSer) on apoptotic cells plays a central role. After apoptosis induction, PtdSer is translocated from the inner to the outer leaflet of the plasma membrane, and several phagocytic receptors on phagocytes have been identified to engage PtdSer through direct or indirect interactions (Boada-Romero et al., 2020; Doran et al., 2020; Gregory and Pound, 2010; Morioka et al., 2019; Nagata, 2018; Rothlin et al., 2021). Integrins (ανP3 and αvβ5) and the Tyro3/Axl/Mer (TAM) family of tyrosine kinase receptors recognize apoptotic cells indirectly through association with secreted PtdSer-binding proteins MFG-E8 and Gas6/Protein S, respectively (Boada-Romero et al., 2020; Doran et al., 2020; Elliott and Ravichandran, 2016; Gregory and Pound, 2010; Morioka et al., 2019; Nagata, 2018; Rothlin et al., 2021). Among the direct PtdSer recognition receptors, TIM4 binds PtdSer directly, but due to the lack of an intracellular signaling domain, it acts as a tethering receptor (Nagata, 2018; Park et al., 2009). BAI1 is another direct PtdSer binding receptor and, via its cytoplasmic tail domain, interacts with the engulfment promoting cytoplasmic protein, ELMO. In turn, ELMO associates with Dock180, and the ELMO/Dock complex functions as a guanine nucleotide exchange factor for the small GTPase Rac1 to promote cytoskeletal reorganization during the engulfment of apoptotic cells (Brugnera et al., 2002; Gumienny et al., 2001; Park et al., 2007). The Dock/ELMO/Rac module is highly conserved in evolution and regulates efferocytosis in C. elegans, drosophila, zebrafish, and mammals (Elliott et al., 2010; Epting et al., 2010; Geisbrecht et al., 2008; Gumienny et al., 2001). In support of this conserved function, transgenic expression of human ELMO1 could rescue efferocytosis and cell motility defects in CED-12/ELMO-1 mutant C. elegans (Gumienny et al., 2001; Tosello-Trampont et al., 2007). Extensive biochemical and recent structural studies on Dock/ELMO/Rac complex have revealed the distinct domains of ELMO1 responsible for the interaction with Dock and Rac proteins and in turn, the functional regions required for ELMO-mediated signaling during efferocytosis (Chang et al., 2020; deBakker et al., 2004; Elliott et al., 2010; Gumienny et al., 2001; Lu et al., 2004; Park et al., 2007; Tosello-Trampont et al., 2007).

During efferocytosis, phagocytes simultaneously coordinate corpse uptake, process the ingested materials, and secrete anti-inflammatory mediators while maintaining their cellular homeostasis/metabolism (Boada-Romero et al., 2020; Doran et al., 2020; Morioka et al., 2019). While elegant works from a number of laboratories are beginning to define the molecules and pathways that critically affect the different steps of efferocytosis, the complexity of the process that involves a phagocyte taking up another cell nearly its own size and often eating successive corpses, make our knowledge of the efferocytic process and the molecular mechanisms are far from complete. While knockouts and other genetic approaches (across the species spectrum) have identified key specific receptors and molecules involved in efferocytosis, how to ‘manipulate’ these molecules to improve efferocytosis could point to potential approaches to limit tissue injury and preserve the function.

In this work, a strategy was designed to fuse the signaling domain of ELMO1 to the cytoplasmic tail of two different PtdSer receptors. The expression of ‘chimeric receptor for efferocytosis’ (CHEF) in distinct phagocytes led to a dramatic increase in efferocytosis and dampening of inflammation in multiple in vivo disease models in mice. In mechanistic studies, we also identify protein folding in the efferocytic phagocytes as a key rate-limiting step in efferocytosis that is overcome via the expression of CHEF.

### Results

### Engineering Chimeric Efferocytosis Receptors for Boosting Efferocytosis

While attempting to define the relevance of specific efferocytic receptors, multiple studies have overexpressed engulfment receptors in cell types that either already express or lack a particular receptor (Fourgeaud et al., 2016; Lee et al., 2016; Miyanishi et al., 2007; Yanagihashi et al., 2017; Zhu et al., 2015). Such efferocytic receptor expression often enhances basal efferocytosis. However, an inherent limitation is that the receptor still has to recruit cytoplasmic signaling intermediates, which may limit their efficacy. Further, as a given cell type may or may not express the appropriate downstream signaling intermediates for a specific efferocytic receptor, the effects on efferocytosis are less predictable across cell types. A new approach is described herein to overcome these issues. In this approach, efferocytic receptors were directly fused to a specific signaling intermediate. For the intracellular signaling intermediate, a domain of ELMO1 encompassing the C-terminal 195 amino acids was chosen for two reasons: first, this region can directly bind Dock180, this ‘partial’ complex can efficiently mimic the Rac activation and actin rearrangement activities of the native ELMO/Dock180 complex (Grimsley et al., 2004; Lu et al., 2004; Lu et al., 2005; Patel et al., 2011); second, the different ELMO and Dock homologs are expressed in essentially all cell types, and thus it was envisioned that this could signal in different phagocytic cell types and even compensate across evolution (Grimsley et al., 2004; Gumienny et al., 2001; Tosello-Trampont et al., 2007). AA 533-727 region of ELMO1 was directed fused to the efferocytic receptor BAI1. BAI1 was chosen for two reasons: first, BAI1 naturally binds ELMO1, and the BAI1-ELMO1-Dock180-Rac module can promote efferocytosis (FIG. 1A); second, BAI1 directly binds PtdSer, the most evolutionarily conserved ligand on apoptotic cells (Park et al., 2007). After deleting the natural ELMO1 binding site within the cytoplasmic tail of BAI1, this truncated BAI1 (aa 1-1180) was directly fused to the 533-727 region of ELMO1. This chimeric efferocytosis receptor is denoted as ‘BELMO’ (FIGS. 1A and S1A).

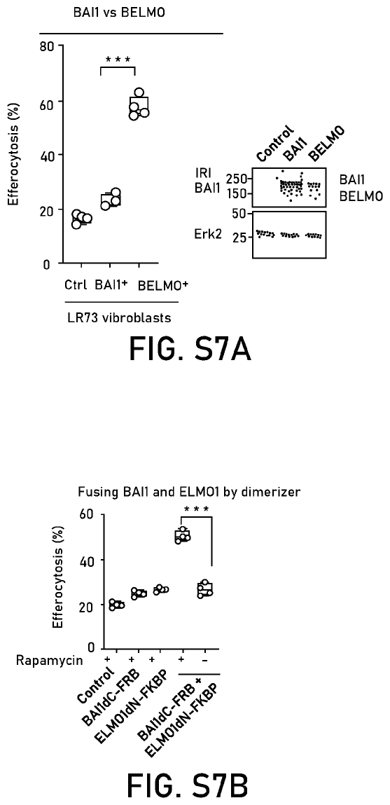

After confirming that BELMO is expressed on the cell surface (FIG. 1), efferocytosis assays were performed. BELMO expression provided a striking 5-fold increase in phagocytosis of apoptotic cells (FIG. 1C). Time-lapse imaging and flow cytometric analyses demonstrated that BELMO expressing cells take up more corpses per phagocyte compared to control vector expressing phagocytes (FIG. 1D and S1B). The enhanced efferocytosis due to BELMO overexpression was greater than native BAI1 overexpression or ELMO1 overexpression. Also, the effect of BELMO was simply due to membrane targeting of a critical signaling region of ELMO, as CAAX signal peptide-mediated membrane-targeted ELMO1 did not promote efferocytosis; further, co-expression of native BAI1 with membrane-targeted ELMO1 also did not achieve the dramatic increase in efferocytosis seen with BELMO (FIG. SIC). Thus, the chimeric fusion of BAI1 with ELMO provided a significant boost in efferocytosis. Next, additional features of BELMO-mediated efferocytosis were tested: first, while BELMO strongly promoted apoptotic cell uptake, it did not trigger live cell uptake (FIG. 1E); second, masking PtdSer on apoptotic cells with annexin V inhibited BELMO-mediated uptake, demonstrating PtdSer-dependence (FIG. 1F); third, inhibiting RacI activity abolished BELMO-driven enhanced phagocytosis (FIG. 1G); fourth, preventing actin polymerization by Cytochalasin D suppressed BELMO-dependent efferocytosis (FIG. 1H); fifth, BELMO 6M, a variant of BELMO where signaling residues were mutated within the ELMO1 sequence (FIG. 11) (Grimsley et al., 2004), failed to enhance efferocytosis (note: BELMO 6M was expressed on the cell surface and did not function as a dominant-negative either) (FIG. 1I and S1D); and sixth, BELMO interacted with native Dock180 in a ligand-dependent manner (FIGS. S1E), consistent with the requirement for functional Rac1 for BELMO mediated enhanced efferocytosis (FIG. 1G). Furthermore, when corpse acidification after BELMO-mediated efferocytosis was analyzed (by feeding CellTraceViolet-labeled apoptotic cells and tracking quenching), BELMO⁺ phagocytes efficiently acidified the ingested apoptotic cells (FIG. S1F). These data demonstrate that chimeric efferocytic receptor BELMO is a bonafide and highly efficient engulfment receptor for the clearance of apoptotic cells in vitro.

### Generating Conditional Transgenic Animal Models for Boosting Efferocytosis

To test whether BELMO is functional in vivo, two approaches were taken. First, transgenic zebrafish expressing BELMO were engineered. BELMO was expressed in glia (marked with eGFP using the slc1a3b promoter) via the doxycycline-inducible Tet-On system harboring biTRE (FIG. 2A, see Methods for detail); this allows one to express BELMO and a concurrent nuclear-targeted nls-mCherry to identify BELMO expressing glia (FIG. 2A and S2A). The transgenic DNA constructs were injected into the 1-cell embryo stage, and BELMO expression was induced at day 3 post-fertilization by doxycycline treatment for 24 hours. To test the function of BELMO expressing cells, focal laser injuries were created in the spinal cord (FIG. 2B), immediately post-injury, and imaged every 12 minutes for 10 hours. BELMO-expressing glia made substantially larger phagocytic cups than control (FIG. 2C). It was also found via live imaging that the speed of corpse uptake (duration for pulling in the phagocytic cup) is faster in BELMO-expressing cells than controls (FIG. 2D). Without injury, such phagocytic cup formation was not observed in either control or BELMO-positive glia, and the number of phagocytic cups generated was also comparable. Thus, BELMO can promote larger phagocytic cups and faster debris clearance by zebrafish glia in vivo.

To test BELMO function in vivo in mammals, BELMO transgenic mice were generated by inserting a single copy of the BELMO transgene into the Rosa26 locus in C57BL/6 mouse embryonic stem cells (BELMW^{flox-STOP-flox}) (see Methods). BELMO expression was basally kept silent by an upstream transcriptional-translational STOP cassette, and its removal via cell type-specific Cre was expected to induce expression of BELMO and the bicistronic eGFP marker (FIG. 2E). This was the case when the BELMO^{flox-STOP-flox} (BELMO^{Tg}) mice were crossed to Cx3cr1-cre mice (targeting primarily the monocytic/macrophage lineage). BELMO⁺ peritoneal macrophages, as tracked by their bicistronic eGFP expression, showed a significant increase in apoptotic cell uptake and greater corpse-derived fluorescence on a per cell basis compared to control macrophages ex vivo (FIG. 2F). BELMO⁺ peritoneal macrophages retained the classic anti-inflammatory responses associated with efferocytosis, as measured by IL-10 and Leukemia Inhibitory Factor (LIF) release after efferocytosis (FIG. S2B). As another approach, apoptotic Jurkat cells were injected directly into the peritoneum of Cx3cr1-cre.BELMO^{Tg} mice and measured efferocytic uptake by eGFP+(BELMO expressing) peritoneal macrophages in vivo. Again, eGFP⁺ large peritoneal macrophages (CD11b^{high} F4/80^{high}) in the Cx3cr1-cre.BELMO^{Tg} mice displayed significantly increased efferocytosis (FIG. 2G). These data established that BELMO expression can promote efferocytosis in vivo.

### BELMO Attenuates Disease Parameters in Multiple Models of Tissue Injury

Defective efferocytosis, leading to the accumulation of uncleared apoptotic cells and secondary necrosis, is associated with various inflammatory diseases (Boada-Romero et al., 2020; Doran et al., 2020; Lemke, 2019; Morioka et al., 2019; Nagata, 2018; Rothlin et al., 2021). Therefore, we tested whether expression of BELMO and improved efferocytosis could dampen inflammation and alleviate injury in tissue injury models affecting the gut, liver, and kidney.

To study BELMO in the context of gut injury, the dextran sulfate sodium (DSS)-induced colitis model was used, as epithelial cell-mediated clearance of neighboring apoptotic cells plays a role in dampening the DSS-induced colitis (Morioka et al., 2019). As a proof of principle, first it was tested whether expression of BELMO in HCT116, a colonic epithelial cell line, could enhance efferocytosis in vitro, which was indeed the case (FIG. 3A). BELMO^{Tg} mice were then crossed with Villin-cre mice to target BELMO expression to intestinal epithelial cells. Control and Villin-cre BELMO^{Tg} mice were treated with DSS for 7 days and multiple disease parameters were monitored (FIG. 3B). Compared to the control mice, the Villin-cre BELMO^{Tg} mice maintained colonic crypt architecture (FIG. 3C) and colon length (FIG. 3D). Next, the status of apoptotic cell accumulation in control and Villin-cre BELMO^{Tg} mice was assessed. While TUNEL⁺ cells accumulated in control mice, this was largely absent in Villin-cre BELMO^{Tg} mice (FIG. 3E). This was also confirmed by evaluating caspase-3⁺ activity (apoptosis) in the BELMO^{Tg} mice (FIG. S3A). BELMO expression did not alter apoptosis of epithelial cells when exposed to DSS in vitro (FIG. S3B). Additionally, early damage induced by DSS monitored by intestinal caspase-3 activity was comparable between Villin-cre and Villin-cre BELMO^{Tg} mice (FIG. S3C). Lastly, when inflammatory parameters were analyzed, less proinflammatory cytokine expression was detected in Villin-cre BELMO^{Tg} mice compared to control mice (FIG. 3F). Thus, BELMO expression in intestinal epithelial cells protected in the DSS-induced injury model.

BELMO was then tested in a hepatotoxicity model. BELMO^{Tg} mice were crossed with Alb-cre mice to express BELMO in hepatocytes. BELMO⁺ primary hepatocytes incubated with apoptotic Jurkat cells displayed greater efferocytosis ex vivo (FIG. 4A). Previous studies have shown that intraperitoneal injection of diethylnitrosamine (DEN) into mice induces rapid hepatotoxicity within 48 hours, and this involves hepatocyte apoptosis (FIG. 4B) (Maeda et al., 2005). Consistent with these reports, a substantial increase in TUNEL® cells and caspase 3 activity was found after 48 hours in control Alb-cre only mice; however, this was significantly attenuated in Alb-Cre BELMO^{Tg} mice (FIG. 4C and S4A). Plasma alanine aminotransferase (ALT), an indicator of liver injury and released from necrotic hepatocytes, was significantly lower in Alb-cre BELMO^{Tg} compared to control mice (FIG. 4C). Proinflammatory cytokine expressions were also considerably reduced in the BELMO transgenic mice compared Alb-Cre control mice (FIG. S4B). BELMO did not appear to affect apoptosis per se, BELMO expressing primary hepatocytes underwent apoptosis similar to control cells (FIG. S4C). These data suggested that BELMO expression protects in a second model of apoptosis-induced injury.

As a third model of inflammatory tissue injury, cisplatin-induced nephrotoxicity was used, where cisplatin, a widely used cancer chemotherapeutic agent, induces nephrotoxicity linked to apoptosis of kidney tubular epithelial cells (TEC) (Miller et al., 2010). TECs represent >60% of all cell types in the kidney, and previous studies have shown that TEC can phagocytose apoptotic cells (Arai et al., 2016; Ichimura et al., 2008). To target BELMO expression to the TEC, BELMO^{Tg} mice were crossed with PEPCK-cre mice. TECs from PEPCK-cre BELMO^{Tg} mice showed greater efferocytosis when fed with apoptotic Jurkat cells ex vivo than controls (FIG. 4D). Two days after cisplatin injection, significantly fewer TUNEL⁺ positive cells and cleaved caspase-3⁺ cells were detected in the PEPCK-cre BELMO^{Tg} mice (FIG. 4E, 4F, and S4D). This was seen despite the initial damage within the first 12 h after cisplatin treatment being comparable between PEPCK-cre and PEPCK-cre BELMO^{Tg} mice (FIG. S4E). When efferocytic TECs were scored using the ApoTag kit that measures DNase II-mediated DNA breaks (that occur in the phagolysosomes during digestion of apoptotic cells), more efferocytic TECs were noticed in the PEPCK-cre BELMO^{Tg} mice compared to controls (FIG. 4F). Compared to control mice, plasma creatinine levels and overall survival rate were significantly improved in PEPCK-cre BELMOTg mice (FIG. 4F). Further, when pro-inflammatory cytokine expression was measured after cisplatin treatment, PEPCK-cre BELMO^{Tg} mice had much less pro-inflammatory gene expression (FIG. S4F). Additionally, when Cx3cr1-cre BELMO^{Tg} were challenged with cisplatin, a protective effect as monitored by creatinine levels and mouse survival was not detected; thus, BELMO expression in the TEC was necessary for the protective effects (FIG. S4G).

Collectively, the data from three different models suggest that BELMO expression enhances efferocytosis by intestinal epithelial cells, hepatocytes, and kidney tubular epithelial cells in vivo, and this can, in turn, ameliorate tissue injury and inflammation.

### Protein Folding Modulators as New Players in Efferocytic Capacity of Phagocytes

During the above studies, BELMO-mediated efferocytosis were noticed. First, among a population of phagocytes, more of the BELMO expressing phagocytes performed efferocytosis; second, even in highly efficient phagocytes such as macrophages, BELMO expression led to more corpses engulfed on a per-cell basis; and third, when we used CellTraceViolet-labeled apoptotic targets to track corpse digestion within phagocytes, the acidification in BELMO⁺ phagocytes was comparable or even more efficient than control phagocytes despite the increased corpse load per phagocyte (FIGS. S1D). These observations suggested that besides being a more ‘potent’ receptor at the membrane that facilitates corpse internalization, BELMO-mediated signaling might also help overcome other ‘rate-limiting’ steps in efferocytosis. To understand this, an RNAseq of efferocytic BELMO⁺ phagocytes was performed using either non-professional phagocytes (fibroblasts) or professional phagocytes (macrophages) (FIG. 5A and S5A). These analyses were also useful from another perspective. Typically, when phagocytes engage and internalize apoptotic cells, multiple receptors on phagocytes are utilized; in the context of BELMO expressing cells, although the other receptors may still function, the enhanced efferocytosis is dominated by one signaling module, i.e., the ELMO/Dock180/Rac module (FIG. 1I).

Comparison of transcriptomic profiles of BELMO-expressing and control fibroblasts revealed ˜480 genes that were influenced by BELMO expression during efferocytosis; this included ˜326 genes that were unique to efferocytic BELMO-expressing cells and ˜150 genes that were altered both in control and BELMO⁺ phagocytes following efferocytosis (FIG. 5B). In gene ontology analysis of BELMO-dependent genes, one set of genes was striking, i.e., those linked to protein folding/endoplasmic reticulum (ER) function. The other pathways modulated in BELMO⁺ phagocytes provided validation of what is previously known in the literature, such as the upregulation of genes encoding proteins linked to aerobic glycolysis, TGFβ pathway, as well as downregulation of inflammatory responses such as the INFγ pathway, and cholesterol synthesis (FIG. 5C) (A-Gonzalez et al., 2017; Cummings et al., 2016; Morioka et al., 2019; Perry et al., 2019). In addition, when transcriptomic analysis was conducted of efferocytic BELMO-expressing and control peritoneal macrophages in vitro, upregulation of many genes linked to protein folding/endoplasmic reticulum (ER) function was noticed again (FIG. S5A). In closer analysis, many of these genes are associated with protein folding in the ER, degradation of unfolded proteins, and other ER functions (FIG. 5D). Although the genes linked to the so-called ‘unfolded protein’ responses (UPR) were initially characterized in the context of accumulation of misfolded proteins in the ER, many recent studies have firmly established that the UPR pathway genes impact the cellular protein homeostasis (or “proteostasis”) and chronic inflammation (Hipp et al., 2019; Inagi et al., 2014; Labbadia and Morimoto, 2015). It was surmised that as a phagocyte engulfing an apoptotic cell can double its cellular protein contents, with many phagocytes taking up multiple corpses, the protein homeostasis could be a rate-limiting step. The possibility that BELMO might help overcome this limitation was intriguing.

The effect of whether pharmacological disruption of protein homeostasis in control and BELMO⁺ phagocytes during efferocytosis was monitored. Calcium entry into the ER is linked to protein homeostasis; treating phagocytes with thapsigargin pretreatment, an inhibitor of ER calcium transport, led to a strong reduction in efferocytosis in control phagocytes. Although BELMO⁺ phagocytes are also sensitive to thapsigargin, they were more resistant than control phagocytes when tested in a dose-response (FIG. 5E). Consistent with this observation, the gene Atp2a3, which encodes one of the SERCA pumps inhibited by thapsigargin, was upregulated in BELMO⁺ efferocytic phagocytes (FIG. 5D and S5B).

As part of the cellular proteostasis, various ER-resident enzymes and chaperones increase protein folding efficiency. One of the most abundant proteins within the ER is the Hsp70-type chaperone BiP (also called GRP-78), the expression of which was significantly increased in efferocytic BELMO⁺ phagocytes (FIG. 5D and S5B). Following treatment with a BiP inhibitor, the enhanced efferocytosis by BELMO⁺ phagocytes was substantially attenuated (FIG. 5F). Conversely, promoting protein folding in control cells by a chemical chaperone, 4-Phenylbutyric acid (4-PBA), greatly enhanced their efferocytosis; however, 4-BPA only modestly increased efferocytosis by BELMO expressing fibroblasts (FIG. 5G). BiP has been shown to work cooperatively with DNAJC3, and DNAJC3 was also upregulated in BELMO⁺ efferocytic phagocytes. Lastly, Vimp, a molecule involved in the degradation of unfolded proteins, was also upregulated in BELMO⁺ phagocytes (FIG. 5D and S5B).

To complement the above pharmacological approaches, two genetic approaches were undertaken: first, siRNA knockdown of BiP, Dnajc3, Atp2a3, and Vimp blunted the BELMO-mediated enhanced efferocytosis; second, CRISPR/Cas9 mediated deletion BiP, Dnajc3, Atp2a3, and Vimp also attenuated the enhanced efferocytosis in BELMO-expressing phagocytes (FIG. 6A and S5C). Collectively, these data identify multiple genes linked to cellular proteostasis as rate-limiting regulators in the efferocytic capacity of phagocytes and that BELMO expressing phagocytes acquire an efferocytic advantage through improved proteostasis.

### Proteostasis Regulation Via BELMO Affects Kidney Injury Outcomes In Vivo

Defective proteostasis is highly deleterious to renal cell function and viability, with links to the pathophysiology of various kidney diseases (Cybulsky, 2017; Inagi et al., 2014; Shu et al., 2018; Yan et al., 2018). Dysregulation of proteostasis often occurs in the kidney under oxidative stress, glycative stress, or hypoxia conditions. A well-established model of acute kidney injury (AKI), linked to defective ER proteostasis, is the bilateral ischemia reperfusion injury (IRI) (FIG. 6B) (Cybulsky, 2017; Inagi et al., 2014; Shu et al., 2018; Yan et al., 2018). As TECs are one of the most susceptible cell types to the stressors to the kidney, we used PEPCK-cre BELMO^{Tg} mice for these studies. In the bilateral IRI model, the PEPCK-cre BELMO^{Tg} mice displayed a striking amelioration of disease after induction of AKI based on several parameters: first, creatinine level in the PEPCK-cre BELMO^{Tg} mice was significantly reduced compared to PEPCK-cre control mice (FIG. 6C); second, in the IRI protocol with longer ischemia times (i.e. more severe injury) PEPCK-cre BELMO^{Tg} mice showed greater viability, and also showed much improved health/activity (FIG. 6D and Video S1); third, while kidneys from control mice were severely damaged with disrupted tubular structure, the kidney from PEPCK-cre BELMO^{Tg} mice were largely protected (FIG. 6E). The initial damage observed within the first 6 h after bilateral IRI injury was comparable between the PEPCK-cre BELMO^{Tg} mice and PEPCK-cre control mice (FIG. S5D).

When asked whether the beneficial outcomes after AKI in PEPCK-cre BELMO^{Tg} mice correlate with BiP expression (as a marker of proteostasis), there was much greater BiP upregulation in the PEPCK-cre BELMO^{Tg} mice compared to control mice (FIG. 6F). To test whether the increased BiP expression contributed to the attenuated AKI, the PEPCK-cre BELMO^{Tg} mice were subjected to the AKI regimen, together with or without BiP inhibitor pre-treatment before the bilateral RI. The BiP inhibitor reversed the protective phenotype of the PEPCK-cre BELMO^{Tg} mice and increased the creatinine levels (FIG. 6G). These data suggest that the improved proteostasis in vitro in BELMO expressing phagocytes correlates to improved protection in ER-proteostasis AKI in vivo.

Next, it was tested whether overexpression of a chimeric efferocytosis receptor can help dampen inflammation in an ongoing/chronic disease model. For this, we chose a model of longer-term kidney injury associated with fibrosis. In this context, it was attempted to induce BELMO expression after disease induction using adeno associated virus (AAV) vectors, which are highly useful for gene delivery and used in various human disease trials. However, the large molecular size of the BAI1 component of BELMO posed technical difficulties in generating AAV vectors with BELMO. As the beneficial effects of BELMO depend on ELMO-mediated downstream signaling, it was asked whether one could fuse ELMO to an efferocytic receptor of smaller molecular size. TIM4 is a PtdSer receptor that does not have any apparent cytoplasmic signaling motifs but can potently promote efferocytosis (Miyanishi et al., 2007; Park et al., 2009). The TIM4 extracellular region was fused with the same part of ELMO as used in BELMO to generate ‘TELMO’ (FIG. 7A and S6A). TELMO was expressed on the cell surface and significantly increased efferocytosis of apoptotic cells through signaling via ELMO in vitro (FIGS. 7B and 7C).

To test whether TELMO expression in chronic kidney disease can improve outcomes, AAV9-TELMO was generated and injected this vector into the renal vein of the left kidney (FIG. 7D). In baseline experiments, it was established that it takes −7 days to obtain AAV9-based TELMO expression and that TELMO is detected primarily on the cell surface of tubular epithelial cells (SLC34A1⁺); further, TELMO was only detected on cells from the left kidney (that received the AAV-TELMO vector) but not the contralateral right kidney (FIG. 7E). Next, to assess the ability of TELMO to impact chronic kidney disease, unilateral ischemia reperfusion injury was performed on the AAV-TELMO (or control vector) injected left kidney, and then removed the contralateral (uninjured) right kidney 14 days later (FIG. 7D); this approach ‘forces’ the mouse to use/depend on the left kidney that had the IRI and received AAV-TELMO. When renal function was assessed after removing the compensatory right kidney, plasma creatinine and blood urea nitrogen (BUN) were significantly higher in the control mice than the AAV-TELMO expressing mice (FIG. 7F and S6B). Further, TELMO-AAV kidneys had much less collagen deposition and fibrosis, as detected via Masson's trichrome staining (FIG. 7G). As this chronic kidney injury model is also associated with disrupted proteostasis, it was asked whether the TELMO expression correlated with higher BiP expression. Indeed, 3-4-fold higher BiP expression was detected in TELMO expressing kidneys 14-days post-injury (FIG. 7H). TELMO expression was detected on day 7 “after” kidney injury was triggered (day 2), suggesting that TELMO could ameliorate ongoing kidney injury. Collectively, these data suggest that the expression of chimeric efferocytic receptors can improve disease outcomes in a model of chronic inflammation.

The extracellular region of TELMO was also mutated to abrogate PtdSer binding; Wi 19A, F120A, N121A, and D122A mutations were engineered in TIM4 (denoted TELMO^{4A}) While TELMO^{4A} was still expressed on the plasma membrane, unlike TELMO, TELMO^{4A} did not boost efferocytosis. TELMO^{4A} also did not provide a protective effect in the kidney IRI injury, suggesting that PtdSer recognition is necessary for the repair. In addition, plasma membrane-targeted ELMO1 (ELMO1 (532-727)-CAAX) without the TIM4 or BAI1 extracellular region also failed to reduce kidney injury.

### Discussion

Defective efferocytosis underlies a growing list of human diseases (Boada-Romero et al., 2020; Doran et al., 2020; Gregory and Pound, 2010; Henson, 2017; Lemke, 2019; Morioka et al., 2019; Nagata, 2018; Rothlin et al., 2021). The data presented in this manuscript advance several new concepts.

First, described herein is the engineering of a novel tool to improve efferocytosis in vivo. Specifically, using an intracellular domain of the engulfment protein ELMO1 that was previously defined as a domain for efferocytic signaling (necessary and sufficient for its interaction with Dock proteins and for activation of the downstream signaling pathway that involves the GTPase) and fusing this domain directly to PtdSer recognition receptors, chimeric receptors for efferocytosis (CHEF) were created. The PtdSer recognition via the extracellular domains of either BAI1 or TIM4 and the intracellular signaling via ELMO1 ensure specific recognition of apoptotic cells (not live cells) and strongly enhances efferocytosis. Further, as CHEF mediates the typical anti-inflammatory features of efferocytosis, this has the added benefit of promoting anti-inflammatory signaling by the efferocytic phagocytes at a much greater scale. BELMO and TELMO expressing phagocytes engulf more apoptotic cells, but they can also process the ingested corpses adequately.

Second, using BELMO^{Tg} mice with inducible expression tested in three different injury models in vivo, the CHEF approach dampened inflammation and associated disease parameters in DSS-induced colitis in the gut, hepatocyte-injury model in the liver, and cisplatin-induced nephrotoxicity. When initially engineered CHEF, it was uncertain how CHEF may function in professional versus non-professional phagocytes. It was a surprise that the CHEF approach boosts efferocytosis in professional phagocytes such as macrophages and non-professional phagocytes such as fibroblasts, epithelial cells, and glia in the transgenic zebrafish context.

Third, mechanistic studies using BELMO-expressing phagocytes via transcriptomic analyses complemented by siRNA-mediated knockdown/CRISPR mediated deletion/pharmacological approaches identify a previously unappreciated role for proteostasis as a rate-limiting step in efferocytosis. Specifically, BELMO increases the expression of proteostasis promoting genes such as BiP, DNAJC3, ATP2A3, and VIP; this, in turn, provides a functional efferocytic advantage to BELMO⁺ phagocytes such that they not only engulf more apoptotic corpses but manage the excess corpse load. This improved proteostasis in the BELMO-expressing phagocytes was functionally relevant in vivo, as evident in acute and chronic kidney injury models. Thus, it was posited that BELMO⁺ phagocytes are more potent in dampening inflammation in at least two ways: faster removal of the apoptotic cells (which will prevent their secondary necrosis) and improved proteostasis in the BELMO⁺ phagocytes that ingest more corpses on a per-cell basis.

Fourth, CHEF can improve outcomes in a model of ongoing chronic inflammation. Using TELMO as the CHEF and inducing its expression via adeno-associated viral vectors in an ongoing chronic kidney disease model improved the tissue function of these mice and decreased the fibrosis. This beneficial effect again correlated with improved proteostasis markers.

Over many years in the field of efferocytosis, a number of elegant studies have been performed, knocking out individual receptors or cytoplasmic molecules to demonstrate the importance of efferocytosis in initiating or sustaining inflammatory diseases. Interestingly, when single receptor knockouts are analyzed, they often show minimal defects in efferocytosis. This suggests redundancy among efferocytosis receptors, yet the sheer number of receptors linked to apoptotic cell recognition or facilitating the uptake of apoptotic cells also suggests they may play unique functions. Many of these engulfment receptors facilitate uptake and contribute to anti-inflammatory signaling, a hallmark of efferocytosis. Thus, during the design of CHEF, a cytoplasmic signaling module of ELMO that is widely expressed across cell types and evolution and linked to Rac-dependent cytoskeletal reorganization was chosen; however, it was unsure if this module would also be sufficient to provide the anti-inflammatory signaling within its phagocytes. The data presented in this work show that this ELMO domain used (which is smaller than most fluorescent tags such as GFP) may be tethered to other efferocytosis receptors as needed, with an expectation that it will have beneficial signaling effects. Thus, conceptually, this small ELMO domain may also be coupled to other receptors, which recognize molecules exposed under pro-inflammatory contexts, to dampen inflammation. Similarly, identifying ‘transposable’ PtdSer binding regions of BAI1 and TIM4 suggests that they may be coupled to pro-inflammatory cytoplasmic signaling entities to enhance immune responses against tumors that often harbor PtdSer⁺ apoptotic cells.

### Methods

### Mice

C57BL 6J, Cx3cr1-cre, Villin-cre, and Alb-Cre mice were purchased from the Jackson Laboratories. PEPCK-cre mice were provided by Volker Haase (Vanderbilt University) (Rankin et al., 2006).

### Cells

LR73 cells were derived by Jeffrey Pollard (Stanners et al., 1979). Jurkat and HCT116 cells were purchased from ATCC.

### In Vitro Engulfment Assay

For induction of apoptosis, human Jurkat T cells resuspended in RPMI with 1% BSA were treated with 150 mJ/cm² ultraviolet C irradiation (Stratalinker) and incubated for 4 h at 37° C., 5% CO₂, and the cells were then stained with CypHer5E (GE Healthcare, PA15401) or CellTrace Violet (Thermo Fisher Scientific) before use in the engulfment assays. For phosphatidylserine masking to block efferocytosis, Jurkat cells were incubated with recombinant Annexin V protein at 3 g/ml for 30 min (eBioscience). Chinese hamster LR73 cells or murine macrophages were seeded in a 24-well plate and incubated with targets at a 1:10 phagocyte to target ratio for the indicated times. Unbound/unengulfed targets were then washed with PBS. After the indicated incubation times, cells were dissociated from the plate with trypsin, and assessed by a flow cytometry-based assay or prepared for analysis of RNA.

### Microscopy Analysis

For phagocytosis imaging, LR73 cells were seeded in 500 μl of αMEM at 2×10⁴ cells per well of a 24-well treated tissue culture plate (Falcon) and cultured for 24 h. Thymocytes were isolated, and apoptosis was induced by 20 μM dexamethasone for 4 h; cells were labeled with the pH-sensitive dye CypHer 5E (1 μM) in cation-free Hanks balanced salt solution for 20 minutes before adding to LR73 cells. The plate was then mounted on a stage-top environmental chamber of a Nikon Ti-Eclipse inverted microscope to maintain 37° C./5% carbon dioxide throughout the experiment. Phase contrast and fluorescent images were captured using a 40× objective for the indicated time. Images were analyzed using Image J version 2.1.0.

### Immunoblotting, Immunoprecipitation and Flow Cytometry

LR73 cells were seeded in a 100 mm dish at a concentration of 2 million cells/dish. Apoptotic Jurkat cells were added as indicated. Cells were lysed in RIPA buffer and immunoblotted for BAI1 (R&D, MAB4969), ELMO1 (Brugnera et al., 2002), Rac1 (Cytoskeleton, ARC03), and total Erk2 (Santa Cruz Biotechnology, #sc-154-G) antibodies in Can Get Signal solution (TOYOBO Cat #NKB-101) followed by chemiluminescence detection. For immunoprecipitation, 1 mg of lysate was incubated with 1 μg of Dock180 antibody (Santa Cruz 6047 or 6167) overnight for immunoblotting. Plasma membrane expression of BELMO and TELMO was confirmed by BAI1 (R&D, MAB4969) and TIM4 (Biolegend, 130002). Tubular epithelial cells were detected by SLC34A1 antibody (G-Biosciences, ITN1348).

### Generation of Transgenic Zebrafish

All constructs were generated using the Tol2kit Gateway-based cloning system (Kwan et al., 2007). Vectors used for making the expression constructs were p5E-slc1a3b(−9.5) (Chen et al., 2020), p5E-dA-nls-mCherry-biTRE, pME-rtTA-HA (Campbell et al., 2012), pME-BELMO-GFP, pME-eGFP, p3E-polyA, pDestTol2pA2, pDestTol2CG2 (Kwan et al., 2007), and pDestTol2pACryGFP destination vectors (Berger and Currie, 2013). To build pME-BELMO-GFP, BELMO-GFP was subcloned into the pME-MCS entry vector (Kwan et al., 2007) to generate a pME vector for Gateway cloning. p5E, pME and p3E-polyA expression vectors were ligated into destination vectors via LR reactions (Ashton et al., 2012). One-cell stage embryos were injected with 2nl plasmid DNAs at a concentration of 20 ng/L, combined with 10 ng/L Tol2 transposase mRNA. Larvae were selected based on the expression of reporter genes. Slc1a3b:rtTA-HA larvae were identified by expression of cmcl2:eGFP, which results in eGFP expression in heart cells. Larvae carrying nls-mCherry-biTRE-BELMO-GFP were identified by expression of cry:eGFP, resulting in eGFP expression in the eyes. For induction of BELMO expression, 3 dpf larvae were treated with 10 μM doxycycline/1% DMSO in PTU egg water for 24 hours prior to imaging. Control siblings were treated with 1% DMSO in PTU egg water. Embryos were anesthetized with 0.01% 3-aminobenzoic acid ester (Tricaine), immersed in 0.8% low-melting point agarose and mounted in glass-bottomed 35 mm Petri dishes (Electron Microscopy Sciences). After mounting, the Petri dish was filled with egg water containing PTU, Tricaine and respective drug treatments. A 40× water objective (NA=1.1) mounted on a motorized Zeiss AxioObserver Z1 microscope equipped with a Quorum WaveFX-XI (Quorum Technologies) spinning disc confocal system was used to capture all images using Metamorph software. Focal injury was created with a 435-nm pulsed nitrogen dye laser which was pulsed in small circular regions of interest (ROIs) around the glia, followed by time-lapse imaging. Images were taken every 12 minutes for 10 hours. Injury was confirmed by brightfield. Images were processed with ImageJ/Fiji and pseudo-colored for better visualization of fine processes. Any drift in the images was corrected by the Fiji plugin “Correct 3D drift”. Further image alteration was limited to levels and contrast. Phagocytic cups from n=8 and n=12 cells from n=8 and n=8 different fish were quantified for BELMO-negative and BELMO-positive cells, respectively.

### Generation of Transgenic Mouse

To generate conditional BELMO transgenic mice, BELMO cDNA was inserted into the previously described CAG-STOP-eGFP-ROSA26TV (CTV) vector (Lee et al., 2016). CTV-BELMO vector was transfected into C57BL/6 embryonic stem cells (JM8A3) and screened for homologous recombination into the Rosa26 locus. Induced expression of transgene encoded BELMO and eGFP were confirmed in the ES cells in vitro via Cre transfection. Selected ES clones were then used for blastocyst injections by the UVA mouse core to obtain transgenic mice. BELMO^{flox-STOP-flox} mice were crossed with E2A-cre mice (Jackson laboratory Stock No: 003724) for global transgene expression, CX3CR1-cre mice (Jackson laboratory Stock No: 025524) for myeloid expression, Vill-cre mice (Jackson laboratory Stock No: 004586) for intestinal epithelial cell expression, Alb-cre mice (Jackson laboratory Stock No: 003574) for hepatocyte expression and PEPCK-CRE (Rankin et al., 2006) for tubular epithelial cell expression. All animal experiments were performed according to protocols approved by the animal care and use committee (ACUC) at the University of Virginia.

### In Vivo Engulfment Assay

Six million apoptotic Jurkat cells, stained with CypHer5E, were intraperitoneally injected in 300p1 volume per mouse, or as control, X-VIVO 10 medium alone. At indicated times post-injection, mice were euthanized, and the peritoneal lavage was collected by 10 mL of PBS+10% FBS. The collected cells were stained with CD11b PE-Cy7 (eBioscience, Cat #: 25-0112-82) and F4/80 APC-eFluor 780 (eBioscience, Cat #: 47-4801-80), and the uptake of the injected CypHer5E positive apoptotic cells by CD11b⁺ F4/80^{hi} cells was assessed by flow cytometry.

### Luminex Assessment of Cytokine Production

For in vitro experiments, phagocytes were cultured in 6-well plate with apoptotic cells for 2 h. Unbound apoptotic cells were then removed by 3×PBS wash, 1 ml of fresh media was added, and phagocytes were cultured an additional 12 h. Supernatant was then collected in 1.5 ml Eppendorf tube, spun to collect cells and debris, then transferred to a fresh tube for downstream Luminex analysis.

### DSS-Induced Colitis Model

Mice (littermates, 8˜10 weeks old) were given 3% dextran sulfate sodium (DSS; MP Biomedicals) in drinking water for indicated days and analyzed. Changes in body weight and disease severity index were checked daily. Disease severity was determined based on weight loss, blood in the stool, and stool consistency, as previously described (Lee et al., 2016). The total scores were calculated as follows: weight loss (0: <1%, 1: 1˜5%, 2: 6˜10%, 3: 11˜15%, 4: >15%), stool blood (0: negative, 2: positive, 4: gross bleeding), and stool consistency (0: normal, 2: loose stools, 4: diarrhea).

### Flow Cytometry for Apoptosis

Apoptotic cells were stained with annexin V-Pacific Blue and 7AAD for 15 min at room temperature in annexin V binding buffer (140 mM NaCl, 2.5 M CaCl, 10 mM HEPES) and subjected to flow cytometry. Data were analyzed using FlowJo v.10 software.

### Hepatocyte Isolation and Analysis in the DEN-Induced Hepatotoxicity Model

Primary hepatocytes were isolated with a standard collagenase procedure according to the manufacturer's instructions (Lonza). Diethylnitrosamine (100 mg/kg) dissolved in corn oil was administered by intraperitoneal injection. 48 h later, mice were euthanized, plasma was collected, and ALT was measured according to the manufacturer's instructions (Biovision). Mice were euthanized after treatments and tissues were isolated for histology analysis.

### Cisplatin-Induced Nephrotoxicity Model

Cisplatin (20 mg/kg or 25 mg/kg) dissolved in 0.9% normal saline was administered by intraperitoneal injection. 72 h later, the mice were euthanized under anesthesia by cervical dislocation. Plasma was prepared by centrifuging heparinized blood at 4,800 g for 5 minutes. Plasma creatinine (mg/dl) was determined using an enzymatic method, with minor modifications from the manufacturer's protocol (using twice the recommended volume of sample and 2-fold serial dilution of the calibrator [standard] provided in the kit; Diazyme Laboratories). We validated the enzymatic kit by comparing with analysis of creatinine by liquid chromatography-mass spectrometry (LC-MS) performed at the George F. O'Brien Center (University of Alabama, Birmingham, Alabama, USA).

### Histology Staining and Analysis

Mice were euthanized after treatments, and depending on the disease model, the colon, liver, or kidney was isolated. Proximal colon region was cut by microtome at 200 μm intervals. TUNEL staining was conducted according to the manufacturer's instructions (Invitrogen). Cleaved caspase-3 staining and Masson's trichrome staining were performed at the University of Virginia Biorepository and Tissue Research Facility and Research Histology Core, respectively. Engulfed apoptotic cells were stained by labeling DNA breaks from DNase type II within phagolysosome using Apoptag ISOL dual fluorescence apoptosis detection kit (Millipore). The nuclei were counterstained with Hoechst 33342. The samples were analyzed using Axiolmager Z2 equipped with Apotome for optical sectioning.

Colon sections were assessed in a blinded study in microscopy-based scoring using the following parameters: Severity of inflammation was scored as follows: 0, rare inflammatory cells in the lamina propria (<10%); 1, mild (10-25%); 2, moderate (26-50%); and 3, severe (>51%). The extent of inflammatory cell infiltration was scored as follows: 0, normal; 1, mucosal infiltration; 2, submucosal infiltration; or 3, transmural infiltration. Crypt damage or erosion was scored as follows: 0, normal; 1, mild; 2, moderate; or 3, severe. The extent of acute tubular necrosis (ATN) and kidney tubulointerstitial fibrosis revealed by trichrome staining were assessed in an unbiased, systematic manner using design-based stereology to achieve statistically accurate random sampling of kidney sections. The investigator was blinded to the experimental identity of the sections. Sections were imaged by using a Zeiss Axio Imager Z2/Apotome Microscope fitted with motorized focus drives and a motorized XYZ microscope stage (MBF Bioscience) and integrated to a workstation running Stereo Investigator Morioka et al, Page No. 21software (version 11.06.2, MBF Bioscience). The area fraction fractionator probe (Stereo Investigator) was used for stereological analysis of the fractional area (percentage of total surface area) of the section occupied by acute tubular necrosis or tubulointerstitial collagen deposition (Masson trichrome stain). The following parameters were defined: counting frame, 400×400 m; sample grid, 800×800 m; and grid spacing, 85 m. These values were determined empirically such that adequate numbers of sample sites were visited, in keeping with accepted counting rules for stereology. A total of 625±20 (mean SEM) grid sites were evaluated per section

### RNA Sequencing and Bioinformatics

LR73 cells and primary peritoneal macrophages with or without BELMO expression were co-cultured with apoptotic Jurkat cells for 2 h, unbound Jurkat cells removed by washing with PBS, and the phagocytes were rested in culture medium for an additional 2 h. Total RNA was extracted, and the mRNA library was prepared using the Illumina TruSeq platform, followed by sequencing using an Illumina NextSeq 500 cartridge. Four independent experiments were sequenced. R v.4.0.3 was used for graphical and statistical analysis and the R package DESeq2 were used for count normalization and differential gene expression analysis of RNA-seq data. Gene set enrichment analysis (GSEA) was performed using the R package fgsea. Heatmaps were created using the R package heatmap3.

### Kidney Ischemia Reperfusion Injury (IRI)

Mice were anesthetized via intraperitoneal injection of ketamine (120 mg/kg) and xylazine (12 mg/kg) and were then subjected to bilateral or unilateral kidney IRI. Briefly, IRI was performed through flank incisions by clamping the renal pedicles for indicated times. The clamps were then removed, and the wound was sutured after restoration of blood flow was visually observed. Sham-operated mice underwent the same procedure except that the renal pedicles were not clamped. Mice received buprenorphine SR (0.5 mg/kg) as a postoperative analgesic. Kidneys were allowed to reperfuse for the indicated times. For unilateral IRI, 1×10¹² AAV-TELMO in 75 μl PBS or control were injected into renal vein during clamp; 14 days after reperfusion, contralateral right kidney was removed to evaluate the function of the left kidney. Blood urea nitrogen (BUN) was measured using the DetectX Urea Nitrogen (BUN) Detection kit (Arbor Assays, Ann Arbor, MI). Creatinine measurement is described above. The mice were euthanized with an overdose of ketamine and xylazine, and blood was collected from the retroorbital sinus. The kidneys were harvested for histology and RNA extraction. All mouse procedures were performed by approved IACUC protocols.

### Quantitative RT-PCR

Total RNA was extracted from cells using RNeasy Mini Kit (Qiagen) and cDNA was synthesized using QuantiTect Reverse Transcription Kit (Qiagen) according to manufacturers' instructions. Quantitative gene expression for hamster and mouse genes was performed using Taqman probes (Applied Biosystems) run on a StepOnePlus Real-Time PCR System (Applied Biosystems).

siRNA knockdown

For siRNA experiments, LR73 cells were treated with Lipofectamine 2000 (ThermoFisher) with specific siRNAs according to the manufacturer's instructions 2 days before the engulfment assay. Non-targeting control siRNA #1 and targeting siRNAs were customized by Horizon Discovery.

siRNA Against Bip:

(SEQ ID NO: 1)
5′---AGAAGGAACUAGAGGAAAUUU---3′

Two siRNAs Against Dnajc3:

(SEQ ID NO: 2)
5′---GCUAGGAGACCAUGAGUUAUU---3′ 3′

Two siRNAs Against Atp2a3:

(SEQ ID NO: 3)
5′---CAACAAAGGCACAGCUGUAUU---3′ 3′

Two siRNAs Against Vimp:

(SEQ ID NO: 4)
5′---GCUAAGACAGCUUGAAGAAUU---3′ 3′

### CRISPR-Cas9 Deletion

Stable iCas9-GFP-expressing LR73 cells were generated using Lenti-iCas9-Neo via lentiviral transduction (Cao et al., 2016). Genes were deleted from LR73 cells using lentiGuide-Puro sgRNA plasmid. Lenti-iCas9-neo was a gift from Qin Yan (Addgene plasmid #85400) and lentiGuide-Puro was a gift from Feng Zhang (Addgene plasmid #52963).

Non-targeting guide RNA was generated using the following oligonucleotide pair.

(SEQ ID NO: 5)
5′---CACCGGAATGGCGTACGATTCGCG---3′ 3′
(SEQ ID NO: 6)
3′---CCTTACCGCATGCTAAGCGCCAAA---5′ 3′.

Guide RNA targeting BiP was generated using the following oligonucleotide pair.

(SEQ ID NO: 7)
5′---CACCGCGGTGGTCGGCATCGACCTG---3′ 3′
(SEQ ID NO: 8)
3′---GCCACCAGCCGTAGCTGGACCAAA---5′ 3′.

Guide RNA targeting Dnajc3 was generated using the following oligonucleotide pair.

(SEQ ID NO: 9)
5′---CACCGGAATGGCGTACGATTCGCG---3′ 3′
(SEQ ID NO: 10)
3′---CGACTTCTCGCCATTGTGGCCAAA---5′ 3′.

Guide RNA targeting Atp2a3 was generated using the following oligonucleotide pair.

(SEQ ID NO: 11)
5′---CACCGGGTACCACGTATACCCCTG---3′ 3′
(SEQ ID NO: 12)
3′---CCCATGGTGCATATGGGGACCAAA---5′ 3′.

Guide RNA targeting Vimp was generated using the following oligonucleotide pair.

(SEQ ID NO: 13)
5′---CACCGCGCGCGGACAGAGGTTCCT---3′ 3′
(SEQ ID NO: 14)
3′---CGCGCGCCTGTCTCCAAGGACAAA---5′ 3′.

### Adenovirus Production

TELMO-GFP construct was subcloned into the pAAV-MCS Expression Vector (VPK-410, CELL BIOLABS, INC.) using the EcoRI restriction site. Packaging, purification and titration of AAV9-TELMO were performed by Vigene Biosciences, Inc.

### Statistical Analysis

Statistical significance was determined using GraphPad Prism 9, using unpaired Student's two-tailed t-test, one-way ANOVA, or two-way ANOVA according to test requirements. Grubbs' Outlier Test was used to determine outliers, which were excluded from final analysis. A p value of <0.05 (indicated by one asterisk), <0.01 (indicated by two asterisks), or <0.001 (indicated by three asterisks) were considered significant.

### BIBLIOGRAPHY

A-Gonzalez, et al. (2017). Phagocytosis imprints heterogeneity in tissue-resident macrophages. J Exp Med 214, 1281-1296.
Arai, S., et al. (2016). Apoptosis inhibitor of macrophage protein enhances intraluminal debris clearance and ameliorates acute kidney injury in mice. Nat Med 22, 183-193.
Ashton, R. S., et al. (2012). Astrocytes regulate adult hippocampal neurogenesis through ephrin-B signaling. Nat Neurosci 15, 1399-1406.
Berger, J., and Currie, P. D. (2013). 503unc, a small and muscle-specific zebrafish promoter. Genesis 51, 443-447.
Boada-Romero, et al. (2020). The clearance of dead cells by efferocytosis. Nat Rev Mol Cell Biol 21, 398-414.
Brugnera, E., et al. (2002). Unconventional Rac-GEF activity is mediated through the Dock180-ELMO complex. Nat Cell Biol 4, 574-582.
Campbell, L. J., et al. (2012). Two types of Tet-On transgenic lines for doxycycline-inducible gene expression in zebrafish rod photoreceptors and a gateway-based tet-on toolkit. PLoS One 7, e51270.
Cao, J., et al. (2016). An easy and efficient inducible CRISPR/Cas9 platform with improved specificity for multiple gene targeting. Nucleic Acids Res 44, e149.
Chang, L., et al. (2020). Structure of the DOCK2-ELMO1 complex provides insights into regulation of the auto-inhibited state. Nat Commun 11, 3464.
Chen, J., et al. (2020). Live-imaging of astrocyte morphogenesis and function in zebrafish neural circuits. Nat Neurosci 23, 1297-1306.
Cummings, R. J., et al. (2016). Different tissue phagocytes sample apoptotic cells to direct distinct homeostasis programs. Nature 539, 565-569.
Cybulsky, A. V. (2017). Endoplasmic reticulum stress, the unfolded protein response and autophagy in kidney diseases. Nat Rev Nephrol 13, 681-696. deBakker, C. D., et al. (2004). Phagocytosis of apoptotic cells is regulated by a UNC-73/TRIO-MIG-2/RhoG signaling module and armadillo repeats of CED-12/ELMO. Curr Biol 14, 2208-2216.
Doran, A. C., et al. (2020). Efferocytosis in health and disease. Nat Rev Immunol 20, 254-267.
Elliott, M. R., and Ravichandran, K. S. (2016). The Dynamics of Apoptotic Cell Clearance. Dev Cell 38, 147-160.
Elliott, M. R., et al. (2010). Unexpected requirement for ELMO1 in clearance of apoptotic germ cells in vivo. Nature 467, 333-337.
Epting, D., et al. (2010). The Rac1 regulator ELMO1 controls vascular morphogenesis in zebrafish. Circ Res 107, 45-55.
Fourgeaud, L., et al. (2016). TAM receptors regulate multiple features of microglial physiology. Nature 532, 240-244.
Geisbrecht, E. R., et al. (2008). Drosophila ELMO/CED-12 interacts with Myoblast city to direct myoblast fusion and ommatidial organization. Dev Biol 314, 137-149.
Gregory, C. D., and Pound, J. D. (2010). Microenvironmental influences of apoptosis in vivo and in vitro. Apoptosis 15, 1029-1049.
Grimsley, C. M., et al. (2004). Dock180 and ELMO1 proteins cooperate to promote evolutionarily conserved Rac-dependent cell migration. J Biol Chem 279, 6087-6097.
Gumienny, T. L., et al. (2001). CED-12/ELMO, a novel member of the CrkII/Dock180/Rac pathway, is required for phagocytosis and cell migration. Cell 107, 27-41.
Han, C. Z., et al. (2016). Macrophages redirect phagocytosis by non-professional phagocytes and influence inflammation. Nature 539, 570-574.
Henson, P. M. (2017). Cell Removal: Efferocytosis. Annu Rev Cell Dev Biol 33, 127-144.
Henson, P. M., and Hume, D. A. (2006). Apoptotic cell removal in development and tissue homeostasis. Trends Immunol 27, 244-250.
Hipp, M. S., et al. (2019). The proteostasis network and its decline in ageing. Nat Rev Mol Cell Biol 20, 421-435.
Ichimura, T., et al. (2008). Kidney injury molecule-1 is a phosphatidylserine receptor that confers a phagocytic phenotype on epithelial cells. J Clin Invest 118, 1657-1668.
Inagi, R., et al. (2014). Proteostasis in endoplasmic reticulum—new mechanisms in kidney disease. Nat Rev Nephrol 10, 369-378.
Juncadella, I. J., et al. (2013). Apoptotic cell clearance by bronchial epithelial cells critically influences airway inflammation. Nature 493, 547-551.
Kwan, K. M., et al. (2007). The Tol2kit: a multisite gateway-based construction kit for Tol2 transposon transgenesis constructs. Dev Dyn 236, 3088-3099.
Labbadia, J., and Morimoto, R. I. (2015). The biology of proteostasis in aging and disease. Annu Rev Biochem 84, 435-464.
Lee, C. S., et al. (2016). Boosting Apoptotic Cell Clearance by Colonic Epithelial Cells Attenuates Inflammation In vivo. Immunity 44, 807-820.
Lemke, G. (2019). How macrophages deal with death. Nature Reviews Immunology 19, 539-549.
Lu, M., et al. (2004). PH domain of ELMO functions in trans to regulate Rac activation via Dock180. Nat Struct Mol Biol 11, 756-762.
Maeda, S., et al. (2005). IKKbeta couples hepatocyte death to cytokine-driven compensatory proliferation that promotes chemical hepatocarcinogenesis. Cell 121, 977-990.
Miller, R. P., et al. (2010). Mechanisms of Cisplatin nephrotoxicity. Toxins (Basel) 2, 2490-2518.
Miyanishi, M., et al. (2007). Identification of Tim4 as a phosphatidylserine receptor. Nature 450, 435-439.
Monks, J., et al. (2008). Epithelial cells remove apoptotic epithelial cells during post-lactation involution of the mouse mammary gland. Biol Reprod 78, 586-594.
Morioka, S., et al. (2019). Living on the Edge: Efferocytosis at the Interface of Homeostasis and Pathology. Immunity 50, 1149-1162.
Nagata, S. (2018). Apoptosis and Clearance of Apoptotic Cells. Annu Rev Immunol 36, 489-517.
Park, D., et al. (2009). The phosphatidylserine receptor TIM-4 does not mediate direct signaling. Curr Biol 19, 346-351.
Park, D., et al. (2007). BAI1 is an engulfment receptor for apoptotic cells upstream of the ELMO/Dock180/Rac module. Nature 450, 430-434.
Patel, M., et al. (2010). An evolutionarily conserved autoinhibitory molecular switch in ELMO proteins regulates Rac signaling. Curr Biol 20, 2021-2027.
Perry, J. S. A., et al. (2019). Interpreting an apoptotic corpse as anti-inflammatory involves a chloride sensing pathway. Nat Cell Biol 21, 1532-1543.
Rankin, E. B., et al. (2006). Renal cyst development in mice with conditional inactivation of the von Hippel-Lindau tumor suppressor. Cancer Res 66, 2576-2583.
Rothlin, C. V., et al. (2021). Determining the effector response to cell death. Nat Rev Immunol 21, 292-304.
Shankman, L. S., et al. (2021). Efferocytosis by Paneth cells within the intestine. Curr Biol 31, 2469-2476 e2465.
Shu, S. Q., et al. (2018). Endoplasmic reticulum stress is activated in post-ischemic kidneys to promote chronic kidney disease. Ebiomedicine 37, 269-280.
Tosello-Trampont, A. C., et al. (2007). Identification of two signaling submodules within the CrkII/ELMO/Dock180 pathway regulating engulfment of apoptotic cells. Cell Death Differ 14, 963-972.
Yan, M., et al. (2018). Endoplasmic reticulum stress in ischemic and nephrotoxic acute kidney injury. Ann Med 50, 381-390.
Yanagihashi, Y., et al. (2017). Mouse macrophages show different requirements for phosphatidylserine receptor Tim4 in efferocytosis. Proc Natl Acad Sci USA 114, 8800-8805.
Zhu, D., et al. (2015). BAI1 regulates spatial learning and synaptic plasticity in the hippocampus. J Clin Invest 125, 1497-1508.

### Example II

By ringing BAI1 and ELMO1 together inducibly one could test whether one can recreate the gain of function seen with BELMO. For this, the rapamycin-based inducible dimerization approach was used. BAI1-FRB and ELMO-FKBP fusion constructs were generated to allow for rapamycin-dependent dimerization. While expression of either BAI1-FRB or ELMO-FKBP alone did not lead to greater efferocytosis, inducing the interaction of BAI-FRB and ELMO1-FKBP, via addition of rapamycin, greatly increased efferocytosis (FIG. S7B). Thus, both the BAI1 and ELMO components are necessary.

All publications, patents, and patent applications, Genbank sequences, websites and other published materials referred to throughout the disclosure herein are herein incorporated by reference to the same extent as if each individual publication, patent, or patent application, Genbank sequences, websites and other published materials was specifically and individually indicated to be incorporated by reference. In the event that the definition of a term incorporated by reference conflicts with a term defined herein, this specification shall control.

1. A fusion protein comprising an intracellular domain of engulfment protein ELMO1 and an extracellular phosphatidylserine recognition domain of an efferocytic receptor.

2. The fusion protein of claim 1, wherein the efferocytic receptor is BAI1 or TIM4.

3. A RNA or DNA sequence coding for the fusion protein of claim 1.

4. A transgenic cell or transgenic non-human animal comprising a transgene which codes for and expresses the fusion protein of claim 1.

5. The transgenic cell or transgenic non-human animal of claim 4, wherein the transgene is stably or transiently expressed.

6. The transgenic cell or transgenic non-human animal of claim 4, wherein the cell is a phagocyte.

7. The transgenic cell or transgenic non-human animal of claim 6, wherein the phagocyte is a professional phagocyte.

8. The transgenic cell or transgenic non-human animal of claim 7, wherein the professional phagocyte is a macrophage.

9. The transgenic cell or transgenic non-human animal of claim 6, wherein the phagocyte is a non-professional phagocyte.

10. The transgenic cell or transgenic non-human animal of claim 9, wherein the non-professional phagocyte is an epithelial cell, fibroblast, glial cell or endothelial cell.

11. The transgenic cell or transgenic non-human animal of claim 6, wherein the animal is a zebrafish or a mouse.

12. A method to increase efferocytosis/phagocytosis of apoptotic cells in vitro and/or in vivo comprising contacting a cell or administering to a subject in need thereof the fusion protein of claim 1.

13-21. (canceled)

22. The method of claim 12, wherein the subject has a pathological condition.

23. The method of claim 22, wherein the pathological condition is selected from the group consisting of including tissue injury, infection, neurodegenerative diseases, and autoimmune diseases.

24. (canceled)

25. A method to treat kidney disease comprising administering to a subject in need thereof the fusion protein of claim 1.

26. The method of claim 25, wherein after administration plasma creatinine, blood urea nitrogen (BUN) or combination thereof is decreased compared to prior to administration.

27-29. (canceled)

30. The method of claim 12, wherein RNA is administered.

31. The method of claim 12, wherein DNA is administered.

32. The method of claim 31, wherein the DNA is part of an expression vector.

33. The method of claim 32, wherein the expression vector is a viral vector.

34-37. (canceled)


SEQUENCE LISTING (29 sequences)

SEQ ID NO: 1 - RNA, 21 nt, synthetic construct
  source 1..21: mol_type = other RNA
agaaggaactagaggaaattt

SEQ ID NO: 2 - RNA, 21 nt, synthetic construct
  source 1..21: mol_type = other RNA
gctaggagaccatgagttatt

SEQ ID NO: 3 - RNA, 21 nt, synthetic construct
  source 1..21: mol_type = other RNA
caacaaaggcacagctgtatt

SEQ ID NO: 4 - RNA, 21 nt, synthetic construct
  source 1..21: mol_type = other RNA
gctaagacagcttgaagaatt

SEQ ID NO: 5 - RNA, 24 nt, synthetic construct
  source 1..24: mol_type = other RNA
caccggaatggcgtacgattcgcg

SEQ ID NO: 6 - RNA, 24 nt, synthetic construct
  source 1..24: mol_type = other RNA
ccttaccgcatgctaagcgccaaa

SEQ ID NO: 7 - RNA, 25 nt, synthetic construct
  source 1..25: mol_type = other RNA
caccgcggtggtcggcatcgacctg

SEQ ID NO: 8 - RNA, 24 nt, synthetic construct
  source 1..24: mol_type = other RNA
gccaccagccgtagctggaccaaa

SEQ ID NO: 9 - RNA, 24 nt, synthetic construct
  source 1..24: mol_type = other RNA
caccggaatggcgtacgattcgcg

SEQ ID NO: 10 - RNA, 24 nt, synthetic construct
  source 1..24: mol_type = other RNA
cgacttctcgccattgtggccaaa

SEQ ID NO: 11 - RNA, 24 nt, synthetic construct
  source 1..24: mol_type = other RNA
caccgggtaccacgtatacccctg

SEQ ID NO: 12 - RNA, 24 nt, synthetic construct
  source 1..24: mol_type = other RNA
cccatggtgcatatggggaccaaa

SEQ ID NO: 13 - RNA, 24 nt, synthetic construct
  source 1..24: mol_type = other RNA
caccgcgcgcggacagaggttcct

SEQ ID NO: 14 - protein, 24 aa, synthetic construct
CGCGCGCCTGTCTCCAAGGACAAA

SEQ ID NO: 15 - DNA, 2336 bp, synthetic construct
  source 1..2336: mol_type = other DNA
tgtccaaggggcttctcctcctctggctggtgacggagctctggtggctttatctgacaccagctgcctcagaggatacaataatagggtttttgggccagccggtgactttgccttgtcattacctctcgtggtcccagagccgcaacagtatgtgctggggcaaaggttcatgtcccaattccaagtgcaatgcagagcttctccgtacagatggaacaagaatcatctccaggaagtcaacaaaatatacacttttggggaaggtccagtttggtgaagtgtccttgaccatctcaaacaccaatcgaggtgacagtggggtgtactgctgccgtatagaggtgcctggctggttcaatgatgtcaagaagaatgtgcgcttggagctgaggagagccacaacaaccaaaaaaccaacaacaaccacccggccaaccaccaccccttatgtgaccaccaccaccccagagctgcttccaacaacagtcatgaccacatctgttctcccaaccaccacaccaccccagacactagccaccactgccttcagtacagcagtgaccacgtgcccctcaacaacacctggctccttctcacaagaaaccacaaaagggtccgccttcactacagaatcagaaactctgcctgcatccaatcactctcaaagaagcatgatgaccatatctacagacatagccgtactcaggcccacaggctctaaccctgggattctcccatccacttcacagctgacgacacagaaaacaacattaacaacaagtgagtctttgcagaagacaactaaatcacatcagatcaacagcagacagaccatcttgatcattgcctgctgtgtgggatttgtgctaatggtgttattgtttctggcgtttctccttcgagggaaagtcacaggagccaactgtttgcagagacacaagaggccagacaacactgaagatagtgacagcgtccatcatcaccatcaccattcccgggttccgattttggaactaaaggagaagatccagccagaaatcttagagctgattaaacagcagcgcctgaaccgccttgtggaagggacctgctttaggaaactcaatgctcgccggagacaagacaagttttggtattgtcggctttcaccaaatcacaaggtcttacattatggcgacttggaagagagcccccaaggagaagtgccccacgattccctgcaggacaaattgccggtggcagatatcaaagcagtggtgacgggaaaggactgccctcatatgaaagagaaaggtgcccttaaacaaaacaaggaggtgcttgaacttgctttctccatcttatacgactcaaattgccaactgaacttcattgctcctgataagcatgagtactgcatctggacagatgggctgaatgcactgcttgggaaggacatgatgagtgacctgacacgcaatgacctggacaccctgctgagcatggagatcaagcttcgcctgctggacctggaaaacatccagatcccggatgcacctccgcctatccccaaggaacctagcaactatgactttgtctatgactgtaacacccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 16 - protein, 778 aa, synthetic construct
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLLRGKVTGANCLQRHKRPDNTEDSDSVHHHHHHSRVPILELKEKIQPEILELIKQQRLNRLVEGTCFRKLNARRRQDKFWYCRLSPNHKVLHYGDLEESPQGEVPHDSLQDKLPVADIKAVVTGKDCPHMKEKGALKQNKEVLELAFSILYDSNCQLNFIAPDKHEYCIWTDGLNALLGKDMMSDLTRNDLDTLLSMEIKLRLLDLENIQIPDAPPPIPKEPSNYDFVYDCNTRDPPVATMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 17 - DNA, 5064 bp, synthetic construct
  source 1..5064: mol_type = other DNA
atgaggggccaggccgccgccccgggccccgtctggatcctcgccccgctgctactgctgctgctgctgctgggacgccgcgcgcgggcggccgccggagcagacgcggggcccgggcccgagccgtgcgccacgctggtgcagggaaagttcttcggctacttctccgcggccgccgtgttcccggccaacgcctcgcgctgctcctggacgctacgcaacccggacccgcggcgctacactctctacatgaaggtggccaaggcgcccgtgccctgcagcggccccggccgcgtgcgcacctaccagttcgactccttcctcgagtccacgcgcacctacctgggcgtggagagcttcgacgaggtgctgcggctctgcgacccctccgcacccctggccttcctgcaggccagcaagcagttcctgcagatgcggcgccagcagccgccccagcacgacgggctccggccccgggccgggccgccgggccccaccgacgacttctccgtggagtacctggtggtggggaaccgcaaccccagccgtgccgcctgccagatgctgtgccgctggctggacgcgtgtctggccggtagtcgcagctcgcacccctgcgggatcatgcagaccccctgcgcctgcctgggcggcgaggcgggcggccctgccgcgggacccctggccccccgcggggatgtctgcttgagagatgcggtggctggtggccctgaaaactgcctcaccagcctgacccaggaccggggcgggcacggcgccacaggcggctggaagctgtggtccctgtggggcgaatgcacgcgggactgcgggggaggcctccagacgcggacgcgcacctgcctgcccgcgccgggcgtggagggcggcggctgcgagggggtgctggaggagggtcgccagtgcaaccgcgaggcctgcggccccgctgggcgcaccagctcccggagccagtccctgcggtccacagatgcccggcggcgcgaggagctgggggacgagctgcagcagtttgggttcccagccccccagaccggtgacccagcagccgaggagtggtccccgtggagcgtgtgctccagcacctgcggcgagggctggcagacccgcacgcgcttctgcgtgtcctcctcctacagcacgcagtgcagcggacccctgcgcgagcagcggctgtgcaacaactctgccgtgtgcccagtgcatggtgcctgggatgagtggtcgccctggagcctctgctccagcacctgtggccgtggctttcgggatcgcacgcgcacctgcaggcccccccagtttgggggcaacccctgtgagggccctgagaagcaaaccaagttctgcaacattgccctgtgccctggccgggcagtggatggaaactggaatgagtggtcgagctggagcgcctgctccgccagctgctcccagggccgacagcagcgcacgcgtgaatgcaacgggccttcctacgggggtgcggagtgccagggccactgggtggagacccgagactgcttcctgcagcagtgcccagtggatggcaagtggcaggcctgggcgtcatggggcagttgcagcgtcacgtgtggggctggcagccagcgacgggagcgtgtctgctctgggcccttcttcgggggagcagcctgccagggcccccaggatgagtaccggcagtgcggcacccagcggtgtcccgagccccatgagatctgtgatgaggacaactttggtgctgtgatctggaaggagaccccagcgggagaggtggctgctgtccggtgtccccgcaacgccacaggactcatcctgcgacggtgtgagctggacgaggaaggcatcgcctactgggagccccccacctacatccgctgtgtttccattgactacagaaacatccagatgatgacccgggagcacctggccaaggctcagcgagggctgcctggggagggggtctcggaggtcatccagacactggtggagatctctcaggacgggaccagctacagtggggacctgctgtccaccatcgatgtcctgaggaacatgacagagattttccggagagcgtactacagccccacccctggggacgtacagaactttgtccagatccttagcaacctgttggcagaggagaatcgggacaagtgggaggaggcccagctggcgggccccaacgccaaggagctgttccggctggtggaggactttgtggacgtcatcggcttccgcatgaaggacctgagggatgcataccaggtgacagacaacctggttctcagcatccataagctcccagccagcggagccactgacatcagcttccccatgaagggctggcgggccacgggtgactgggccaaggtgccagaggacagggtcactgtgtccaagagtgtcttctccacggggctgacagaggccgatgaagcatccgtgtttgtggtgggcaccgtgctctacaggaacctgggcagcttcctggccctgcagaggaacacgaccgtcctgaattctaaggtgatctccgtgactgtgaaacccccgcctcgctccctgcgcacacccttggagatcgagtttgcccacatgtataatggcaccaccaaccagacctgtatcctgtgggatgagacggatgtaccctcctcctccgcccccccgcagctcgggccctggtcgtggcgcggctgccgcacggtgcccctcgacgccctccggacgcgctgcctctgtgaccggctctccaccttcgccatcttagcccagctcagcgccgacgcgaacatggagaaggcgactctgccgtcggtgacgctcatcgtgggctgtggcgtgtcctctctcaccctgctcatgctggtcatcatctacgtgtccgtgtggaggtacattcgctcagagcgttctgtcatcctcatcaacttctgcctgtccatcatctcctccaatgccctcatcctcatcgggcagacccagacccgcaacaaggtgatgtgcacgctggtggccgccttcctgcacttcttcttcctgtcctccttctgctgggtgctcaccgaggcctggcagtcctacatggccgtgacgggccacctccggaaccgcctcatccgcaagcgcttcctctgcctgggctgggggctccctgcactggttgtggccatttctgtgggattcaccaaggccaaagggtacagcaccatgaactactgctggctctccctggaggggggactgctctatgccttcgtgggacctgccgctgccgttgtgctggtgaacatggtcattgggatcctggtgttcaacaagctcgtgtccaaagacggcatcacggacaagaagctgaaggagcgggcaggggcctccctgtggagctcctgcgtggtgctgccgctgctggcgctgacctggatgtcggctgtgctcgccgtcaccgaccgccgctccgccctcttccagatcctcttcgctgtcttcgactcgctggagggcttcgtcatcgtcatggtgcactgtatcctccgtagagaggtccaggacgctgtgaaatgccgtgtggttgaccggcaggaggagggcaacggggactcagggggctccttccagaacggccacgcccagctcatgaccgacttcgagaaggacgtgcatcatcaccatcaccatgtcgagccgattttggaactaaaggagaagatccagccagaaatcttagagctgattaaacagcagcgcctgaaccgccttgtggaagggacctgctttaggaaactcaatgctcgccggagacaagacaagttttggtattgtcggctttcaccaaatcacaaggtcttacattatggcgacttggaagagagcccccaaggagaagtgccccacgattccctgcaggacaaattgccggtggcagatatcaaagcagtggtgacgggaaaggactgccctcatatgaaagagaaaggtgcccttaaacaaaacaaggaggtgcttgaacttgctttctccattttatacgactcaaattgccaactgaacttcattgctcctgataagcatgagtactgcatctggacagatgggctgaatgcactgcttgggaaggacatgatgagtgacctgacacgcaatgacctggacaccctgctgagcatggagatcaagcttcgcctgctggacctggaaaacatccagatcccggatgcacctccgcctatccccaaggaacctagcaactatgactttgtctatgactgtaacgtcgacggaggggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 18 - protein, 1687 aa, synthetic construct
MRGQAAAPGPVWILAPLLLLLLLLGRRARAAAGADAGPGPEPCATLVQGKFFGYFSAAAVFPANASRCSWTLRNPDPRRYTLYMKVAKAPVPCSGPGRVRTYQFDSFLESTRTYLGVESFDEVLRLCDPSAPLAFLQASKQFLQMRRQQPPQHDGLRPRAGPPGPTDDFSVEYLVVGNRNPSRAACQMLCRWLDACLAGSRSSHPCGIMQTPCACLGGEAGGPAAGPLAPRGDVCLRDAVAGGPENCLTSLTQDRGGHGATGGWKLWSLWGECTRDCGGGLQTRTRTCLPAPGVEGGGCEGVLEEGRQCNREACGPAGRTSSRSQSLRSTDARRREELGDELQQFGFPAPQTGDPAAEEWSPWSVCSSTCGEGWQTRTRFCVSSSYSTQCSGPLREQRLCNNSAVCPVHGAWDEWSPWSLCSSTCGRGFRDRTRTCRPPQFGGNPCEGPEKQTKFCNIALCPGRAVDGNWNEWSSWSACSASCSQGRQQRTRECNGPSYGGAECQGHWVETRDCFLQQCPVDGKWQAWASWGSCSVTCGAGSQRRERVCSGPFFGGAACQGPQDEYRQCGTQRCPEPHEICDEDNFGAVIWKETPAGEVAAVRCPRNATGLILRRCELDEEGIAYWEPPTYIRCVSIDYRNIQMMTREHLAKAQRGLPGEGVSEVIQTLVEISQDGTSYSGDLLSTIDVLRNMTEIFRRAYYSPTPGDVQNFVQILSNLLAEENRDKWEEAQLAGPNAKELFRLVEDFVDVIGFRMKDLRDAYQVTDNLVLSIHKLPASGATDISFPMKGWRATGDWAKVPEDRVTVSKSVFSTGLTEADEASVFVVGTVLYRNLGSFLALQRNTTVLNSKVISVTVKPPPRSLRTPLEIEFAHMYNGTTNQTCILWDETDVPSSSAPPQLGPWSWRGCRTVPLDALRTRCLCDRLSTFAILAQLSADANMEKATLPSVTLIVGCGVSSLTLLMLVIIYVSVWRYIRSERSVILINFCLSIISSNALILIGQTQTRNKVMCTLVAAFLHFFFLSSFCWVLTEAWQSYMAVTGHLRNRLIRKRFLCLGWGLPALVVAISVGFTKAKGYSTMNYCWLSLEGGLLYAFVGPAAAVVLVNMVIGILVFNKLVSKDGITDKKLKERAGASLWSSCVVLPLLALTWMSAVLAVTDRRSALFQILFAVFDSLEGFVIVMVHCILRREVQDAVKCRVVDRQEEGNGDSGGSFQNGHAQLMTDFEKDVHHHHHHVEPILELKEKIQPEILELIKQQRLNRLVEGTCFRKLNARRRQDKFWYCRLSPNHKVLHYGDLEESPQGEVPHDSLQDKLPVADIKAVVTGKDCPHMKEKGALKQNKEVLELAFSILYDSNCQLNFIAPDKHEYCIWTDGLNALLGKDMMSDLTRNDLDTLLSMEIKLRLLDLENIQIPDAPPPIPKEPSNYDFVYDCNVDGGVPRARDPPVATMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 19 - protein, 1584 aa, Homo sapiens
MRGQAAAPGPVWILAPLLLLLLLLGRRARAAAGADAGPGPEPCATLVQGKFFGYFSAAAVFPANASRCSWTLRNPDPRRYTLYMKVAKAPVPCSGPGRVRTYQFDSFLESTRTYLGVESFDEVLRLCDPSAPLAFLQASKQFLQMRRQQPPQHDGLRPRAGPPGPTDDFSVEYLVVGNRNPSRAACQMLCRWLDACLAGSRSSHPCGIMQTPCACLGGEAGGPAAGPLAPRGDVCLRDAVAGGPENCLTSLTQDRGGHGATGGWKLWSLWGECTRDCGGGLQTRTRTCLPAPGVEGGGCEGVLEEGRQCNREACGPAGRTSSRSQSLRSTDARRREELGDELQQFGFPAPQTGDPAAEEWSPWSVCSSTCGEGWQTRTRFCVSSSYSTQCSGPLREQRLCNNSAVCPVHGAWDEWSPWSLCSSTCGRGFRDRTRTCRPPQFGGNPCEGPEKQTKFCNIALCPGRAVDGNWNEWSSWSACSASCSQGRQQRTRECNGPSYGGAECQGHWVETRDCFLQQCPVDGKWQAWASWGSCSVTCGAGSQRRERVCSGPFFGGAACQGPQDEYRQCGTQRCPEPHEICDEDNFGAVIWKETPAGEVAAVRCPRNATGLILRRCELDEEGIAYWEPPTYIRCVSIDYRNIQMMTREHLAKAQRGLPGEGVSEVIQTLVEISQDGTSYSGDLLSTIDVLRNMTEIFRRAYYSPTPGDVQNFVQILSNLLAEENRDKWEEAQLAGPNAKELFRLVEDFVDVIGFRMKDLRDAYQVTDNLVLSIHKLPASGATDISFPMKGWRATGDWAKVPEDRVTVSKSVFSTGLTEADEASVFVVGTVLYRNLGSFLALQRNTTVLNSKVISVTVKPPPRSLRTPLEIEFAHMYNGTTNQTCILWDETDVPSSSAPPQLGPWSWRGCRTVPLDALRTRCLCDRLSTFAILAQLSADANMEKATLPSVTLIVGCGVSSLTLLMLVIIYVSVWRYIRSERSVILINFCLSIISSNALILIGQTQTRNKVMCTLVAAFLHFFFLSSFCWVLTEAWQSYMAVTGHLRNRLIRKRFLCLGWGLPALVVAISVGFTKAKGYSTMNYCWLSLEGGLLYAFVGPAAAVVLVNMVIGILVFNKLVSKDGITDKKLKERAGASLWSSCVVLPLLALTWMSAVLAVTDRRSALFQILFAVFDSLEGFVIVMVHCILRREVQDAVKCRVVDRQEEGNGDSGGSFQNGHAQLMTDFEKDVDLACRSVLNKDIAACRTATITGTLKRPSLPEEEKLKLAHAKGPPTNFNSLPANVSKLHLHGSPRYPGGPLPDFPNHSLTLKRDKAPKSSFVGDGDIFKKLDSELSRAQEKALDTSYVILPTATATLRPKPKEEPKYSIHIDQMPQTRLIHLSTAPEASLPARSPPSRQPPSGGPPEAPPAQPPPPPPPPPPPPQQPLPPPPNLEPAPPSLGDPGEPAAHPGPSTGPSTKNENVATLSVSSLERRKSRYAELDFEKIMHTRKRHQDMFQDLNRKLQHAAEKDKEVLGPDSKPEKQQTPNKRPWESLRKAHGTPTWVKKELEPLQPSPLELRSVEWERSGATIPLVGQDIIDLQTEV

SEQ ID NO: 20 - RNA, 5535 nt, Homo sapiens
  source 1..5535: mol_type = other RNA
ggactttagaagccgttgctgccctctctgtcacctgaagcggggccctctcccatcccacccttgccccgcctccctgcccccaccgggccggccctgcccgccgccggaccctggcatgtcaagacctggtccgcgcctgcctgcccagcccgcggaaccccggcggccccgcgagctaggatgaggggccaggccgccgccccgggccccgtctggatcctcgccccgctgctactgctgctgctgctgctgggacgccgcgcgcgggcggccgccggagcagacgcggggcccgggcccgagccgtgcgccacgctggtgcagggaaagttcttcggctacttctccgcggccgccgtgttcccggccaacgcctcgcgctgctcctggacgctacgcaacccggacccgcggcgctacactctctacatgaaggtggccaaggcgcccgtgccctgcagcggccccggccgcgtgcgcacctaccagttcgactccttcctcgagtccacgcgcacctacctgggcgtggagagcttcgacgaggtgctgcggctctgcgacccctccgcacccctggccttcctgcaggccagcaagcagttcctgcagatgcggcgccagcagccgccccagcacgacgggctccggccccgggccgggccgccgggccccaccgacgacttctccgtggagtacctggtggtggggaaccgcaaccccagccgtgccgcctgccagatgctgtgccgctggctggacgcgtgtctggccggtagtcgcagctcgcacccctgcgggatcatgcagaccccctgcgcctgcctgggcggcgaggcgggcggccctgccgcgggacccctggccccccgcggggatgtctgcttgagagatgcggtggctggtggccctgaaaactgcctcaccagcctgacccaggaccggggcgggcacggcgccacaggcggctggaagctgtggtccctgtggggcgaatgcacgcgggactgcgggggaggcctccagacgcggacgcgcacctgcctgcccgcgccgggcgtggagggcggcggctgcgagggggtgctggaggagggtcgccagtgcaaccgcgaggcctgcggccccgctgggcgcaccagctcccggagccagtccctgcggtccacagatgcccggcggcgcgaggagctgggggacgagctgcagcagtttgggttcccagccccccagaccggtgacccagcagccgaggagtggtccccgtggagcgtgtgctccagcacctgcggcgagggctggcagacccgcacgcgcttctgcgtgtcctcctcctacagcacgcagtgcagcggacccctgcgcgagcagcggctgtgcaacaactctgccgtgtgcccagtgcatggtgcctgggatgagtggtcgccctggagcctctgctccagcacctgtggccgtggctttcgggatcgcacgcgcacctgcaggcccccccagtttgggggcaacccctgtgagggccctgagaagcaaaccaagttctgcaacattgccctgtgccctggccgggcagtggatggaaactggaatgagtggtcgagctggagcgcctgctccgccagctgctcccagggccgacagcagcgcacgcgtgaatgcaacgggccttcctacgggggtgcggagtgccagggccactgggtggagacccgagactgcttcctgcagcagtgcccagtggatggcaagtggcaggcctgggcgtcatggggcagttgcagcgtcacgtgtggggctggcagccagcgacgggagcgtgtctgctctgggcccttcttcgggggagcagcctgccagggcccccaggatgagtaccggcagtgcggcacccagcggtgtcccgagccccatgagatctgtgatgaggacaactttggtgctgtgatctggaaggagaccccagcgggagaggtggctgctgtccggtgtccccgcaacgccacaggactcatcctgcgacggtgtgagctggacgaggaaggcatcgcctactgggagccccccacctacatccgctgtgtttccattgactacagaaacatccagatgatgacccgggagcacctggccaaggctcagcgagggctgcctggggagggggtctcggaggtcatccagacactggtggagatctctcaggacgggaccagctacagtggggacctgctgtccaccatcgatgtcctgaggaacatgacagagattttccggagagcgtactacagccccacccctggggacgtacagaactttgtccagatccttagcaacctgttggcagaggagaatcgggacaagtgggaggaggcccagctggcgggccccaacgccaaggagctgttccggctggtggaggactttgtggacgtcatcggcttccgcatgaaggacctgagggatgcataccaggtgacagacaacctggttctcagcatccataagctcccagccagcggagccactgacatcagcttccccatgaagggctggcgggccacgggtgactgggccaaggtgccagaggacagggtcactgtgtccaagagtgtcttctccacggggctgacagaggccgatgaagcatccgtgtttgtggtgggcaccgtgctctacaggaacctgggcagcttcctggccctgcagaggaacacgaccgtcctgaattctaaggtgatctccgtgactgtgaaacccccgcctcgctccctgcgcacacccttggagatcgagtttgcccacatgtataatggcaccaccaaccagacctgtatcctgtgggatgagacggatgtaccctcctcctccgcccccccgcagctcgggccctggtcgtggcgcggctgccgcacggtgcccctcgacgccctccggacgcgctgcctctgtgaccggctctccaccttcgccatcttagcccagctcagcgccgacgcgaacatggagaaggcgactctgccgtcggtgacgctcatcgtgggctgtggcgtgtcctctctcaccctgctcatgctggtcatcatctacgtgtccgtgtggaggtacattcgctcagagcgttctgtcatcctcatcaacttctgcctgtccatcatctcctccaatgccctcatcctcatcgggcagacccagacccgcaacaaggtgatgtgcacgctggtggccgccttcctgcacttcttcttcctgtcctccttctgctgggtgctcaccgaggcctggcagtcctacatggccgtgacgggccacctccggaaccgcctcatccgcaagcgcttcctctgcctgggctgggggctccctgcactggttgtggccatttctgtgggattcaccaaggccaaagggtacagcaccatgaactactgctggctctccctggaggggggactgctctatgccttcgtgggacctgccgctgccgttgtgctggtgaacatggtcattgggatcctggtgttcaacaagctcgtgtccaaagacggcatcacggacaagaagctgaaggagcgggcaggggcctccctgtggagctcctgcgtggtgctgccgctgctggcgctgacctggatgtcggctgtgctcgccgtcaccgaccgccgctccgccctcttccagatcctcttcgctgtcttcgactcgctggagggcttcgtcatcgtcatggtgcactgtatcctccgtagagaggtccaggacgctgtgaaatgccgtgtggttgaccggcaggaggagggcaacggggactcagggggctccttccagaacggccacgcccagctcatgaccgacttcgagaaggacgtggatctggcctgtagatcagtgctgaacaaggacatcgcggcctgccgcactgccaccatcacgggcacactgaagcggccgtctctgcccgaggaggagaagctgaagctggcccatgccaaggggccgcccaccaatttcaacagcctgccggccaacgtgtccaagctgcacctgcacggctcaccccgctatcccggcgggcccctgcccgacttccccaaccactcactgaccctcaagagggacaaggcgcccaagtcctccttcgtcggtgacggggacatcttcaagaagctggactcggagctgagccgggcccaggagaaggctctggacacgagctacgtgatcctgcccacggccacggccacgctgcggcccaagcccaaggaggagcccaagtacagcatccacattgaccagatgccgcagacccgcctcatccacctcagcacggcccccgaggccagcctccccgcccgcagcccgccctcccgccagccccccagcggcgggccccccgaggcaccccctgcccagcccccaccgcctccgcccccaccgccaccacctccccagcagcccctgcccccaccgcccaatctggagccggcaccccccagcctgggggatcccggggagcctgccgcccatccgggacccagcacggggcccagcaccaagaacgagaatgtcgccaccttgtctgtgagctccctggagcggcggaagtcgcggtatgcagaactggactttgagaagatcatgcacacccggaagcggcaccaagacatgttccaggacctgaaccggaagctgcagcacgcagcggagaaggacaaggaggtgctggggccggacagcaagccggaaaagcagcagacgcccaacaagaggccctgggagagcctccggaaagcccacgggacgcccacgtgggtgaagaaggagctggagccgctgcagccgtcgccgctggagcttcgcagcgtggagtgggagaggtcgggcgccacgatcccgctggtgggccaggacatcatcgacctccagaccgaggtctgagcgggtgggcggcggccacgcactgggccacggaggagggatgctgctccgcccgctcctgccgcagacgggcacagacacgctcgcgggcagcgggccaggcccgcaccccggcctcagggcgctcagacggcggccaggcacagggcccgcagtgctgggaccagagccagatgcaggacaggaggcggcccggccagcgggcacagggcaccagaggccgaaggtgcctcagactccgccctcctcgggccgaggcccagcgggcagatgggcggacggctgtggaccgtggacaggcccagcgcggccagcgtcccagggtacccgcctgagctcctgctgcggaggagctgcctgcttggcccggccggcctggcaccgttttttaaacacccccatccctcgggaagcagccagctccccacaccttccagggccctaggcccctcctagacccaggtggagggcacagccctccgaccctcatggcccccaggggcaggactgagtcccctccaggaagaagcaggggggaatctattttttctctccttttcttttcttcaataaaaagaattaaaaacccaaaaaaaa

SEQ ID NO: 21 - protein, 727 aa, Homo sapiens
MPPPADIVKVAIEWPGAYPKLMEIDQKKPLSAIIKEVCDGWSLANHEYFALQHADSSNFYITEKNRNEIKNGTILRLTTSPAQNAQQLHERIQSSSMDAKLEALKDLASLSRDVTFAQEFINLDGISLLTQMVESGTERYQKLQKIMKPCFGDMLSFTLTAFVELMDHGIVSWDTFSVAFIKKIASFVNKSAIDISILQRSLAILESMVLNSHDLYQKVAQEITIGQLIPHLQGSDQEIQTYTIAVINALFLKAPDERRQEMANILAQKQLRSIILTHVIRAQRAINNEMAHQLYVLQVLTFNLLEDRMMTKMDPQDQAQRDIIFELRRIAFDAESEPNNSSGSMEKRKSMYTRDYKKLGFINHVNPAMDFTQTPPGMLALDNMLYFAKHHQDAYIRIVLENSSREDKHECPFGRSSIELTKMLCEILKVGELPSETCNDFHPMFFTHDRSFEEFFCICIQLLNKTWKEMRATSEDFNKVMQVVKEQVMRALTTKPSSLDQFKSKLQNLSYTEILKIRQSERMNQEDFQSRPILELKEKIQPEILELIKQQRLNRLVEGTCFRKLNARRRQDKFWYCRLSPNHKVLHYGDLEESPQGEVPHDSLQDKLPVADIKAVVTGKDCPHMKEKGALKQNKEVLELAFSILYDSNCQLNFIAPDKHEYCIWTDGLNALLGKDMMSDLTRNDLDTLLSMEIKLRLLDLENIQIPDAPPPIPKEPSNYDFVYDCN

SEQ ID NO: 22 - RNA, 2186 nt, Homo sapiens
  source 1..2186: mol_type = other RNA
caatgccgccacccgcggacatcgtcaaggtggccatagaatggccgggcgcctaccccaaactcatggaaattgatcagaaaaaaccactgtctgcaataataaaggaagtctgtgatgggtggtctcttgccaaccatgaatattttgcactccagcatgccgatagttcaaacttctatatcacagaaaagaaccgcaatgagataaaaaatggcactatccttcgattaaccacatctccagctcagaacgcccagcagctccatgaacgaatccagtcctcgagtatggatgccaagctggaagccctgaaggacttggccagcctctcccgggatgtcacgtttgcccaggagtttataaacctggacggtatctctctcctcacgcagatggtggagagcggcactgagcgataccagaaattgcagaagatcatgaagccttgctttggagacatgctgtccttcaccctgacggccttcgtcgagctgatggaccatggcatagtgtcctgggatacattttcggtggcgttcattaagaagatagcaagttttgtgaacaagtcagccatagacatctcgatcctgcagcggtccttggccattttggagtcgatggtgctcaatagccatgatctctaccagaaagtggcgcaggagatcaccatcggccagctcattccacacctgcaagggtcagatcaagaaatccaaacctatactattgcagtgattaatgcgcttttcctgaaggctcctgatgagaggaggcaggagatggcgaatattttggctcagaagcaactgcgttccatcattttaacacatgtcatccgagcccagcgggccatcaacaatgagatggcgcaccagctgtatgttctacaagtgctcacctttaacctcctggaagacaggatgatgaccaaaatggacccccaggaccaggctcagagggacatcatatttgaacttcgaagaattgcttttgatgctgagtctgaacctaacaacagcagtggcagcatggagaaacgcaagtccatgtacacgcgagattataagaagcttgggttcattaatcatgtcaaccctgccatggacttcacgcagactccacctgggatgttggctctggacaacatgctgtactttgccaagcaccaccaagatgcctacatccggattgtgcttgagaacagtagtcgagaagacaagcatgaatgtccctttggccgcagtagtatagagctgaccaagatgctatgtgagatcttgaaagtgggcgagttgcctagtgagacctgcaacgacttccacccgatgttcttcacccacgacagatcctttgaggagtttttctgcatctgtatccagctcctgaacaagacatggaaggaaatgagggcaacttctgaagacttcaacaaggtaatgcaggtggtgaaggagcaggttatgagagcacttacaaccaagcctagctccctggaccagttcaagagcaaactgcagaacctgagctacactgagatcctgaaaatccgccagtccgagaggatgaaccaggaagatttccagtcccgcccgattttggaactaaaggagaagattcagccagaaatcttagagctgatcaaacagcaacgcctgaaccgccttgtggaagggacctgctttaggaaactcaatgcccggcggaggcaagacaagttttggtattgtcggctttcgccaaatcacaaagtcctgcattacggagacttagaagagagtcctcagggagaagtgccccacgattccttgcaggacaaactgccggtggcagatatcaaagccgtggtgacgggaaaggactgccctcatatgaaagagaaaggtgcccttaaacaaaacaaggaggtgcttgaactcgctttctccatcttgtatgactcaaactgccaactgaacttcatcgctcctgacaagcatgagtactgtatctggacggatggactgaatgcgctactcgggaaggacatgatgagcgacctgacgcggaatgacctggacaccctgctcagcatggaaatcaagctccgcctcctggacctggaaaacatccagatccctgacgcacctccgccgattcccaaggagcccagcaactatgacttcgtctatgactgtaactga

SEQ ID NO: 23 - protein, 378 aa, Homo sapiens
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLLMIIAPSLGFVLFALFVAFLLRGKLMETYCSQKHTRLDYIGDSKNVLNDVQHGREDEDGLFTL

SEQ ID NO: 24 - RNA, 1330 nt, Homo sapiens
  source 1..1330: mol_type = other RNA
agactcctgggtccggtcaaccgtcaaaatgtccaaagaacctctcattctctggctgatgattgagttttggtggctttacctgacaccagtcacttcagagactgttgtgacggaggttttgggtcaccgggtgactttgccctgtctgtactcatcctggtctcacaacagcaacagcatgtgctgggggaaagaccagtgcccctactccggttgcaaggaggcgctcatccgcactgatggaatgagggtgacctcaagaaagtcagcaaaatatagacttcaggggactatcccgagaggtgatgtctccttgaccatcttaaaccccagtgaaagtgacagcggtgtgtactgctgccgcatagaagtgcctggctggttcaacgatgtaaagataaacgtgcgcctgaatctacagagagcctcaacaaccacgcacagaacagcaaccaccaccacacgcagaacaacaacaacaagccccaccaccacccgacaaatgacaacaaccccagctgcacttccaacaacagtcgtgaccacacccgatctcacaaccggaacaccactccagatgacaaccattgccgtcttcacaacagcaaacacgtgcctttcactaaccccaagcacccttccggaggaagccacaggtcttctgactcccgagccttctaaggaagggcccatcctcactgcagaatcagaaactgtcctccccagtgattcctggagtagtgttgagtctacttctgctgacactgtcctgctgacatccaaagagtccaaagtttgggatctcccatcaacatcccacgtgtcaatgtggaaaacgagtgattctgtgtcttctcctcagcctggagcatctgatacagcagttcctgagcagaacaaaacaacaaaaacaggacagatggatggaatacccatgtcaatgaagaatgaaatgcccatctcccaactactgatgatcatcgccccctccttgggatttgtgctcttcgcattgtttgtggcgtttctcctgagagggaaactcatggaaacctattgttcgcagaaacacacaaggctagactacattggagatagtaaaaatgtcctcaatgacgtgcagcatggaagggaagacgaagacggcctttttaccctctaacaacgcagtagcatgttagattgaggatgggggcatgacactccagtgtcaaaataagtcttagtagatttccttgtttcataaaaaagactcacttattccatggatgtcattgatccaggcttgctttagtttcatgaatgaagggtactttagagaccacaa

SEQ ID NO: 25 - protein, 350 aa, Homo sapiens
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKASDTAVPEQNKTTKTGQMDGIPMSMKNEMPISQLLMIIAPSLGFVLFALFVAFLLRGKLMETYCSQKHTRLDYIGDSKNVLNDVQHGREDEDGLFTL

SEQ ID NO: 26 - RNA, 1246 nt, Homo sapiens
  source 1..1246: mol_type = other RNA
agactcctgggtccggtcaaccgtcaaaatgtccaaagaacctctcattctctggctgatgattgagttttggtggctttacctgacaccagtcacttcagagactgttgtgacggaggttttgggtcaccgggtgactttgccctgtctgtactcatcctggtctcacaacagcaacagcatgtgctgggggaaagaccagtgcccctactccggttgcaaggaggcgctcatccgcactgatggaatgagggtgacctcaagaaagtcagcaaaatatagacttcaggggactatcccgagaggtgatgtctccttgaccatcttaaaccccagtgaaagtgacagcggtgtgtactgctgccgcatagaagtgcctggctggttcaacgatgtaaagataaacgtgcgcctgaatctacagagagcctcaacaaccacgcacagaacagcaaccaccaccacacgcagaacaacaacaacaagccccaccaccacccgacaaatgacaacaaccccagctgcacttccaacaacagtcgtgaccacacccgatctcacaaccggaacaccactccagatgacaaccattgccgtcttcacaacagcaaacacgtgcctttcactaaccccaagcacccttccggaggaagccacaggtcttctgactcccgagccttctaaggaagggcccatcctcactgcagaatcagaaactgtcctccccagtgattcctggagtagtgttgagtctacttctgctgacactgtcctgctgacatccaaagcatctgatacagcagttcctgagcagaacaaaacaacaaaaacaggacagatggatggaatacccatgtcaatgaagaatgaaatgcccatctcccaactactgatgatcatcgccccctccttgggatttgtgctcttcgcattgtttgtggcgtttctcctgagagggaaactcatggaaacctattgttcgcagaaacacacaaggctagactacattggagatagtaaaaatgtcctcaatgacgtgcagcatggaagggaagacgaagacggcctttttaccctctaacaacgcagtagcatgttagattgaggatgggggcatgacactccagtgtcaaaataagtcttagtagatttccttgtttcataaaaaagactcacttattccatggatgtcattgatccaggcttgctttagtttcatgaatgaagggtactttagagaccacaa

SEQ ID NO: 27 - protein, 299 aa, Homo sapiens
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGEMSSHHVAQAGLKSLGLK

SEQ ID NO: 28 - protein, 323 aa, Homo sapiens
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAASDTAVPEQNKTTKTGQMDGIPMSMKNEMPISQLLMIIAPSLGFVLFALFVAFLLRGKLMETYCSQKHTRLDYIGDSKNVLNDVQHGREDEDGLFTL

SEQ ID NO: 29 - RNA, 1165 nt, Homo sapiens
  source 1..1165: mol_type = other RNA
agactcctgggtccggtcaaccgtcaaaatgtccaaagaacctctcattctctggctgatgattgagttttggtggctttacctgacaccagtcacttcagagactgttgtgacggaggttttgggtcaccgggtgactttgccctgtctgtactcatcctggtctcacaacagcaacagcatgtgctgggggaaagaccagtgcccctactccggttgcaaggaggcgctcatccgcactgatggaatgagggtgacctcaagaaagtcagcaaaatatagacttcaggggactatcccgagaggtgatgtctccttgaccatcttaaaccccagtgaaagtgacagcggtgtgtactgctgccgcatagaagtgcctggctggttcaacgatgtaaagataaacgtgcgcctgaatctacagagagcctcaacaaccacgcacagaacagcaaccaccaccacacgcagaacaacaacaacaagccccaccaccacccgacaaatgacaacaaccccagctgcacttccaacaacagtcgtgaccacacccgatctcacaaccggaacaccactccagatgacaaccattgccgtcttcacaacagcaaacacgtgcctttcactaaccccaagcacccttccggaggaagccacaggtcttctgactcccgagccttctaaggaagggcccatcctcactgcagcatctgatacagcagttcctgagcagaacaaaacaacaaaaacaggacagatggatggaatacccatgtcaatgaagaatgaaatgcccatctcccaactactgatgatcatcgccccctccttgggatttgtgctcttcgcattgtttgtggcgtttctcctgagagggaaactcatggaaacctattgttcgcagaaacacacaaggctagactacattggagatagtaaaaatgtcctcaatgacgtgcagcatggaagggaagacgaagacggcctttttaccctctaacaacgcagtagcatgttagattgaggatgggggcatgacactccagtgtcaaaataagtcttagtagatttccttgtttcataaaaaagactcacttattccatggatgtcattgatccaggcttgctttagtttcatgaatgaagggtactttagagaccacaa